(19) United States
(12) Patent Application Publication
(10) Pub. No.: US 2023/0067676 A1
(43) Pub. Date: Mar. 2, 2023

(54) CO-CRYSTALS AND SALTS OF 8-CHLORO-N-(4-(TRIFLUOROMETHOXY)PHENYL)QUINOLIN-2-AMINE

(71) Applicants: ABIVAX, Paris (FR); CENTRE NATIONAL DE LA RECHERCHE SCIENTIFIQUE, Paris (FR); UNIVERSITE DE MONTPELLIER, Montpellier (FR); INSTITUT CURIE, Paris (FR)

(72) Inventors: Jérôme MENEGOTTO, Baziege (FR); Jérôme DENIS, Montferrier-sur-Lez (FR)

(73) Assignees: ABIVAX, Paris (FR); CENTRE NATIONAL DE LA RECHERCHE SCIENTIFIQUE, Paris (FR); UNIVERSITE DE MONTPELLIER, Montpellier (FR); INSTITUT CURIE, Paris (FR)

(21) Appl. No.: 17/793,133

(22) PCT Filed: Jan. 29, 2021
(86) PCT No.: PCT/EP2021/052165

§ 371 Date: Jul. 15, 2022

(30) Foreign Application Priority Data
Jan. 31, 2020 (EP) 20305090.1

(51) Int. Cl. C07D 215/38; A61K 9/48; G01N 33/50

(52) U.S. Cl. CPC C07D 215/38; A61K 9/4858; A61K 9/4866; A61K 9/4825; A61K 9/485; G01N 33/5082; C07B 2200/13

(57) ABSTRACT

Co-crystals and pharmaceutically acceptable salts and methods for the preparation of the co-crystals and the pharmaceutically acceptable salts of 8-chloro-N-(4-(trifluoromethoxy)phenyl)quinolin-2-amine, pharmaceutical compositions including them as well as their use as medicines and more particularly for use in the prevention and/or treatment of inflammatory diseases, diseases caused by viruses and/or cancer or dysplasia.

### FIELD OF THE INVENTION

The present invention relates to the pharmaceutical field and more particularly to novel co-crystals and novel pharmaceutically acceptable salts of 8-chloro-N-(4-(trifluoromethoxy)phenyl)quinolin-2-amine (also named ABX464), processes for their preparation, their use as medicines and in particular for preventing and/or treating of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia. The present invention also concerns pharmaceutical compositions comprising said co-crystals and/or pharmaceutically acceptable salts of 8-chloro-N-(4-(trifluoromethoxy)phenyl)quinolin-2-amine.

### BACKGROUND OF THE INVENTION

WO2010/143169 application describes the preparation and use of compounds, and in particular quinoline derivatives including certain pharmaceutically acceptable salts useful in the treatment of HIV infection. Said application in particular discloses 8-Chloro-N-(4-(trifluoromethoxy)phenyl)quinolin-2-amine also named (8-chloro-quinoline-2-yl)-(4-trifluoromethoxy-phenyl)-amine which is currently under clinical development. The inventors have stated that ABX464 is naturally highly crystalliferous and thus is spontaneously present under a specific unique stable and crystalline form named “crystalline form I”.

WO2017/158201 application deals with certain mineral acid or sulfonic acid salts of ABX464.

ABX464 has a poor solubility in aqueous solutions. The main drawback of said poor solubility is that the active ingredient cannot entirely reach their targets in the body if the drug remains undissolved in the gastrointestinal system.

There is therefore a need to provide new means for improving the solubility of ABX464.

### SUMMARY OF THE INVENTION

The inventors surprisingly found that implementation of new co-crystals and pharmaceutically acceptable salts of ABX464 provides new opportunities to improve the performance of a pharmaceutical product, for example in terms of solubility and controlling of dissolution rate. The inventors have now developed new pharmaceutically acceptable salts and new co-crystals of ABX464.

As mentioned above, there is always a need to improve the performance of a pharmaceutical product, for example in terms of solubility and more particularly when the free base is not water-soluble which is the case for ABX464. It has been observed that when the salts and co-crystals in accordance of the present disclosure are in suspension in water, due to the fact that the corresponding free base (that is to say 8-Chloro-N-(4-(trifluoromethoxy)phenyl)quinolin-2-amine itself) is very weak, they dissociate and the free base tends to precipitate. Consequently, to avoid the formation of a precipitate and such an instability during solubility measurements, these salts and co-crystals are classically formulated in the presence of at least one inhibitor of precipitation as more detailed herein after. As shown in the experimental part (example 10) the salts and co-crystals in accordance with the present disclosure have surprisingly a significant higher solubility compared to ABX464 Crystalline Form I (that is to say free base. In addition, the example 11 of the present disclosure demonstrates that the salts in accordance with the present disclosure have a surprisingly significant higher solubility compared to ABX464 Crystalline Form I (that is to say free base) in an intestinal compartment model.

Thus, the present invention is intended to provide co-crystals and pharmaceutically acceptable salts of 8-chloro-N-(4-(trifluoromethoxy)phenyl)quinolin-2-amine, as well as pharmaceutical compositions comprising said co-crystals and/or pharmaceutically acceptable salts of ABX464 including a solvate and/or a hydrate thereof. Said co-crystals, pharmaceutically acceptable salts of ABX464 and pharmaceutical compositions comprising them can be used as a medicament and more particularly for treating and/or preventing of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia.

The present invention thus provides co-crystals of 8-chloro-N-(4-(trifluoromethoxy)phenyl)quinolin-2-amine which are chosen among:
8-Chloro-N-(4-(trifluoromethoxy)phenyl)quinolin-2-amine: L-Proline having a powder X-ray diffractogram displaying peaks expressed as degree 2-Theta angle at 16.5; 20.6; 21.4; and 22.1 (each time ±0.2), and/or having a single endotherm with an onset temperature of 172.0° C. (±2° C.);
8-Chloro-N-(4-(trifluoromethoxy)phenyl)quinolin-2-amine: Gentisic acid having a powder X-ray diffractogram displaying peaks expressed as degree 2-Theta angle at 7.9; 14.0; 15.2; and 25.2 (each time ±0.2), and/or having a single endotherm with an onset temperature of 133.0° C. (±2° C.);
8-Chloro-N-(4-(trifluoromethoxy)phenyl)quinolin-2-amine: Malonic acid having a powder X-ray diffractogram displaying peaks expressed as degree 2-Theta angle at 9.5; 12.2; 15.8; 17.3; 19.7; 22.8; 24.8; and 25.6 (each time ±0.2), and/or having a single endotherm with an onset temperature of 109.0° C. (±2° C.); and
8-Chloro-N-(4-(trifluoromethoxy)phenyl)quinolin-2-amine: 4, 4′-Bipyridine having a powder X-ray diffractogram displaying peaks expressed as degree 2-Theta angle at 12.0; 19.2; 21.2; and 24.3 (each time ±0.2), and/or having a single endotherm with an onset temperature of 127.0° C. (±2° C.).

The present invention thus also provides a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate and the like, phosphate and the like, camphorsulfonate, 2-hydroxy-ethanesulfonate, estolate, napsylate, esylate, napadisylate, dodecylsulfate and the like, perchloric acid, boric acid, glycerophosphoric acid, nitric acid, persulfuric acid and the like.

Herein provided are also:
a method for preparing said co-crystals of 8-chloro-N-(4-(trifluoromethoxy)phenyl)quinolin-2-amine as defined in the present invention;
a method for preparing said pharmaceutically acceptable salts of 8-chloro-N-(4-(trifluoromethoxy)phenyl)quinolin-2-amine including a solvate and/or a hydrate thereof as defined in the present invention;
pharmaceutical compositions comprising said co-crystals as defined in the present invention and/or said pharmaceutically acceptable salts of ABX464 including a solvate and/or a hydrate thereof as defined in the present invention and at least one pharmaceutically acceptable excipient;
said pharmaceutically acceptable salt of ABX464 including a solvate and/or a hydrate thereof as defined in the present invention, said co-crystals as defined in the present invention or the pharmaceutical compositions as defined in the present invention for use as a medicine; and
said pharmaceutically acceptable salt of ABX464 including a solvate and/or a hydrate thereof as defined in the present invention, said co-crystals as defined in the present invention or the pharmaceutical compositions as defined in the present invention for use in the prevention or treatment of cancer, AIDS, an HIV infection, and/or inflammatory diseases.

A co-crystal is a crystal complex composed of at least two neutral molecules bound together in a crystal lattice by non-covalent interactions. The main difference between solvates and co-crystals is related to the physical state of the pure components: if one of the constituents is liquid at ambient temperature, the molecular complex is then a solvate; if all the components are solid at ambient temperature, the complex is then designated by the term “co-crystal”. The major difference between a solvate and a co-crystal is the much greater stability of the co-crystals compared to the solvate. A co-crystal is characterised by the method by which it is obtained and by an ordered three-dimensional structure which is demonstrated, for example, by X-ray diffraction diagrams. It is not possible to know a priori whether two given constituents will be able to form a co-crystal having a particular three-dimensional structure or will simply give rise to a juxtaposition of the two powders.

As used herein, the term “pharmaceutically acceptable” refers to those compounds, materials, excipients, carrier, adjuvant, vehicle, compositions or dosage forms which are, within the scope of sound medical judgment, suitable for contact with the tissues of human beings and animals without excessive toxicity, irritation, allergic response or other problem complications commensurate with a reasonable benefit/risk ratio.

In the context of the invention, the term “treating” or “treatment”, as used herein, means reversing, alleviating, inhibiting the progress of, or preventing of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia.

The term “preventing”, as used herein, means reducing the risk of onset or slowing the occurrence of a given phenomenon, namely in the present invention, of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia. As used herein, «preventing» also encompasses «reducing the likelihood of occurrence» or «reducing the likelihood of reoccurrence».

A “subject” (including patient) includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like).

As used herein, the term “ambient temperature” or “room temperature” refers to a temperature ranging from 15° C. to 30° C., more particularly from 18° C. to 25° C.

### BRIEF DESCRIPTION OF THE FIGURES

FIG. 1 is a X-ray powder diagram of a co-crystal of 8-Chloro-N-(4-(trifluoromethoxy)phenyl)quinolin-2-amine: L-Proline (see example 1).

FIG. 2 is a X-ray powder diagram of a co-crystal of 8-Chloro-N-(4-(trifluoromethoxy)phenyl)quinolin-2-amine: Gentisic acid (see example 2).

FIG. 3 is a X-ray powder diagram of a co-crystal of 8-Chloro-N-(4-(trifluoromethoxy)phenyl)quinolin-2-amine: Malonic acid (see example 3).

FIG. 4 is a X-ray powder diagram of a co-crystal of 8-Chloro-N-(4-(trifluoromethoxy)phenyl)quinolin-2-amine: 4,4′-Bipyridine (see example 4).

FIG. 5 is a X-ray powder diagram of anhydrous crystalline ABX464 hemi-napadisylate salt (see example 5).

FIG. 6 is a X-ray powder diagram of anhydrous crystalline ABX464 esylate salt (see example 6).

FIG. 7 is a X-ray powder diagram of crystalline hemi-THF (tetrahydrofuran) solvate of ABX464 hemi-napadisylate (see example 7).

FIG. 8 represents three X-ray powder diagrams, the first recorded for ABX464 crystalline Form I (see top line), the second recorded for solid residues collected at time point G 30 min in Gastric compartment (see middle line), and the third recorded for solid residues collected at time point I 120 min in Intestinal compartment (see bottom line) of Fasted Dissolution/Precipitation tests done for ABX464 crystalline Form I. (see example 11).

FIG. 9 represents four X-ray powder diagrams, the first recorded for ABX464 crystalline Form I (see top line), the second recorded for anhydrous ABX464 hemi-Napadisylate salt (see second line numbered from the top), the third recorded for solid residues collected at time point G 30 min in Gastric compartment (see third line numbered from the top), and the fourth recorded for solid residues collected at time point I 120 min in Intestinal compartment (see bottom line, i.e. the fourth line numbered from the top) of Fasted Dissolution/Precipitation tests done for anhydrous ABX464 hemi-Napadisylate salt. (see example 11).

### DETAILED DESCRIPTION OF THE INVENTION

As explained above, the inventors have demonstrated that the salts and co-crystals in accordance with the present disclosure demonstrate improved solubility in comparison to the solubility of ABX464 form I crystalline form.

As it will come out through the example 10 herein after, said improved solubility has been studied in two typical media FaSSIF (namely Fasted State Simulated Intestinal Fluid) and FeSSIF (namely Fed State Simulated Intestinal Fluid).

As it will be apparent from said example, an inhibitor or precipitation is also present in the media, and namely (polyvinylpyrrolidone-vinyl acetate) PVP-VA. The use of such an inhibitor of precipitation is classically used in the well-known “spring and parachute” approach. Indeed, to enhance the intestinal absorption of poorly water-soluble drugs, the concept of supersaturation in the gastrointestinal tract can be used as a strategy. To exploit supersaturation, two essential steps need to be considered: generation and maintenance of the metastable supersaturated state what is also named the Spring and Parachute Approach (see Guzmán et al: A “spring and parachute” approach to designing solid celecoxib formulations having enhanced oral absorption. AAPS J 6, 2004, Abstract T2189). Once supersaturation (called Spring) has been induced, drug molecules have the tendency to precipitate, through processes which may be kinetically or thermodynamically controlled. In order to benefit from the supersaturated state, the increased concentrations have to be maintained for a time period sufficient for absorption. This may require a temporary inhibition of precipitation through the use of pharmaceutical excipients or by other components that interfere with nucleation and/or crystal growth, that is, the “parachutes” or precipitation inhibitors.

Among the inhibitors of precipitation may be cited polymers such as PVP-VA (polyvinylpyrrolidone-vinyl acetate), HPMC (hydroxypropyl methyl cellulose), HPMC-AS, HPMC-P, Surfactants such as TPGS (d-alpha-tocopheryl polyethylene glycol 1000 succinate) or Pluronic® F127 (also named P407: poloxamer 407), hydroxypropyl cellulose (HPC), PVP (polyvinyl pyrrolidone) such as PVP-K15, PVP-K30 or PVP-K90, Carbopol® 974P (highly carboxylated polymer composed of lightly cross-linked polyacrylic acid), polyvinyl alcohol (PVA) and mixtures thereof, or also may be cited sodium dodecyl sulfate (SDS).

As illustrated in example 10, there has been observed a significant higher solubility of all solid forms in FeSSIF pH5.0+1% PVPVA medium, mimicking the fed intestinal medium. In other words, the presence of an inhibitor of precipitation in said media prevents precipitation of ABX464 free base but also opens the route for implementing it in new galenic forms.

Example 11, testing the solubility behavior of a salt of ABX464 in a two Step Dissolution-Precipitation Fasted Human in vitro model (see for instance, Dressman et al., “Estimating drug solubility in the gastrointestinal tract”, Advanced Drug Delivery Reviews, 2007, volume 59, issue 7, pp. 591-602) further validates that the salts of ABX464 according to the present disclosure demonstrate an improved solubility in comparison to the ABX464 Crystalline Form I. Indeed, a significant higher solubility of ABX464 hemi-Napadisylate salt (˜430 μg/ml) in the intestinal compartment compared to the solubility of ABX464 Crystalline Form I (˜80 μg/ml) in the intestinal compartment of this model was clearly observed.

Therefore, the inventors have discovered new means for increasing the solubility of ABX464, also for allowing the preparation of a greater variety of galenic formulations ensuring that ABX464 reaches its biological target with the best bioavailability, as detailed herein after in the paragraph “PHARMACEUTICAL COMPOSITIONS”.

Salts and co-crystals of ABX464 subject-matter of the present invention are described in more details herein after.

Salts of ABX464

As mentioned above, an object of the present invention is a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate and the like, phosphate and the like, camphorsulfonate, 2-hydroxy-ethanesulfonate, estolate, napsylate, esylate, napadisylate, dodecylsulfate and the like, perchloric acid, boric acid, glycerophosphoric acid, nitric acid, persulfuric acid and the like, particularly is selected from esylate and napadisylate, more particularly is selected from anhydrous crystalline ABX464 hemi-napadisylate salt, anhydrous crystalline ABX464 esylate salt, and crystalline hemi-THF solvate of ABX464 hemi-napadisylate.

Thus, according to one embodiment, said pharmaceutically acceptable salt is anhydrous and is selected from anhydrous crystalline ABX464 hemi-napadisylate salt and anhydrous crystalline ABX464 esylate salt.

Thus, according to another embodiment, said pharmaceutically acceptable salt is under the form of a solvate or a hydrate, more particularly a solvate and still more particularly is crystalline hemi-THF (tetrahydrofuran) solvate of ABX464 hemi-napadisylate.

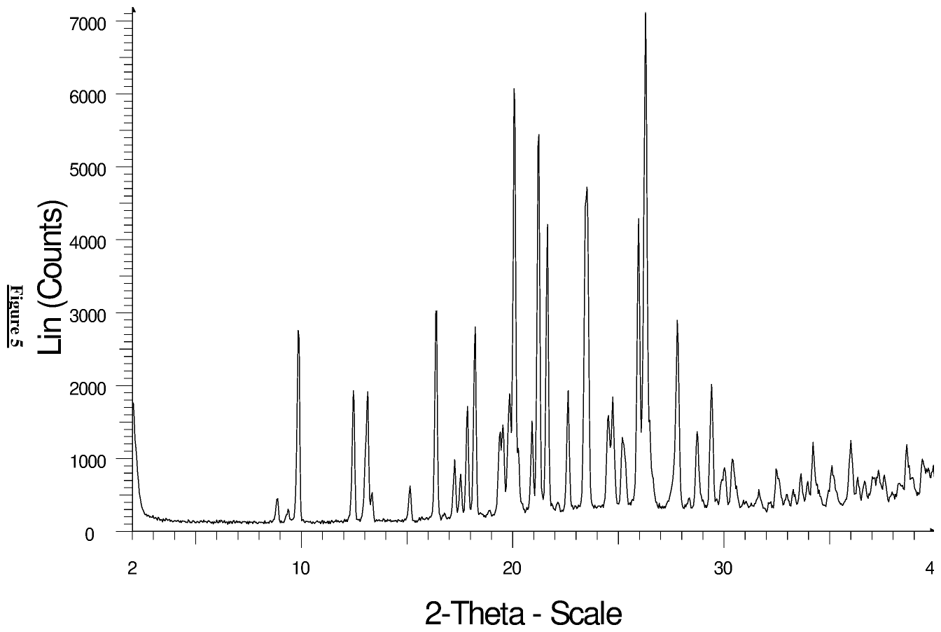

Hence, in a particular embodiment, a pharmaceutically acceptable salt of 8-chloro-N-(4-(trifluoromethoxy)phenyl)quinolin-2-amine as defined in the present invention is selected from anhydrous crystalline ABX464 hemi-napadisylate salt, anhydrous crystalline ABX464 esylate salt, and crystalline hemi-THF solvate of ABX464 hemi-napadisylate, as illustrated respectively in examples 5, 6 and 7 (and respectively FIGS. 5, 6 and 7) of the present text.

In a particularly preferred embodiment, a pharmaceutically acceptable salt of 8-chloro-N-(4-(trifluoromethoxy)phenyl)quinolin-2-amine as defined in the present invention is anhydrous crystalline ABX464 hemi-napadisylate salt.

The pharmaceutically acceptable salts of 8-chloro-N-(4-(trifluoromethoxy)phenyl)quinolin-2-amine may be for example characterized by X-Ray Powder Diffraction (XRPD) and by Differential Scanning calorimetry (DSC).

More particularly, anhydrous crystalline ABX464 hemi-napadisylate salt of example 5 has a powder X-ray diffractogram displaying peaks expressed as degree 2-Theta angle at 9.8; 16.4; 18.2; 20.1; 21.2; 21.6; 23.5 and 26.3 (each time ±0.2), and optionally further shows the following additional peaks expressed as degree 2-Theta angle: 12.4; 13.1; 17.8; 20.9; 22.6; 24.5; 24.7; 25.2; and 25.9 (each time ±0.2); and even optionally further the following additional peaks expressed as degree 2-Theta angle: 8.8; 13.3; 15.1; 17.2; 17.5; 19.4; 19.5; and 19.8 (each time ±0.2), as illustrated in FIG. 5 (powder X-ray diffractogram) and/or has a single endotherm with an onset temperature of 269.0° C. (±2° C.).

A characteristic X-ray powder diffractogram of an anhydrous crystalline ABX464 hemi-napadisylate salt can be given in FIG. 5 and its characteristic signals are summarized in the following table 1:

TABLE 1

| Angle (2-Theta) | Relative intensity |
|---|---|
| (each time ± 0.2) | (%) |
| 8.8 | 6 |
| 9.8 | 38 |
| 12.4 | 27 |
| 13.1 | 27 |
| 13.3 | 7 |
| 15.1 | 8 |
| 16.4 | 42 |
| 17.2 | 13 |
| 17.5 | 11 |
| 17.8 | 24 |
| 18.2 | 39 |
| 19.4 | 20 |
| 19.5 | 20 |
| 19.8 | 26 |
| 20.1 | 85 |
| 20.9 | 21 |
| 21.2 | 76 |
| 21.6 | 59 |
| 22.6 | 27 |
| 23.5 | 66 |
| 24.5 | 22 |
| 24.7 | 25 |
| 25.2 | 18 |
| 25.9 | 60 |
| 26.3 | 100 |

According to one embodiment, the anhydrous crystalline ABX464 hemi-napadisylate salt of the present invention presents a powder X-ray diffractogram displaying at least one peak expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least two peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least three peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least four peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least five peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least six peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least seven peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least eight peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least nine peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least ten peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least eleven peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least twelve peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least thirteen peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least fourteen peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention presents a powder X-ray diffractogram displaying at least fifteen peaks expressed as degree 2-Theta angle selected from 9.8; 16.4; 18.2; 20.1; 21.2; 21.6; 23.5; 26.3; 12.4; 13.1; 17.8; 20.9; 22.6; 24.5; 24.7; 25.2; 25.9; 8.8; 13.3; 15.1; 17.2; 17.5; 19.4; 19.5; and 19.8 (each time ±0.2).

According to another embodiment, the anhydrous crystalline ABX464 hemi-napadisylate salt of the present invention has an XRPD substantially similar to that depicted in FIG. 5.

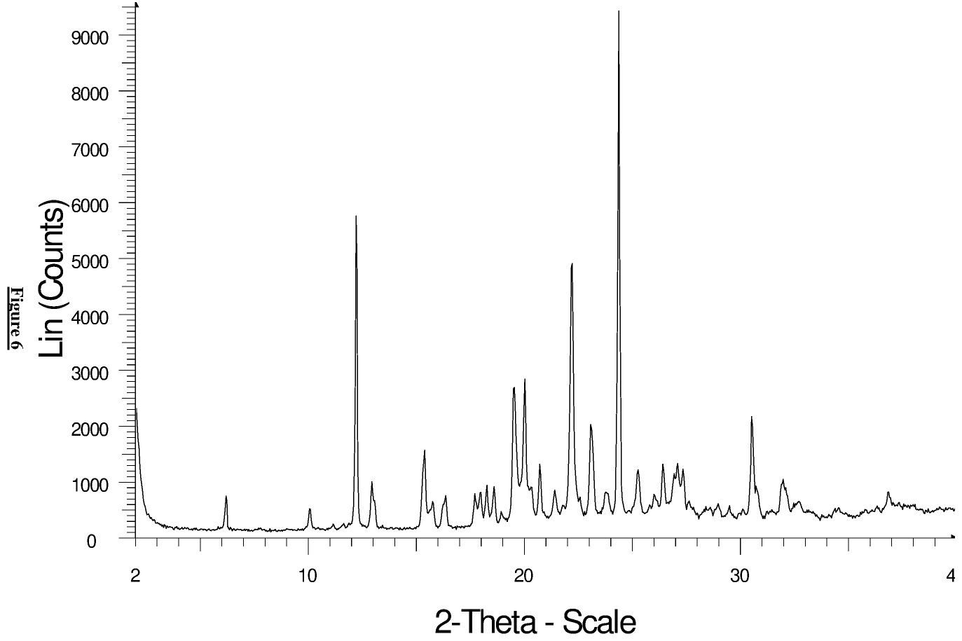

More particularly, anhydrous crystalline ABX464 esylate salt of example 6 has a powder X-ray diffractogram displaying peaks expressed as degree 2-Theta angle at 12.2; and 22.2 (each time ±0.2), and optionally further shows the following additional peaks expressed as degree 2-Theta angle: 6.2; 12.9; 13.1; 15.3; 16.3; 18.2; 18.6; 19.5; 20.0; and 20.7 (each time ±0.2); and even optionally further the following additional peaks expressed as degree 2-Theta angle: 10.1; 15.8; 17.7; 17.9; 20.3; and 21.4 (each time ±0.2), as illustrated in FIG. 6 (powder X-ray diffractogram) and/or has a single endotherm with an onset temperature of 108.0° C. (±2° C.).

A characteristic X-ray powder diffractogram of an anhydrous crystalline ABX464 esylate salt can be given in FIG. 6 and its characteristic signals are summarized in the following table 2:

TABLE 2

| Angle (2-Theta) | Relative intensity |
|---|---|
| (each time ± 0.2) | (%) |
| 6.2 | 13 |
| 10.1 | 9 |
| 12.2 | 100 |
| 12.9 | 17 |
| 13.1 | 11 |
| 15.3 | 27 |
| 15.8 | 11 |
| 16.3 | 13 |
| 17.7 | 14 |
| 17.9 | 14 |
| 18.2 | 16 |
| 18.6 | 15 |
| 19.5 | 47 |
| 20.0 | 49 |
| 20.3 | 15 |
| 20.7 | 23 |
| 21.4 | 15 |
| 22.2 | 85 |

According to one embodiment, the anhydrous crystalline ABX464 esylate salt of the present invention presents a powder X-ray diffractogram displaying at least one peak expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least two peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least three peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least four peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least five peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least six peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least seven peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least eight peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least nine peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least ten peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least eleven peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least twelve peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least thirteen peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least fourteen peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention presents a powder X-ray diffractogram displaying at least fifteen peaks expressed as degree 2-Theta angle selected from 12.2; 22.2; 6.2; 12.9; 13.1; 15.3; 16.3; 18.2; 18.6; 19.5; 20.0; 20.7; 10.1; 15.8; 17.7; 17.9; 20.3; and 21.4 (each time ±0.2).

According to another embodiment, the anhydrous crystalline ABX464 esylate salt of the present invention has an XRPD substantially similar to that depicted in FIG. 6.

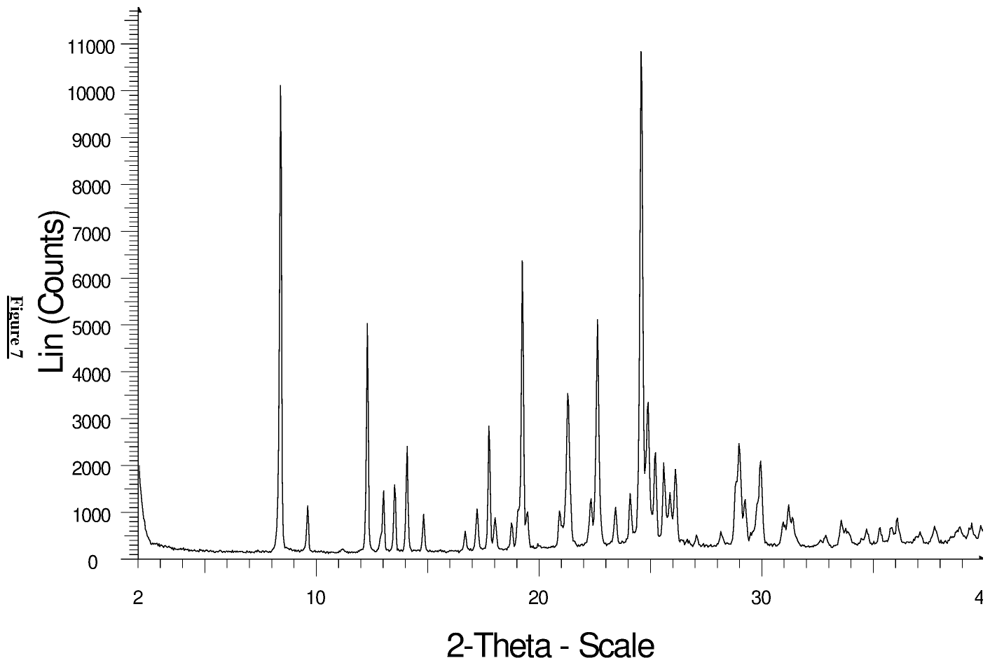

More particularly, crystalline hemi-THF solvate of ABX464 hemi-napadisylate salt of example 7 has a powder X-ray diffractogram displaying peaks expressed as degree 2-Theta angle at 8.4; 12.3; 14.0; 19.2; 21.3; 22.6 and 24.6 (each time ±0.2), and optionally further shows the following additional peaks expressed as degree 2-Theta angle: 9.6; 13.0; 13.5; 14.8; 17.2; 17.8; 23.4; 24.1; 24.9 and 25.2 (each time ±0.2); and even optionally further the following additional peaks expressed as degree 2-Theta angle: 16.7; 18.1; 18.8; 19.5; 20.9 and 22.3 (each time ±0.2), as illustrated in FIG. 7 (powder X-ray diffractogram) and/or has a single endotherm with an onset temperature of 172.0° C. (±2° C.).

A characteristic X-ray powder diffractogram of a crystalline hemi-THF solvate of ABX464 hemi-napadisylate salt can be given in FIG. 7 and its characteristic signals are summarized in the following table 3:

TABLE 3

| Angle (2-Theta) | Relative intensity |
|---|---|
| (each time ±0.2) | (%) |
| 8.4 | 93 |
| 9.6 | 10 |
| 12.3 | 46 |
| 13.0 | 13 |
| 13.5 | 14 |
| 14.0 | 22 |
| 14.8 | 9 |
| 16.7 | 5 |
| 17.2 | 10 |
| 17.8 | 26 |
| 18.1 | 8 |
| 18.8 | 7 |
| 19.2 | 59 |
| 19.5 | 9 |
| 20.9 | 9 |
| 21.3 | 32 |
| 22.3 | 12 |
| 22.6 | 47 |
| 23.4 | 10 |
| 24.1 | 13 |
| 24.6 | 100 |
| 24.9 | 31 |
| 25.2 | 21 |

According to one embodiment, the crystalline hemi-THF solvate of ABX464 hemi-napadisylate salt of the present invention presents a powder X-ray diffractogram displaying at least one peak expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least two peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least three peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least four peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least five peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least six peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least seven peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least eight peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least nine peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least ten peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least eleven peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least twelve peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least thirteen peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least fourteen peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention presents a powder X-ray diffractogram displaying at least fifteen peaks expressed as degree 2-Theta angle selected from 8.4; 12.3; 14.0; 19.2; 21.3; 22.6; 24.6; 9.6; 13.0; 13.5; 14.8; 17.2; 17.8; 23.4; 24.1; 24.9; 25.2; 16.7; 18.1; 18.8; 19.5; 20.9 and 22.3 (each time ±0.2).

According to another embodiment, the crystalline hemi-THF solvate of ABX464 hemi-napadisylate salt of the present invention has an XRPD substantially similar to that depicted in FIG. 7.

Method for Preparing a Pharmaceutically Acceptable Salt of ABX464

Herein is further provided a method for preparing a pharmaceutically acceptable salt of ABX464 (including solvate or hydrate thereof) which comprises the following steps:

a) dissolving ABX464 in a solvent or in a mixture of solvents;

b) adding to the thus obtained mixture of step a) a counter ion under the form of an acid which may be itself already dissolved in a solvent or in a mixture of solvents so as to obtain a ABX464: counter ion molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent or a mixture of solvents,

e) applying a temperature program;

f) optionally filtrating; and

g) then optionally drying at a temperature comprised between room temperature and 60° C. in order to obtain the desired salt of ABX464.

According to one embodiment, the solvent(s) used in step a), step b) and step d) is(are) any solvent conventionally used in crystallization step, particularly is(are) organic solvents, more particularly is(are) selected from a C₁-C₆ aliphatic alcohol, methyl ethyl ketone (also named butanone or MEK), cyclohexane, alkane such as heptane, methylene chloride, chloroform, formic acid, DMSO, 1-methyl-2-pyrrolidone, acetone, acetonitrile, tetrahydrofuran (THF), diethyl ether, dioxane, toluene, ethyl acetate, optionally in admixture with water, and mixtures thereof, still more particularly selected from a C₁-C₆ aliphatic alcohol, a mixture of H₂O/C₁-C₆ aliphatic alcohol, and mixtures thereof, even more particularly selected from methanol, ethanol, isopropanol, H₂O/methanol, H₂O/ethanol, and mixtures thereof.

The skilled person would know how to determine the more appropriate solvent(s) in each step a), b) and d) so as to obtain the desired pharmaceutically acceptable salt.

According to one embodiment, the solvent(s) used in step a), step b) and/or step d) is(are) the same.

According to another embodiment, the solvent(s) used in step a), step b) and/or step d) is(are) different.

Advantageously, the counter ion under the form of an acid of step b) is ethane sulfonic acid or naphthalene1,5-disulfonic acid.

Thus, in some embodiments, the solvent of step a), step b) and/or step d) is any solvent conventionally used in the crystallization step, particularly is organic solvents, more particularly is selected from a C₁-C₆ aliphatic alcohol, methyl ethyl ketone (also named butanone or MEK), cyclohexane, alkane such as heptane, methylene chloride, chloroform, formic acid, DMSO, 1-methyl-2-pyrrolidone, acetone, acetonitrile, tetrahydrofuran (THF), diethyl ether, dioxane, toluene, ethyl acetate, optionally in admixture with water, and mixtures thereof, still more particularly selected from a C₁-C₆ aliphatic alcohol, a mixture of H₂O/C₁-C₆ aliphatic alcohol, and mixtures thereof, even more particularly selected from methanol, ethanol, isopropanol, H₂O/methanol, H₂O/ethanol, and mixtures thereof; and/or the counter ion under the form of an acid of step b) is ethane sulfonic acid or naphthalene1,5-disulfonic acid.

According to one embodiment, the evaporating step c) is carried out under an inert gas such as N₂.

According to one embodiment the step e) relative to the temperature program includes a heating phase at reflux to a temperature comprised between room temperature and the boiling point of the solvent(s), particularly between room temperature and 60° C.

According to one embodiment the step e) relative to the temperature program includes a cooling phase at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min.

According to one embodiment, the step e) relative to the temperature program includes i) a heating at reflux to a temperature comprised between room temperature and the boiling point of the solvent(s), particularly between room temperature and 60° C. and/or ii) a cooling at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min.

According to a particular embodiment, the step e) relative to the temperature program includes i) a heating at reflux to a temperature comprised between room temperature and 60° C. and ii) a cooling at reflux to a temperature comprised between 5° C. and 40° C., at a rate comprised between 30° C./min and 0.05° C./min, particularly between 10° C./min and 0.05° C./min, more particularly between 5° C./min and 0.05° C./min.

According to one embodiment, the step f) regarding the filtration is carried out using conventional glass fibers, conventional cellulosic filter papers, PTFE (polytetrafluoroethylene), or PVDF (polyvinylidene fluoride), particularly cellulosic filter paper with 0.45 μm or 0.2 μm filtration mesh.

According to one embodiment, the step g) regarding the drying is carried out under vacuum at a temperature comprised between 30° C. and 60° C., particularly at 40° C.

According to one embodiment, the step g) regarding the drying is carried out under ambient atmosphere at a temperature comprised between 30° C. and 60° C., particularly at 40° C.

It is understood that for the preparation of a pharmaceutically acceptable salt of ABX464 as defined in the present invention, 8-chloro-N-(4-(trifluoromethoxy)phenyl)quinolin-2-amine can be previously obtained either from a process as the one described in WO2010/143169 or from any other appropriate process.

According to some embodiments, the method for preparing a pharmaceutically acceptable salt of ABX464 (including solvate or hydrate thereof) comprises the following steps:

a) dissolving ABX464 in a solvent or in a mixture of solvents;

b) adding to the thus obtained mixture of step a) a counter ion under the form of an acid which may be itself already dissolved in a solvent or in a mixture of solvents so as to obtain a ABX464: counter ion molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent or a mixture of solvents,

e) applying a temperature program which includes i) a heating at reflux to a temperature comprised between room temperature and the boiling point of the solvent(s), particularly between room temperature and 60° C. and/or ii) a cooling at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min.;

f) optionally filtrating by using conventional glass fibers, conventional cellulosic filter papers, PTFE (polytetrafluoroethylene), or PVDF (polyvinylidene fluoride), particularly cellulosic filter paper with 0.45 μm or 0.2 μm filtration mesh; and

g) then optionally drying under vacuum at a temperature comprised between 30° C. and 60° C., particularly at 40° C. or under ambient atmosphere at a temperature comprised between 30° C. and 60° C., particularly at 40° C. in order to obtain the desired salt of ABX464.

According to some embodiments, the method for preparing a pharmaceutically acceptable salt of ABX 464 comprises the following steps:

a) dissolving ABX464 in methanol;

b) adding to the thus obtained mixture of step a) a counter ion which is ethane sulfonic acid or naphthalene sulfonic acid which is itself already dissolved in water\ethanol or ethanol so as to obtain a ABX464: counter ion molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent selected from ethyl acetate, THF, acetone, and mixtures thereof,

e) applying a temperature program which includes (i) a heating phase at reflux to a temperature comprised between room temperature and the boiling point of the solvent( ) particularly between room temperature and 60° C. and/or (ii) a cooling phase at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min;

f) optionally filtrating; and

g) then optionally drying at a temperature comprised between room temperature and 60° C. in order to obtain the desired salt of ABX464.

According to some embodiments, the method for preparing a pharmaceutically acceptable salt of ABX 464 comprises the following steps:

a) dissolving ABX464 in methanol;

b) adding to the thus obtained mixture of step a) a counter ion which is ethane sulfonic acid or naphthalene sulfonic acid which is itself already dissolved in water\ethanol or ethanol so as to obtain a ABX464: counter ion molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent selected from ethyl acetate, THF, acetone, and mixtures thereof,

e) applying a temperature program which includes (i) a heating phase at reflux to a temperature of 60° C. and/or (ii) a cooling phase at reflux to a temperature of 5° C. and 40° C., more particularly between room temperature and 40° C. at a rate of 1° C./min;

f) optionally filtrating; and

g) then optionally drying at 40° C. in order to obtain the desired salt of ABX464.

According to one particular embodiment, ABX464 is dissolved in a C₁-C₆ aliphatic alcohol, in particular methanol, then to this mixture is added naphthalene1,5-disulfonic acid which is itself already dissolved in a C₁-C₆ aliphatic alcohol, in particular ethanol so as to obtain a ABX464: counter ion molar ratio comprised between 3:1 and 1:2, particularly between 5:2 and 1:2; more particularly between 2:1 and 1:2, and still more particularly is of 2:1 or 1:1. Then evaporation under N₂ at room temperature (25° C.), then addition of acetone, then applying a temperature program before filtration on 0.2 μm, and drying on vacuum at 40° C. are carried out to obtain anhydrous crystalline ABX464 hemi-napadisylate salt.

According to another particular embodiment, ABX464 is dissolved in a C₁-C₆ aliphatic alcohol, in particular methanol, then to this mixture is added ethane sulfonic acid which is itself already dissolved in a mixture H₂O/C₁-C₆ aliphatic alcohol, in particular water/ethanol so as to obtain a ABX464: counter ion molar ratio between 3:1 and 1:2, particularly between 5:2 and 1:2; more particularly between 2:1 and 1:2, and still more particularly is of 1:1. Then evaporation under N₂ at room temperature (25° C.), then addition of ethyl acetate, then applying a temperature program before evaporation carried out under N₂ at room temperature (25° C.) are carried out to obtain anhydrous crystalline ABX464 esylate salt.

According to another embodiment, ABX464 is dissolved in a C₁-C₆ aliphatic alcohol, in particular methanol, then to this mixture is added naphthalene1,5-disulfonic acid which is itself already dissolved in a C₁-C₆ aliphatic alcohol, in particular ethanol so as to obtain a ABX464: counter ion molar ratio between 3:1 and 1:2, particularly between 5:2 and 1:2; more particularly between 2:1 and 1:2, and still more particularly is of 2:1. Then evaporation under N₂ at 40° C., then addition of THF, then a temperature program is applied before filtration on 0.2 μm, and drying on vacuum at 40° C. are carried out to obtain crystalline hemi-THF solvate of ABX464 hemi-napadisylate.

Co-Crystals of ABX464

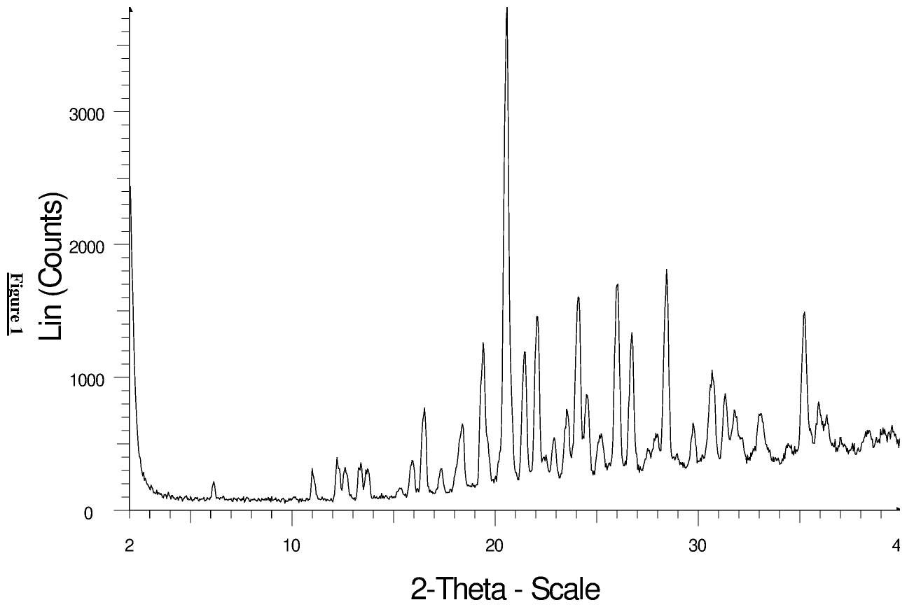

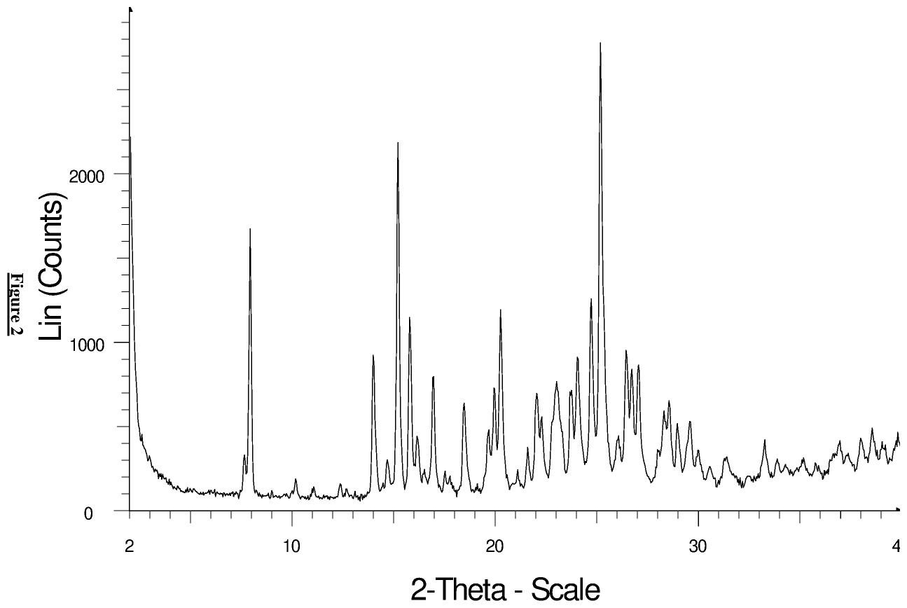

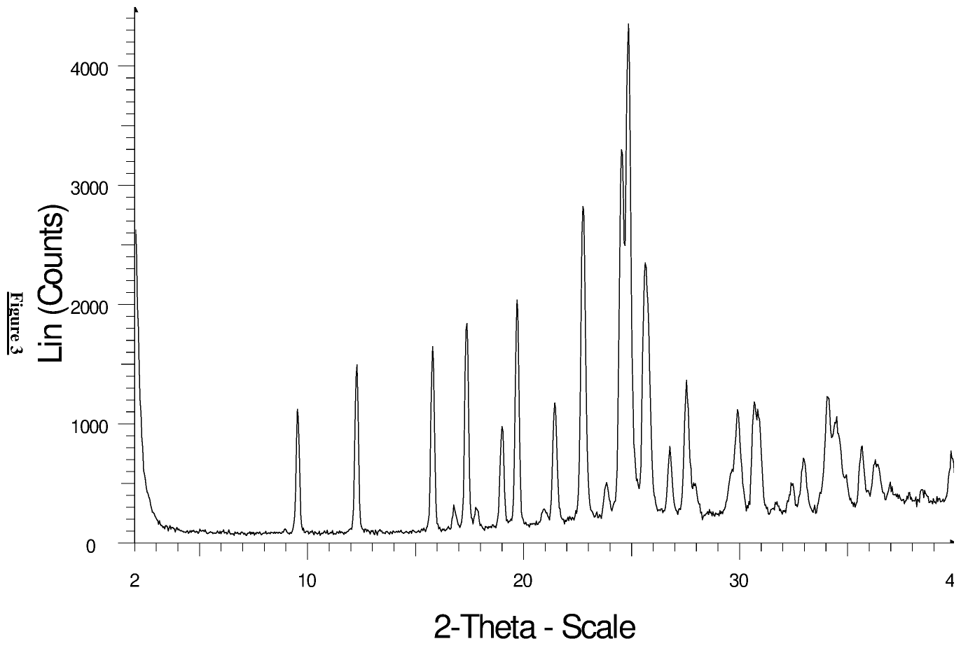

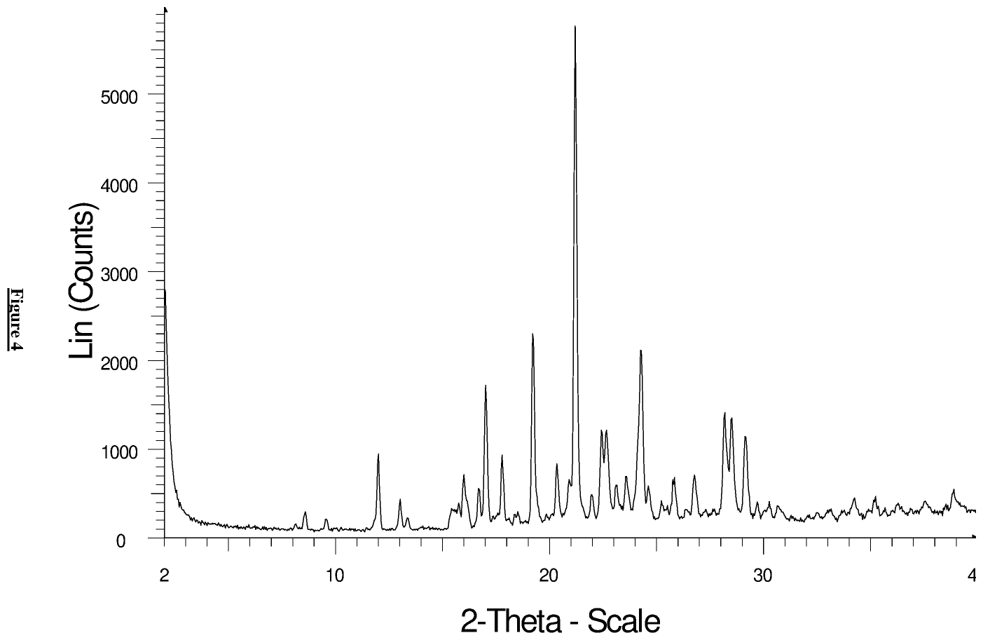

As mentioned above, in another aspect, the present invention relates to co-crystals of 8-chloro-N-(4-(trifluoromethoxy)phenyl)quinolin-2-amine which are chosen among:
8-Chloro-N-(4-(trifluoromethoxy)phenyl)quinolin-2-amine: L-Proline having a powder X-ray diffractogram displaying peaks expressed as degree 2-Theta angle at 16.5; 20.6; 21.4; and 22.1 (each time ±0.2), and which may optionally further show the following additional peaks expressed as degree 2-Theta angle: 11.0; 15.9; 18.3; and 19.4 (each time ±0.2); and even optionally further the following additional peaks expressed as degree 2-Theta angle 6.1; 12.2; 12.6; 13.3; 13.7; 15.4; 17.3 and 22.4 (each time ±0.2), optionally further characterized by a powder X-ray diffractogram as illustrated in FIG. 1 and/or having a single endotherm with an onset temperature of 172.0° C. (±2° C.);
8-Chloro-N-(4-(trifluoromethoxy)phenyl)quinolin-2-amine: Gentisic acid having a powder X-ray diffractogram displaying peaks expressed as degree 2-Theta angle at 7.9; 14.0; 15.2; and 25.2 (each time ±0.2), and which may optionally further show the following additional peaks expressed as degree 2-Theta angle: 15.8; 16.9; 18.5; 19.9; 20.3; 23.0 and 24.7 (each time ±0.2); and even optionally further the following additional peaks expressed as degree 2-Theta angle: 7.6; 14.7; 16.1; 19.7; 21.6; 22.0; 22.3; 23.7; and 24.0 (each time ±0.2), optionally further characterized by a powder X-ray diffractogram as illustrated in FIG. 2 and/or having a single endotherm with an onset temperature of 133.0° C. (±2° C.);
8-Chloro-N-(4-(trifluoromethoxy)phenyl)quinolin-2-amine: Malonic acid having a powder X-ray diffractogram displaying peaks expressed as degree 2-Theta angle at 9.5; 12.2; 15.8; 17.3; 19.7; 22.8; 24.8; and 25.6 (each time ±0.2), and which may optionally further show the following additional peaks expressed as degree 2-Theta angle: 19.0; 21.4; 24.6; 26.8; 27.6; and 29.9 (each time ±0.2); and even optionally further the following additional peaks expressed as degree 2-Theta angle 16.8; 17.8; 20.9; 23.8; 28.0; and 29.6 (each time ±0.2), optionally further characterized by a powder X-ray diffractogram as illustrated in FIG. 3 and/or having a single endotherm with an onset temperature of 109.0° C. (±2° C.); and
8-Chloro-N-(4-(trifluoromethoxy)phenyl)quinolin-2-amine: 4, 4′-Bipyridine having a powder X-ray diffractogram displaying peaks expressed as degree 2-Theta angle at 12.0; 19.2; 21.2; and 24.3 (each time ±0.2), and which may optionally further show the following additional peaks expressed as degree 2-Theta angle: 16.0; 17.0; 17.8; 20.3; 22.5; and 22.7 (each time ±0.2); and even optionally further the following additional peaks expressed as degree 2-Theta angle: 8.5; 13.0; 15.7; 16.7; 20.9; 22.0; 23.1; 23.6 and 24.7 (each time ±0.2), optionally further characterized by a powder X-ray diffractogram as illustrated in FIG. 4 and/or having a single endotherm with an onset temperature of 127.0° C. (±2° C.).

In a particularly preferred embodiment, a co-crystal of 8-chloro-N-(4-(trifluoromethoxy)phenyl)quinolin-2-amine is 8-Chloro-N-(4-(trifluoromethoxy)phenyl)quinolin-2-amine: L-Proline having a powder X-ray diffractogram displaying peaks expressed as degree 2-Theta angle at 16.5; 20.6; 21.4; and 22.1 (each time ±0.2), and which may optionally further show the following additional peaks expressed as degree 2-Theta angle: 11.0; 15.9; 18.3; and 19.4 (each time ±0.2); and even optionally further the following additional peaks expressed as degree 2-Theta angle 6.1; 12.2; 12.6; 13.3; 13.7; 15.4; 17.3 and 22.4 (each time ±0.2), optionally further characterized by a powder X-ray diffractogram as illustrated in FIG. 1 and/or having a single endotherm with an onset temperature of 172.0° C. (±2° C.).

Said four co-crystals are respectively illustrated in examples 1 (and FIG. 1), 2 (and FIG. 2), 3 (and FIG. 3) and 4 (and FIG. 4) of the present text.

A characteristic X-ray powder diffractogram of a co-crystal of 8-chloro-N-(4-(trifluoromethoxy)phenyl)quinolin-2-amine: L-Proline can be given in FIG. 1 and its characteristic signals are summarized in the following table 4:

TABLE 4

| Angle (2-Theta) | Relative intensity |
|---|---|
| (each time ±0.2) | (%) |
| 6.1 | 5 |
| 11.0 | 8 |
| 12.2 | 10 |
| 12.6 | 8 |
| 13.3 | 9 |
| 13.7 | 8 |
| 15.4 | 4 |
| 15.9 | 9 |
| 16.5 | 20 |
| 17.3 | 8 |
| 18.3 | 17 |
| 19.4 | 33 |
| 20.6 | 100 |
| 21.4 | 31 |
| 22.1 | 38 |
| 22.4 | 10 |

According to one embodiment, the co-crystal of 8-Chloro-N-(4-(trifluoromethoxy)phenyl)quinolin-2-amine: L-Proline of the present invention presents a powder X-ray diffractogram displaying at least one peak expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least two peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least three peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least four peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least five peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least six peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least seven peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least eight peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least nine peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least ten peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least eleven peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least twelve peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least thirteen peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least fourteen peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention presents a powder X-ray diffractogram displaying at least fifteen peaks expressed as degree 2-Theta angle selected from 16.5; 20.6; 21.4; 22.1; 11.0; 15.9; 18.3; 19.4; 6.1; 12.2; 12.6; 13.3; 13.7; 15.4; 17.3 and 22.4 (each time ±0.2).

According to another embodiment, the co-crystal of 8-Chloro-N-(4-(trifluoromethoxy)phenyl)quinolin-2-amine: L-Proline of the present invention has an XRPD substantially similar to that depicted in FIG. 1.

A characteristic X-ray powder diffractogram of a co-crystal of 8-chloro-N-(4-(trifluoromethoxy)phenyl)quinolin-2-amine: Gentisic acid can be given in FIG. 2 and its characteristic signals are summarized in the following table 5:

TABLE 5

| Angle (2-Theta) | Relative intensity |
|---|---|
| (each time ±0.2) | (%) |
| 7.6 | 12 |
| 7.9 | 60 |
| 14.0 | 33 |
| 14.7 | 11 |
| 15.2 | 78 |
| 15.8 | 41 |
| 16.1 | 16 |
| 16.9 | 28 |
| 18.5 | 23 |
| 19.7 | 17 |
| 19.9 | 26 |
| 20.3 | 43 |
| 21.6 | 13 |
| 22.0 | 25 |
| 22.3 | 20 |
| 23.0 | 27 |
| 23.7 | 25 |
| 24.0 | 33 |
| 24.7 | 45 |
| 25.2 | 100 |

According to one embodiment, the co-crystal of 8-Chloro-N-(4-(trifluoromethoxy)phenyl)quinolin-2-amine: Gentisic acid of the present invention presents a powder X-ray diffractogram displaying at least one peak expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least two peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least three peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least four peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least five peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least six peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least seven peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least eight peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least nine peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least ten peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least eleven peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least twelve peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least thirteen peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least fourteen peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention presents a powder X-ray diffractogram displaying at least fifteen peaks expressed as degree 2-Theta angle selected from 7.9; 14.0; 15.2; 25.2; 15.8; 16.9; 18.5; 19.9; 20.3; 23.0; 24.7; 7.6; 14.7; 16.1; 19.7; 21.6; 22.0; 22.3; 23.7; and 24.0 (each time ±0.2).

According to another embodiment, the co-crystal of 8-Chloro-N-(4-(trifluoromethoxy)phenyl)quinolin-2-amine: Gentisic acid of the present invention has an XRPD substantially similar to that depicted in FIG. 2.

A characteristic X-ray powder diffractogram of a co-crystal of 8-chloro-N-(4-(trifluoromethoxy)phenyl)quinolin-2-amine: Malonic acid can be given in FIG. 3 and its characteristic signals are summarized in the following table 6:

TABLE 6

| Angle (2-Theta) | Relative intensity |
|---|---|
| (each time ±0.2) | (%) |
| 9.5 | 26 |
| 12.2 | 34 |
| 15.8 | 38 |
| 16.8 | 7 |
| 17.3 | 42 |
| 17.8 | 7 |
| 19.0 | 22 |
| 19.7 | 47 |
| 20.9 | 6 |
| 21.4 | 27 |
| 22.8 | 65 |
| 23.8 | 11 |
| 24.6 | 76 |
| 24.8 | 100 |
| 25.6 | 54 |
| 26.8 | 18 |
| 27.6 | 31 |
| 28.0 | 11 |
| 29.6 | 13 |
| 29.9 | 25 |

According to one embodiment, the co-crystal of 8-Chloro-N-(4-(trifluoromethoxy)phenyl)quinolin-2-amine: Malonic acid of the present invention presents a powder X-ray diffractogram displaying at least one peak expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least two peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least three peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least four peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least five peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least six peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least seven peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least eight peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least nine peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least ten peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least eleven peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least twelve peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least thirteen peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least fourteen peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention presents a powder X-ray diffractogram displaying at least fifteen peaks expressed as degree 2-Theta angle selected from 9.5; 12.2; 15.8; 17.3; 19.7; 22.8; 24.8; 25.6; 19.0; 21.4; 24.6; 26.8; 27.6; 29.9; 16.8; 17.8; 20.9; 23.8; 28.0; and 29.6 (each time ±0.2).

According to another embodiment, the co-crystal of 8-Chloro-N-(4-(trifluoromethoxy)phenyl)quinolin-2-amine: Malonic acid of the present invention has an XRPD substantially similar to that depicted in FIG. 3.

A characteristic X-ray powder diffractogram of a co-crystal of 8-chloro-N-(4-(trifluoromethoxy)phenyl)quinolin-2-amine: 4, 4′-Bipyridine can be given in FIG. 4 and its characteristic signals are summarized in the following table 7:

TABLE 7

| Angle (2-Theta) | Relative intensity |
|---|---|
| (each time ±0.2) | (%) |
| 8.5 | 5 |
| 12.0 | 16 |
| 13.0 | 7 |
| 15.7 | 7 |
| 16.0 | 12 |
| 16.7 | 9 |
| 17.0 | 30 |
| 17.8 | 16 |
| 19.2 | 40 |
| 20.3 | 14 |
| 20.9 | 11 |
| 21.2 | 100 |
| 22.0 | 8 |
| 22.5 | 21 |
| 22.7 | 21 |
| 23.1 | 10 |
| 23.6 | 12 |
| 24.3 | 36 |
| 24.7 | 10 |

According to one embodiment, the co-crystal of 8-chloro-N-(4-(trifluoromethoxy)phenyl)quinolin-2-amine: 4, 4′-Bipyridine of the present invention presents a powder X-ray diffractogram displaying at least one peak expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least two peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least three peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least four peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least five peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least six peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least seven peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least eight peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least nine peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least ten peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least eleven peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least twelve peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least thirteen peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least fourteen peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention presents a powder X-ray diffractogram displaying at least fifteen peaks expressed as degree 2-Theta angle selected from 12.0; 19.2; 21.2; 24.3; 16.0; 17.0; 17.8; 20.3; 22.5; 22.7; 8.5; 13.0; 15.7; 16.7; 20.9; 22.0; 23.1; 23.6 and 24.7 (each time ±0.2).

According to another embodiment, the co-crystal of 8-chloro-N-(4-(trifluoromethoxy)phenyl)quinolin-2-amine: 4, 4′-Bipyridine of the present invention has an XRPD substantially similar to that depicted in FIG. 4.

Method of Preparing Co-Crystals of ABX464

According to another aspect, the present invention further relates to a method of preparing a co-crystal of 8-chloro-N-(4-(trifluoromethoxy)phenyl)quinolin-2-amine as defined in the present invention comprising the following steps of:

a) dissolving ABX464 in a solvent or in a mixture of solvents;

b) adding to the thus obtained mixture of step a) a co-former which may be itself already dissolved in a solvent or in a mixture of solvents and which is selected from L-proline, gentisic acid, malonic acid and 4,4′-bipyridine so as to obtain a ABX464: co-former in molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent or a mixture of solvents,

e) applying a temperature program;

f) optionally filtrating; and

g) then optionally drying at a temperature comprised between room temperature and 60° C. in order to obtain the desired co-crystal of ABX464.

According to one embodiment, the solvent(s) used in step a), step b) and step d) is(are) any solvent conventionally used in crystallization step, particularly is(are) organic solvents, more particularly is(are) selected from a C₁-C₆ aliphatic alcohol, methyl ethyl ketone (also named butanone or MEK), cyclohexane, alkane such as heptane, methylene chloride, chloroform, formic acid, DMSO, 1-methyl-2-pyrrolidone, acetone, acetonitrile, tetrahydrofuran (THF), diethyl ether, dioxane, toluene, ethyl acetate, optionally in admixture with water, and mixtures thereof, still more particularly selected from a C₁-C₆ aliphatic alcohol, a mixture of H₂O/C₁-C₆ aliphatic alcohol, acetonitrile, and mixtures thereof, even more particularly selected from methanol, ethanol, isopropanol, H₂O/methanol, H₂O/ethanol, acetonitrile, and mixtures thereof.

The skilled person would know how to determine the more appropriate solvent(s) in each step a), b) and d) so as to obtain the desired co-crystal.

According to one embodiment, the solvent(s) used in step a), step b) and/or step d) is(are) the same.

According to another embodiment, the solvent(s) used in step a), step b) and/or step d) is(are) different.

According to one embodiment, the evaporating step c) is carried out under an inert gas such as N₂.

According to one embodiment the step e) relative to the temperature program includes a heating phase at reflux to a temperature comprised between room temperature and the boiling point of the solvent(s), particularly between room temperature and 60° C.

According to one embodiment the step e) relative to the temperature program includes a cooling phase at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min.

According to one embodiment, the step e) relative to the temperature program includes i) a heating at reflux to a temperature comprised between room temperature and the boiling point of the solvent(s), particularly between room temperature and 60° C. and/or ii) a cooling at reflux to a temperature comprised between 0° C. and 60° C., particularly between 5° C. and 40° C., more particularly between room temperature and 40° C. at a rate comprised between 30° C./min and 0.05° C./min, particularly between 10° C./min and 0.05° C./min, more particularly between 5° C./min and 0.05° C./min.

According to a particular embodiment, the step e) relative to the temperature program includes i) a heating at reflux to a temperature comprised between room temperature and 60° C. and ii) a cooling at reflux to a temperature comprised between 5° C. and 40° C., at a rate comprised between 30° C./min and 0.05° C./min, particularly between 10° C./min and 0.05° C./min, more particularly between 5° C./min and 0.05° C./min.

According to one embodiment, the step f) regarding the filtration is carried out using conventional glass fibers, conventional cellulosic filter papers, PTFE (polytetrafluoroethylene), or PVDF (polyvinylidene fluoride), particularly cellulosic filter paper with 0.45 μm or 0.2 μm filtration mesh.

According to one embodiment, the step g) regarding the drying is carried out under vacuum at a temperature comprised between 30° C. and 60° C., particularly at 40° C.

According to one embodiment, the step g) regarding the drying is carried out under ambient atmosphere at a temperature comprised between 30° C. and 60° C., particularly at 40° C.

According to a particular embodiment, the method of preparing a co-crystal of 8-chloro-N-(4-(trifluoromethoxy)phenyl)quinolin-2-amine as defined in the present invention comprises the following steps of:

a) dissolving ABX464 in a solvent or in a mixture of solvents;

b) adding to the thus obtained mixture of step a) a co-former which may be itself already dissolved in a solvent or in a mixture of solvents and which is selected from L-proline, gentisic acid, malonic acid and 4,4′-bipyridine so as to obtain a ABX464: co-former in molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent or a mixture of solvents,

e) applying a temperature program including i) a heating at reflux to a temperature comprised between room temperature and 60° C. and/or ii) a cooling at reflux to a temperature comprised between 5° C. and 40° C., at a rate comprised between 30° C./min and 0.05° C./min, particularly between 10° C./min and 0.05° C./min, more particularly between 5° C./min and 0.05° C./min;

f) optionally filtrating by using conventional glass fibers, conventional cellulosic filter papers, PTFE (polytetrafluoroethylene), or PVDF (polyvinylidene fluoride), particularly cellulosic filter paper with 0.45 μm or 0.2 μm filtration mesh; and

g) then optionally drying under vacuum at a temperature comprised between 30° C. and 60° C., particularly at 40° C. or under ambient atmosphere at a temperature comprised between 30° C. and 60° C., particularly at 40° C. in order to obtain the desired co-crystal of ABX464.

According to another particular embodiment, the method of preparing a co-crystal of 8-chloro-N-(4-(trifluoromethoxy)phenyl)quinolin-2-amine as defined in the present invention comprises the following steps of:

a) dissolving ABX464 in methanol, ethanol, isopropanol, H₂O/methanol, H₂O/ethanol, acetonitrile, or mixtures thereof;

b) adding to the thus obtained mixture of step a) a co-former which may be itself already dissolved in methanol, ethanol, isopropanol, H₂O/methanol, H₂O/ethanol, acetonitrile, or mixtures thereof and which is selected from L-proline, gentisic acid, malonic acid and 4,4′-bipyridine so as to obtain a ABX464: co-former in molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent which is selected from methanol, ethanol, isopropanol, H₂O/methanol, H₂O/ethanol, acetonitrile, and mixtures thereof,

e) applying a temperature program;

f) optionally filtrating; and

g) then optionally drying at a temperature comprised between room temperature and 60° C. in order to obtain the desired co-crystal of ABX464.

According to another particular embodiment, the method of preparing a co-crystal of 8-chloro-N-(4-(trifluoromethoxy)phenyl)quinolin-2-amine as defined in the present invention comprises the following steps of:

a) dissolving ABX464 in methanol, ethanol, isopropanol, H₂O/methanol, H₂O/ethanol, acetonitrile, or mixtures thereof;

b) adding to the thus obtained mixture of step a) a co-former which may be itself already dissolved in methanol, ethanol, isopropanol, H₂O/methanol, H₂O/ethanol, acetonitrile, or mixtures thereof and which is selected from L-proline, gentisic acid, malonic acid and 4,4′-bipyridine so as to obtain a ABX464: co-former in molar ratio comprised between 3:1 and 1:2, particularly between 5:2 and 1:2; more particularly between 2:1 and 1:2, and still more particularly is of 2:1 or 1:1;

c) optionally evaporating the solvent(s) at a temperature comprised between 0° C. and the boiling point of the selected solvent(s) or mixture of solvent(s) of step a) and step b), particularly between room temperature and 60° C., more particularly between room temperature and 50° C.;

d) optionally adding a solvent which is selected from methanol, ethanol, isopropanol, H₂O/methanol, H₂O/ethanol, acetonitrile, and mixtures thereof,

e) applying a temperature program including i) a heating at reflux to a temperature comprised between room temperature and 60° C. and/or ii) a cooling at reflux to a temperature comprised between 5° C. and 40° C., at a rate comprised between 30° C./min and 0.05° C./min, particularly between 10° C./min and 0.05° C./min, more particularly between 5° C./min and 0.05° C./min;

f) optionally filtrating by using conventional glass fibers, conventional cellulosic filter papers, PTFE (polytetrafluoroethylene), or PVDF (polyvinylidene fluoride), particularly cellulosic filter paper with 0.45 μm or 0.2 μm filtration mesh; and

g) then optionally drying under vacuum at a temperature comprised between 30° C. and 60° C., particularly at 40° C. or under ambient atmosphere at a temperature comprised between 30° C. and 60° C., particularly at 40° C. in order to obtain the desired co-crystal of ABX464.

According to another aspect, the present invention further relates to a method of preparing a co-crystal of 8-chloro-N-(4-(trifluoromethoxy)phenyl)quinolin-2-amine as defined in the present invention comprising the steps of:
a′) physically mixing 8-chloro-N-(4-(trifluoromethoxy)phenyl)quinolin-2-amine with a co-former chosen among L-Proline, Gentisic acid, Malonic acid and 4, 4′-Bipyridine at a molar ratio comprised between 2:1 and 1:2, particularly at a 1:1 molar ratio, in a suitable solvent or mixtures of solvents; and
b′) grinding of the physical mixture thus obtained from step a), in presence of one drop of solvent or of mixtures of solvents to obtain the co-crystal.

The skilled person would know how to determine the more appropriate solvent(s) in each step a′) and b′) so as to obtain the desired co-crystal.

According to one embodiment, the solvent(s) used in step a′) and step b′) is(are) any solvent conventionally used in crystallization step, particularly is(are) organic solvents, more particularly is(are) selected from a C₁-C₆ aliphatic alcohol, methyl ethyl ketone (also named butanone or MEK), cyclohexane, alkane such as heptane, methylene chloride, chloroform, formic acid, DMSO, 1-methyl-2-pyrrolidone, acetone, acetonitrile, tetrahydrofuran (THF), diethyl ether, dioxane, toluene, ethyl acetate, optionally in admixture with water, and mixtures thereof, still more particularly selected from a C₁-C₆ aliphatic alcohol, a mixture of H₂O/C₁-C₆ aliphatic alcohol, acetonitrile, and mixtures thereof, even more particularly selected from methanol, ethanol, isopropanol, H₂O/methanol, H₂O/ethanol, acetonitrile, and mixtures thereof.

In some embodiments, the grinding is carried out by using Retsch MM200 instrument for 45 min milling at 20 Hz.

According to one embodiment, the grinding is carried out by using non-oxidizable balls.

According to one embodiment, the grinding is carried out by using horizontal movements, particularly horizontal movements having a frequency ranging from 20 to 30 Hz.

Advantageously, the horizontal movements are applied for a period which may range from 15 minutes to 3 hours, particularly 45 min.

According to a particular embodiment, the present invention further relates to a method of preparing a co-crystal of 8-chloro-N-(4-(trifluoromethoxy)phenyl)quinolin-2-amine as defined in the present invention comprising the steps of:
a′) physically mixing 8-chloro-N-(4-(trifluoromethoxy)phenyl)quinolin-2-amine with a co-former chosen among L-Proline, Gentisic acid, Malonic acid and 4, 4′-Bipyridine at a molar ratio comprised between 2:1 and 1:2, particularly at a 1:1 molar ratio, in a suitable solvent which is selected from methanol, ethanol, isopropanol, H₂O/methanol, H₂O/ethanol, acetonitrile, and mixtures thereof; and
b′) grinding of the physical mixture thus obtained from step a), in presence of one drop of solvent which is selected from methanol, ethanol, isopropanol, H₂O/methanol, H₂O/ethanol, acetonitrile, and mixtures thereof to obtain the co-crystal.

It is understood that for the preparation of a co-crystal of ABX464, 8-chloro-N-(4-(trifluoromethoxy)phenyl)quinolin-2-amine can be previously obtained either from a process as the one described in WO2010/143169 or from any other appropriate process.

Co-Crystal of ABX 464, Pharmaceutically Acceptable Salts of ABX 464 and Pharmaceutical Compositions in Accordance with the Invention for Use as Medicines

According to another aspect, the present invention further relates to a pharmaceutical composition comprising as (an) active ingredient(s) at least one co-crystal of 8-chloro-N-(4-(trifluoromethoxy)phenyl)quinolin-2-amine as defined in the present invention and/or at least one pharmaceutically acceptable salts of 8-chloro-N-(4-(trifluoromethoxy)phenyl)quinolin-2-amine including a solvate and/or a hydrate thereof as defined in the present invention, and at least one pharmaceutically acceptable excipient.

According to another aspect, the present invention further relates to a pharmaceutical composition, wherein the pharmaceutical composition comprises at least one of the co-crystals as defined in the present invention and/or at least one of the pharmaceutically acceptable salts of 8-chloro-N-(4-(trifluoromethoxy)phenyl)quinolin-2-amine including a solvate and/or a hydrate thereof as defined in the present invention, as the sole pharmaceutically active ingredient(s).

Pharmaceutically acceptable compositions of the present invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes subcutaneous, intravenous, intramuscular, intra-articular, intra-synovial, intrasternal, intrathecal, intrahepatic, intralesional and intracranial injection or infusion techniques. In certain embodiments, the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof of the invention may be administered orally or parenterally at dosage levels of active ingredient ABX464 contained in the co-cristal or salt of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions such as aqueous solutions, suspensions such as aqueous suspensions, syrups and elixirs. In addition to the the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring and perfuming agents. When aqueous suspensions are required for oral use, the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention may be combined with emulsifying and suspending agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin amine including a solvate and/or a hydrate thereof as defined in the present invention with suitable non-irritating excipients or carriers such as cocoa butter, polyethylene glycol or a suppository wax which are solid at ambient temperature but liquid at body temperature and therefore melt in the rectum or vaginal cavity and release the active ABX464.

Solid dosage forms for oral administration include capsules, tablets, pills, powders, lozenges, chewing gums, and granules. In such solid dosage forms, the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention is mixed with at least one inert, pharmaceutically acceptable excipient or carrier such as sodium citrate or dicalcium phosphate and/or a) fillers or extenders such as starches such as corn starch, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. If desired, certain sweetening, flavoring or coloring agents may also be added.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular eight polethylene glycols and the like.

The co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention may be admixed with at least one inert diluent such as sucrose, lactose or starch.

Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Pharmaceutically acceptable compositions of the present invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be carried out in a rectal suppository formulation (see above) or in a suitable enema formulation. Topically transdermal patches may also be used.

For topical applications, provided pharmaceutically acceptable compositions may be formulated in a suitable ointment containing the active component suspended or dissolved in one or more carriers. Carriers for topical administration of the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers.

Suitable carriers include, but are not limited to, mineral oil, sorbitan monostearate, polysorbate 60, cetyl esters wax, cetearyl alcohol, 2-octyldodecanol, benzyl alcohol and water.

Additionally, the present invention contemplates the use of transdermal patches, which have the added advantage of providing controlled delivery of a compound to the body. Such dosage forms can be made by dissolving or dispensing the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention in the proper medium. Absorption enhancers can also be used to increase the flux of the compound across the skin. The rate can be controlled by either providing a rate controlling membrane or by dispersing the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention in a polymer matrix or gel.

Dosage forms for topical or transdermal administration of a co-crystal of 8-chloro-N-(4-(trifluoromethoxy)phenyl)quinolin-2-amine as defined in the present invention and/or a pharmaceutically acceptable salt of 8-chloro-N-(4-(trifluoromethoxy)phenyl)quinolin-2-amine including a solvate and/or a hydrate thereof as defined in the present invention include ointments, pastes, creams, lotions, gels, powders, solutions, sprays, inhalants or patches.

The co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention is admixed under sterile conditions with a pharmaceutically acceptable carrier and any needed preservatives or buffers as may be required.

Ophthalmic formulation, ear drops, and eye drops are also contemplated as being within the scope of the present invention.

Indeed, for ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of the present invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Most preferably, pharmaceutically acceptable compositions of the present invention are formulated for oral administration. Such formulations may be administered with or without food.

Thus, according to a particular embodiment, pharmaceutical compositions comprising the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention, and at least one pharmaceutically acceptable excipient, are in particular under the form of tablets, capsules, pills, lozenges, chewing gums, powders, granules, suppositories, emulsions, microemulsions, solutions such as aqueous solutions, suspensions such as aqueous suspensions, syrups, elixirs, ointments, drops, pastes, creams, lotions, gels, sprays, inhalants or patches.

In some embodiments, pharmaceutically acceptable compositions of the present invention are administered without food. In other embodiments, pharmaceutically acceptable compositions of the present invention are administered with food.

The amount of the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention that may be combined with the excipient or carrier materials to produce a composition in a single dosage form will vary depending upon the host treated, the particular mode of administration.

Among the pharmaceutical compositions according to the invention there may be mentioned, more especially, those that are suitable for oral, parenteral (intravenous or subcutaneous) or nasal administration, tablets or dragees, granules, sublingual tablets, capsules, lozenges, suppositories, creams, ointments, dermal gels, injectable preparations, drinkable suspensions and chewing gums.

According to a particular embodiment, a pharmaceutical composition in accordance with the invention is an oral pharmaceutical composition.

Oral pharmaceutical composition of the present invention can be in the form of capsules, tablets, or sachets comprising the composition in powder form. A therapeutically effective oral dosage for formulations of the invention is determined by standard clinical techniques according to the judgment of a medical practitioner.

According to one embodiment, when the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention are formulated into capsules, tablets, suspensions, solutions, or syrups by using conventional methods, they are protected in blisters. Another advantage conferred by the use of blisters is that the capsules or tablets are also protected from oxygen and other contaminants.

The capsules may be soft gel capsules or hard gel capsules. When the capsules are soft gel capsules, or hard gel capsules, they can advantageously comprise liquid excipients in particular:
Lipophilic liquid vehicles,
Semisolid lipophilic vehicles/viscosity modifiers for lipophilic liquid vehicles,
Solubilizing agents, surfactants, emulsifying agents and adsorption enhancers,

Among these excipients may be mentioned the following ones as shown below:
Refined specialty oils such as:
   Arachis oil
   Castor oil
   Cottonseed oil
   Maize (corn) oil
   Olive oil
   Sesame oil
   Soybean oil
   Sunflower oil
Medium-chain triglycerides and related esters such as:
   Caprylic/capric triglycerides (Akomed E, Akomed R, Miglyol 810, and Captex 355)
   Medium-chain triglyceride (Labrafac CC)
   Propylene glycol diester of caprylic/capric acid (Labrafac PG)
   Propylene glycol monolaurate (Lauroglycol FCC)
   Fractionated coconut oil (Miglyol 812)
   Caprylic/capric/diglyceryl succinate (Miglyol 829)
   Medium-chain diesters of propylene glycols (Miglyol 840)
   Partial ester of diglycerides with natural fatty acids (Softisan 645).
Solubilizing agents, surfactants, emulsifying agents, and adsorption enhancers such as:
   Propylene glycol monocaprylate (Capryol 90)
   Polyglycolized glycerides (Gelucire 44/14 and 50/13)
   Polyoxyl-40 hydrogenated castor oil (Cremophor RH 40)
   Glycerol monostearate/di-triglycerides+glycerin (Imwitor 191)
   Glyceryl monocaprylate (Imwitor 308*)
   Glyceryl cocoate/citrate/lactate (Imwitor 380)
   Glyceryl mono-di-caprylate/caprate (Imwitor 742)
   Isosteryl diglyceryl succinate (Imwitor 780 K)
   Glyceryl cocoate (Imwitor 928)
   Glyceryl caprylate (Imwitor 988)
   Oleoyl macrogol-8 glycerides (Labrafil M 1944 CS)
   Linoleoyl macrogolglycerides (Labrafil M 2125 CS)
   PEG-8 caprylic/capric glycerides (Labrasol)
   Lauric acid
   Propylene glycol laurate (Lauroglycol 90)
   Oleic acid
   Polyethylene glycol
   Propylene glycol
   Polyglycerol dioleate (Plurol Oleique CC 497)
   Polyoxyethylene-polyoxypropylene copolymer (Poloxamer 124 and 188)
   Partial glycerides of hydroxylated unsaturated fatty acids (Softigen 701)
   PEG-6 caprylic/capric glycerides (Softigen 767)
   Polyoxyethylene glyceryl trioleate (Tagat TO)
   Polyoxyethylene(20) sorbitan monooleate (Tween 80).

In some embodiments, the present invention provides a tablet or a capsule comprising the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention, and at least one pharmaceutically acceptable excipient.

In some particular embodiments, the present invention provides a capsule comprising the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin-2-amine including a solvate and/or a hydrate thereof as defined in the present invention, and at least one pharmaceutically acceptable excipient, or a tablet comprising granules formed by the co-crystal of 8-chloro-N-(4-(trifluoromethoxy)phenyl)quinolin-2-amine as defined in the present invention and/or the pharmaceutically acceptable salts of 8-chloro-N-(4-(trifluoromethoxy)phenyl)quinolin amine including a solvate and/or a hydrate thereof as defined in the present invention and by at least one intragranular excipient, said granules being compressed together with at least one extragranular excipient.

When the pharmaceutically composition in accordance with the invention is a tablet, the tablet may be coated or not coated. Preferably the tablet is coated by using any appropriate film-coating agent well known in the art.

Herein is in particular provided an oral galenic form comprising at least one co-crystal of 8-chloro-N-(4-(trifluoromethoxy)phenyl)quinolin-2-amine as defined in the present invention and/or at least one pharmaceutically acceptable salts of 8-chloro-N-(4-(trifluoromethoxy)phenyl)quinolin-2-amine including a solvate and/or a hydrate thereof as defined above and at least one inhibitor of precipitation as defined above.

Herein is further provided are coated oral dosage forms, in particular dedicated to delayed release dosage forms, comprising at least one co-crystal of 8-chloro-N-(4-(trifluoromethoxy)phenyl)quinolin-2-amine as defined above and/or at least one pharmaceutically acceptable salts of 8-chloro-N-(4-(trifluoromethoxy)phenyl)quinolin-2-amine including a solvate and/or a hydrate thereof as defined above.

The delayed release dosage forms are a particular modified dosage form allowing the release of the active principle from the dosage form after a particular time after administration, and for example once the active principle reaches the intestinal tract.

The term “modified release dosage form” means the dosage form allow a release into the body of a specified amount over a specified period of time of an active ingredient, namely a specific pharmacokinetic profile. The term “modified release” encompasses all the type of releases that are modified in comparison to an immediate release. In other words, the term “controlled release” is equivalent to “modified release” and encloses extended releases as well as delayed releases and pulse releases.

The oral dosage form may take the form of multi-particulate drug delivery systems or of multiple-layer compression-coated tablets. The coating may be chosen among polymers allowing the salt or co-crystal to rapidly solubilize once the dosage form reaches the biological target, and more particularly reaches the intestinal tract, and the polymer solubilizes.

Therefore, the present invention further provides the use of at least one co-crystal of 8-chloro-N-(4-(trifluoromethoxy)phenyl)quinolin-2-amine as defined above and/or at least one pharmaceutically acceptable salts of 8-chloro-N-(4-(trifluoromethoxy)phenyl)quinolin-2-amine including a solvate and/or a hydrate thereof as defined above for manufacturing a delayed release oral dosage form.

The useful dosage can be varied according to the nature and severity of the disorder, the administration route and the age and weight of the patient.

According to one embodiment, a pharmaceutical composition in accordance with the invention is such that a dose of from 1 mg to 1 g per day, particularly of from 10 mg to 150 mg per day of active ingredient ABX464 is administered to a subject in need thereof in one or more doses per day.

The excipients can be any conventional used excipients including intragranular excipients, and/or an extragranular excipients.

The excipients may be selected from fillers, glidants, binders, antioxidants, disintegrants, lubricants, surfactants, precipitation inhibitors, film-coating agents, and mixtures thereof.

Fillers which are useable in accordance with the invention include, but are not limited to, lactose (anhydrous), lactose monohydrate, spray-dried lactose; compressible sugar, dextrose, dextrates; starches (including starches from any source, such as corn, potato, rice, wheat, which can be fully pregelatinized and partially gelatinized); cellulose; microcrystalline cellulose; inorganic salts such as calcium phosphate, tribasic calcium and calcium sulfate; and polyols such as mannitol, sorbitol and xylitol.

In some embodiments, the filler may be in an amount of 10% to 85% by weight based on the total weight of the composition.

Lubricants which are useable according to the invention include, but are not limited to, magnesium stearate, calcium stearate, zinc stearate, stearic acid, sodium stearyl fumarate, hydrogenated vegetable oils, mineral oil, polyethylene glycols, talc, glyceryl behenate, glyceryl monostearate, glyceryl palmitostearate, leucine, and magnesium lauryl sulfate.

In some embodiments, the lubricant may be in an amount of 0.3% to 2% by weight based on the total weight of the composition.

Disintegrants which are useable in accordance with the invention include, but are not limited to, croscarmellose sodium, sodium starch glycolate, starches (including starches from any source, such as corn, potato, rice, wheat, fully pregelatinized and partially gelatinized), crospovidone, alginates such as calcium alginate and sodium alginate, alginic acid, and magnesium aluminum silicate.

In some embodiments, the disintegrant may be in an amount of 30% to 60% by weight based on the total weight of composition.

The surfactants employable as an additive in the present invention include, but are not limited to, sodium lauryl sulfate, tocopherol, lecithin, lauryl sulfate, Vitamin E, egg yolk phosphatides, docusate sodium, Capryol, Labrafil, Labrasol, Lauroglycol, Solutol (Macrogol-15 hydroxystearate), and mixtures thereof.

In some embodiments, the surfactant may be in an amount of 1% to 3% by weight based on the total weight of composition.

Glidants which are useable in accordance with the invention include, but are not limited to colloidal silicon dioxide.

In some embodiments, the glidant may be in an amount of 0.3% to 2% by weight based on the total weight of composition.

In some embodiments, the binder may be in an amount of 5% to 20% by weight based on the total weight of composition.

The pharmaceutical compositions according to the present invention may be under modified, sustained, controlled, delayed, or immediate release form.

According to another aspect, the present invention also relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use as a medicine.

According to another aspect, the present invention also relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in the prevention and/or treatment of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia.

According to another aspect, the present invention also relates to the use of a co-crystal of 8-chloro-N-(4-(trifluoromethoxy)phenyl)quinolin-2-amine as defined in the present invention, of a pharmaceutically acceptable salt of 8-chloro-N-(4-(trifluoromethoxy)phenyl)quinolin-2-amine including a solvate and/or a hydrate thereof as defined in the present invention, or of a pharmaceutical composition as defined in the present invention for the manufacture of a medicament.

According to another aspect, the present invention also relates to the use of a co-crystal of 8-chloro-N-(4-(trifluoromethoxy)phenyl)quinolin-2-amine as defined in the present invention, of a pharmaceutically acceptable salt of 8-chloro-N-(4-(trifluoromethoxy)phenyl)quinolin-2-amine including a solvate and/or a hydrate thereof as defined in the present invention, or of a pharmaceutical composition as defined in the present invention for the manufacture of a medicament for preventing and/or treating of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia.

According to another aspect, the present invention also relates to a therapeutic method of treating and/or preventing of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia comprising administering to a patient in need thereof a composition comprising a co-crystal of 8-chloro-N-(4-(trifluoromethoxy)phenyl)quinolin-2-amine as defined in the present invention and/or a pharmaceutically acceptable salt of 8-chloro-N-(4-(trifluoromethoxy)phenyl)quinolin-2-amine including a solvate and/or a hydrate thereof as defined in the present invention.

According to another aspect, the present invention also relates to a therapeutic method of treating and/or preventing of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia comprising administering to a patient in need thereof a therapeutically effective amount of a co-crystal of 8-chloro-N-(4-(trifluoromethoxy)phenyl)quinolin-2-amine as defined in the present invention and/or a pharmaceutically acceptable salt of 8-chloro-N-(4-(trifluoromethoxy)phenyl)quinolin-2-amine including a solvate and/or a hydrate thereof as defined in the present invention.

A method for administering a co-crystal of ABX464 to a subject in need thereof is provided, comprising:
providing an oral pharmaceutical composition comprising: a co-crystal of ABX464 and/or a pharmaceutically acceptable salt of 8-chloro-N-(4-(trifluoromethoxy)phenyl)quinolin-2-amine including a solvate and/or a hydrate thereof as defined in the present invention, and at least one pharmaceutically acceptable excipient; and
orally administering the pharmaceutical composition in a therapeutically effective amount to a subject in need thereof.

A co-crystal of ABX464 as defined in the present invention can be administered alone or in combination with other therapeutic agents which can act synergistically with said co-crystal of ABX464 as defined in the present invention. For example, another therapeutic agent can be a pharmaceutically acceptable salt of 8-chloro-N-(4-(trifluoromethoxy)phenyl)quinolin-2-amine including a solvate and/or a hydrate thereof as defined in the present invention.

By analogy, a pharmaceutically acceptable salt of 8-chloro-N-(4-(trifluoromethoxy)phenyl)quinolin-2-amine including a solvate and/or a hydrate thereof as defined in the present invention can be administered alone or in combination with other therapeutic agents which can act synergistically with said pharmaceutically acceptable salt of ABX464 including a solvate and/or a hydrate thereof as defined in the present invention. For example, another therapeutic agent can be a co-crystal of ABX464 as defined in the present invention.

Inflammatory Diseases

Thus, the invention also relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention, for use in the treatment and/or prevention of an inflammatory disease.

According to the invention, an «inflammation» is a protective response by the immune system to tissue damage and infection. However, the inflammatory response, in some circumstances, can damage the body. In the acute phase, inflammation is characterized by pain, heat, redness, swelling and loss of function Inflammation can result from infection, irritation, or injury.

Thus, an «inflammatory disease» refers to a group of diseases and/or disorders that are caused by an excessive or dysregulated inflammation.

In a non-limitative manner, inflammatory diseases include: an inflammatory disease associated with an autoimmune disease, a central nervous system (CNS) inflammatory disease, a joint inflammation disease, an inflammatory digestive tract disease, inflammatory skin and other inflammatory diseases related to epithelial cells such as bronchitis, inflammation associated with cancer, such as colon carcinoma, inflammation associated with irritation, and inflammation associated with injury.

According to the present invention, the inflammatory disease, disorder or condition is selected from:
(a) an inflammatory disease, disorder, or condition in the pancreas selected from diabetes type-1, diabetes type-2, acute and chronic pancreatitis;
(b) an inflammatory disease, disorder, or condition in the kidney selected from glomerulosclerosis, glomerulonephritis, nephritis, acute kidney injury, Berger's disease, Goodpasture's syndrome, Wegener's granulomatosis and kidney transplant acute or chronic rejection;
(c) an inflammatory disease, disorder, or condition in the liver selected from nonalcoholic steatohepatitis (NASH), non-alcoholic fatty liver disease (NAFLD), cholestatic liver disease, sclerosing cholangitis and liver transplant acute or chronic rejection;
(d) an inflammatory disease, disorder, or condition in the lung or heart selected from chronic obstructive pulmonary disease (COPD), asthma, pulmonary fibrosis, pulmonary arterial hypertension, sarcoidosis, myocarditis, pericarditis and lung or heart transplant acute or chronic rejection;
(e) an inflammatory disease, disorder, or condition in the skin selected from contact dermatitits, atopic dermatitis, urticaria, chronic dermatitis, psoriasis, eczema, alopecia areata, erythema multiforma, dermatitis herpetiformis, scleroderma, vitiligo, hypersensitivity angiitis, urticaria, bullous pemphigoid, pemphigus vulgaris, pemphigus foliaceus, paraneoplastic pemphigus, epidermolysis bullosa acquisita, acnea, keloid scar, and other inflammatory or allergic conditions of the skin;
(f) an inflammatory disease, disorder, or condition in the vessel/blood selected from Behcet's disease, vasculitis, sepsis, tumor angiogenesis, atherosclerosis, proliferative vascular disease and restenosis;
(g) an inflammatory disease, disorder, or condition in the eye selected from conjunctivitis, scleritis, episcleritis, panuveitis, choroiditis, chorioretinitis, neuroretinitis, uveitis, orbital inflammatory disease, and optical neuritis;
(h) an inflammatory disease, disorder, or condition in the central or peripheral nervous system selected from non-viral and viral encephalitis and meningitis, depression, neuropathic pain, chronic pain, traumatic brain injury, including stroke, Alzheimer disease, Parkinson disease, Myelitis, Charcot-Marie-Tooth disease type 1 (including CMT1A and CMT1B), Multiple Sclerosis, Amyotrophic lateral sclerosis (ALS), Creutzfeldt-Jakob disease, demyelinating polyneuropathy and peripheral neuropathy;
(i) an autoimmune disease, disorder, or condition selected from Lupus, including in the skin and kidney, Guillain-Barre syndrome, Myasthenia gravis, Hashimoto's thyroiditis, idiopathic purpura, aplastic anemia, Graves disease, and Myocarditis;
(j) an inflammatory disease, disorder, or condition in the intestine selected from intestinal failure, Ulcerative colitis (UC) and Crohn's disease,
(k) an inflammatory disease, disorder, or condition in the reproductive system selected from endometriosis, uterine fibroma, prostate dysplasia or growth, and cervix dysplasia; and
(l) an inflammatory disease, disorder, or condition in the bone and/or joints selected from rheumatoid arthritis, juvenile idiopathic arthritis, psoriatic arthritis, periodontitis, and hand, foot, ankle, knee, hip, shoulder, elbow or spine arthritis and/or demineralization.

In a particular embodiment, the inflammatory disease can be selected in the list consisting of: an inflammatory disease associated with an autoimmune disease, a central nervous system (CNS) inflammatory disease, a joint inflammation disease, an inflammatory digestive tract disease, inflammatory skin and other inflammatory diseases related to epithelial cells, inflammation associated with cancer, inflammation associated with irritation, and inflammation associated with injury.

In particular, an inflammatory disease is selected in the list consisting of: Inflammatory Bowel Disease, Rheumatoid Arthritis, Crohn's disease, Ulcerative Colitis, Multiple Sclerosis, Alzheimer's disease, Parkinson, osteoarthritis, atherosclerosis, ankylosing spondylitis, psoriasis, dermatitis, Sjogren's syndrom, bronchitis, asthma, pulmonary arterial hypertension, NASH and inflammation associated with colon carcinoma.

More particularly, an inflammatory disease is selected in the list consisting of: Inflammatory Bowel Disease, Rheumatoid Arthritis, Crohn's disease, Ulcerative Colitis, Multiple Sclerosis, osteoarthritis, ankylosing spondylitis, psoriasis, Sjogren's syndrom, bronchitis, pulmonary arterial hypertension, NASH and inflammation associated with colon carcinoma.

More particularly, an inflammatory disease is selected in the list consisting of: Inflammatory Bowel Disease, Rheumatoid Arthritis, Crohn's disease, Ulcerative Colitis, Multiple Sclerosis, osteoarthritis, ankylosing spondylitis, pulmonary arterial hypertension, NASH and psoriasis.

Preferably, an inflammatory disease according to the invention includes: Inflammatory Bowel Disease, Crohn's disease, Ulcerative Colitis, Rheumatoid Arthritis, pulmonary arterial hypertension, NASH and Multiple Sclerosis.

Even more preferably, an inflammatory disease according to the invention includes: Inflammatory Bowel Disease, Rheumatoid Arthritis, pulmonary arterial hypertension, NASH and Multiple Sclerosis.

An inflammatory disease may also encompass Alzheimer's disease, Parkinson, asthma, atherosclerosis and dermatitis.

As dermatitis, eczema may be cited.

In view of the above, the invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention for use in the treatment and/or prevention of an inflammatory disease, which encompasses inflammation as such, and inflammation associated with an inflammatory disease.

Thus, the invention also relates to the use of a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for treating and/or preventing an inflammatory disease, which encompasses inflammation as such, and inflammation associated with an inflammatory disease.

The invention also relates to the use of a co-crystal of 8-chloro-N-(4-(trifluoromethoxy)phenyl)quinolin-2-amine as defined in the present invention, or a pharmaceutically acceptable salt of 8-chloro-N-(4-(trifluoromethoxy)phenyl)quinolin-2-amine including a solvate and/or a hydrate thereof as defined in the present invention, for the preparation of a composition, such as a medicament, for treating and/or preventing inflammation, which encompasses inflammation as such, and inflammation associated with an inflammatory disease.

The invention also relates to a method for treating and/or preventing an inflammatory disease, which includes inflammation as such, and inflammation associated with said inflammatory disease, and which comprises a step of administering a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention to a patient in need thereof.

In some embodiments, a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention for use as defined above, for treating an inflammatory disease, disorder or condition further comprises measuring and/or monitoring a presence and/or level of a biomarker in a patient, for example in a blood, plasma, tissue, saliva, and/or serum sample. In some embodiments, a biomarker measured and/or monitored in a method of the present invention is miR-124.

In some embodiments, a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating an inflammatory disease, disorder or condition further comprises measuring and/or monitoring a presence and/or expression level of miR-124 in a patient, for example in a blood, plasma, tissue, saliva, and/or serum sample, prior to administering a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention as described herein.

In some embodiments, a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating an inflammatory disease, disorder or condition further comprises measuring and/or monitoring a presence and/or expression level of miR-124 in a patient during the course of a treatment with a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention thereof as described herein.

In some embodiments, a method of the present invention for treating an inflammatory disease, disorder or condition, or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, further comprises selecting a patient for a treatment with a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention thereof as described herein, by measuring and/or monitoring a presence and/or expression level of miR-124 in the patient.

A provided co-crystal of 8-chloro-N-(4-(trifluoromethoxy)phenyl)quinolin-2-amine as defined in the present invention, or a provided pharmaceutically acceptable salt of 8-chloro-N-(4-(trifluoromethoxy)phenyl)quinolin-2-amine including a solvate and/or a hydrate thereof as defined in the present invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from a provided co-crystal of 8-chloro-N-(4-(trifluoromethoxy)phenyl)quinolin-2-amine as defined in the present invention, or from a provided pharmaceutically acceptable salt of 8-chloro-N-(4-(trifluoromethoxy)phenyl)quinolin-2-amine including a solvate and/or a hydrate thereof as defined in the present invention, as part of a multiple dosage regimen. Alternatively, those agents may be part of a single dosage form, mixed together with a provided co-crystal of 8-chloro-N-(4-(trifluoromethoxy)phenyl)quinolin-2-amine as defined in the present invention, or with a provided pharmaceutically acceptable salt of 8-chloro-N-(4-(trifluoromethoxy)phenyl)quinolin-2-amine including a solvate and/or a hydrate thereof as defined in the present invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a provided co-crystal of 8-chloro-N-(4-(trifluoromethoxy)phenyl)quinolin-2-amine as defined in the present invention, or a provided pharmaceutically acceptable salt of 8-chloro-N-(4-(trifluoromethoxy)phenyl)quinolin-2-amine including a solvate and/or a hydrate thereof as defined in the present invention may be administered with another therapeutic agent simultaneously or sequentially in separate unit dosage forms or together in a single unit dosage form. Accordingly, the present invention provides a single unit dosage form comprising a provided co-crystal of 8-chloro-N-(4-(trifluoromethoxy)phenyl)quinolin-2-amine as defined in the present invention, or a provided pharmaceutically acceptable salt of 8-chloro-N-(4-(trifluoromethoxy)phenyl)quinolin-2-amine including a solvate and/or a hydrate thereof as defined in the present invention, an additional therapeutic agent, and a pharmaceutically acceptable carrier, adjuvant, or vehicle.

In some embodiments, the co-crystal of 8-chloro-N-(4-(trifluoromethoxy)phenyl)quinolin-2-amine as defined in the present invention, or the pharmaceutically acceptable salt of 8-chloro-N-(4-(trifluoromethoxy)phenyl)quinolin-2-amine including a solvate and/or a hydrate thereof as defined in the present invention may be administered with one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®, Neoral®), tacrolimus, sirolimus, mycophenolate, leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), anti-T cell antibodies such as Thymoglobulin, IV Immunoglobulins (IVIg), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), anti-IL36 agents such as BI655130, Dihydroorotate dehydrogenase inhibitors such as IMU-838, anti-OX40 agents such as KHK-4083, microbiome agents such as RBX2660, SER-287, Narrow spectrum kinase inhibitors such as TOP-1288, anti-CD40 agents such as BI-655064 and FFP-104, guanylate cyclase agonists such as dolcanatide, sphingosine kinase inhibitors such as opaganib, anti-IL-12/IL-23 agents such as AK-101, Ubiquitin protein ligase complex inhibitors such as BBT-401, sphingosine receptors modulators such as BMS-986166, P38MAPK/PDE4 inhibitors such as CBS-3595, CCR9 antagonists such as CCX-507, FimH antagonists such as EB-8018, HIF-PH inhibitors such as FG-6874, HIF-1α stabilizer such as GB-004, MAP3K8 protein inhibitors such as GS-4875, LAG-3 antibdies such as GSK-2831781, RIP2 kinase inhibitors such as GSK-2983559, Farnesoid X receptor agonist such as MET-409, CCK2 antagonists such as PNB-001, IL-23 Receptor antagonists such as PTG-200, Purinergic P2X7 receptor antagonists such as SGM-1019, PDE4 inhibiotrs such as Apremilast, ICAM-1 inhibitors such as alicaforsen sodium, Anti-IL23 agents such as guselkumab, brazikumab and mirkizumab, ant-IL-15 agents such as AMG-714, TYK-2 inhibitors such as BMS-986165, NK Cells activators such as CNDO-201, RIP-1 kinase inhibitors such as GSK-2982772, anti-NKGD2 agents such as JNJ-4500, CXCL-10 antibodies such as JT-02, IL-22 receptor agonists such as RG-7880, GATA-3 antagonists such as SB-012 and Colony-stimulating factor-1 receptor inhibitors such as edicotinib or any combination(s) thereof.

Diseases Caused by Viruses

A co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, may be useful in the treatment or prevention of various diseases caused by viruses, in particular by retroviruses and more particularly by HIV, and more particularly for use for lowering viral load in a patient infected by a virus, in particular HIV, or a virus-related condition, with a long-lasting effect and absence of resistance.

Examples of viruses which are considered by the invention include enveloped and naked viruses, which includes DNA viruses, RNA viruses and retroviruses, which includes dsDNA viruses, ssDNA viruses, dsRNA viruses, (+)ssRNA viruses, (−)ssRNA viruses, ssRNA-RT viruses and dsDNA-RT viruses.

Viruses which are more particularly considered are RNA viruses and retroviruses, including lentiviruses, and preferably HIV. Accordingly, virus-related conditions which are more particularly considered are associated with a RNA virus or a retrovirus, and preferably HIV. HIV may include HIV-I, HIV-2 and all subtypes thereof, which includes HIV-I strains belonging to the HIV-I B subtype, HIV-I C subtype, and HIV-I recombinants. Examples include HIV-I strains selected from Ad8, AdaM, Isolate B, Isolate C, CRF01, CRFO2 and CRF06. According to a preferred embodiment, the virus-related condition is AIDS.

Three subfamilies can be distinguished within the retroviral family: the oncoviruses, the lentiviruses and the spumaviruses. HIV pertains to the lentiviruses.

According to a particular embodiment, the retroviruses include HIV virus (HIV1 and HIV2), visna/maedi virus or MVV/visna, equine infectious anemia virus or EIAV, caprine arthritis encephalitis virus or CAEV, simian immunodeficiency virus or SIV, avian leukemia virus or ALV, murine leukemia virus also called Moloney virus or MULV, Abelson leukemia virus, murine mammary tumor virus, Mason-Pfizer monkey virus or MPMV, feline leukemia virus or FELV, human leukemia viruses HTLV-I, human leukemia viruses HTLV-II, simian leukemia virus or STLV, bovine leukemia virus or BLV, primate type D oncoviruses, type B oncoviruses, Rous sarcoma virus or RSV, simian foamy virus or SFV or chimpanzee simian virus, human foamy virus, and feline immunodeficiency virus, the human foamy virus or HFV, bovine syncytial virus or BSV, feline syncytial virus FSV, the feline immunodeficiency virus, avian leukosis virus, Walleye dermal sarcoma virus, T-cell lymphoma, acute ATL, lymphomatous ATL, chronic ATL, smoldering ATL, neurologic diseases, Tropical spastic paraparesis or HTLV-associated myelopathy, inflammatory and autoimmune diseases such as uveitis, dermatitis, pneumonitis, rheumatoid arthritis, and polymyositis hematologic and dermatologic diseases, lung diseases, brain diseases, and/or immunodeficiency.

As described herein, the term oncovirus can include Alpharetrovirus (for example, avian leukosis virus and Rous sarcoma virus); Betaretrovirus (for example, mouse mammary tumor virus); Gammaretrovirus (for example, murine leukemia virus and feline leukemia virus); Deltaretrovirus (for example bovine leukemia virus and human T-lymphotropic virus); and Epsilonretrovirus (for example, Walleye dermal sarcoma virus).

More generally, the retroviruses described herein may be, for example, visna/maedi virus or MVV/visna, equine infectious anemia virus or EIAV, caprine arthritis encephalitis virus or CAEV, simian immunodeficiency virus or SIV, avian leukemia virus or ALV, murine leukemia virus also called Moloney virus or MULV, Abelson leukemia virus, murine mammary tumor virus, Mason-Pfizer monkey virus or MPMV, feline leukemia virus or FELV, human leukemia viruses HTLV-I, human leukemia viruses HTLV-II, simian leukemia virus or STLV, the bovine leukemia virus or BLV, primate type D oncoviruses, type B oncoviruses, Rous sarcoma virus or RSV, and/or simian foamy virus or SFV or chimpanzee simian virus, human foamy virus, and feline immunodeficiency virus, the human foamy virus (or HFV), bovine syncytial virus (or BSV), feline syncytial virus (FSV) and the feline immunodeficiency virus.

More particularly, HTLV-I causes T-cell lymphoma (ATL for Adult T-cell leukemia/lymphoma, including the different forms of ATL such as acute ATL, lymphomatous ATL, chronic ATL and smoldering ATL), neurologic disease, Tropical spastic paraparesis (TSP) (also known as HTLV-associated myelopathy (HAM) or chronic progressive myelopathy), and diverse inflammatory and autoimmune diseases such as uveitis, dermatitis, pneumonitis, rheumatoid arthritis, and; HTLV-II may play a role in certain neurologic, hematologic and dermatologic diseases; HIV (HIV1 and HIV2) causes AIDS; the visna virus causes lung and brain diseases in sheep; the feline immunodeficiency virus causes immunodeficiency in cat; Rous sarcoma virus and mouse mammary tumor virus causes tumor growth and cancer.

The invention also relates to a method for treating and/or preventing diseases caused by viruses, in particular by retroviruses and more particularly by HIV, and which comprises a step of administering a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention to a patient in need thereof.

Additionally, the present invention has for purpose to lower a viral load in a patient infected by a virus, in particular HIV, or a virus-related condition, with a long-lasting effect and absence of resistance, by using a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention.

In one embodiment, the present invention concerns a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use for treating or preventing a retroviral infection or a retrovirus-related condition, in particular a HIV infection or a HIV-related condition in a patient, for which an ineffectiveness or a decline in a prior anti-retroviral treatment effectiveness has been stated.

In another embodiment, the present invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use for treating or preventing a retroviral infection or a retrovirus-related condition, in particular a HIV infection or a HIV-related condition in a patient, wherein the patient is infected by a drug-resistant viral strain, and more particularly by a drug-resistant HIV strain.

Furthermore, the invention further relates to new doses and regimens of said co-crystal of 8-chloro-N-(4-(trifluoromethoxy)phenyl)quinolin-2-amine as defined in the present invention, or said pharmaceutically acceptable salt of 8-chloro-N-(4-(trifluoromethoxy)phenyl)quinolin-2-amine including a solvate and/or a hydrate thereof as defined in the present invention and use in the treatment or prevention of viral infection, and in particular HIV, or a virus-related condition, more particularly where the use maintains a low viral load after treatment termination. Thus, according to one embodiment, the invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use for treating or preventing of a virus infection or virus-related condition in a patient, in particular a HIV infection or a HIV-related condition, wherein: a low or undetectable viral load is maintained; and/or a CD4+ cell count is stable or increased; after treatment termination.

According to another embodiment, the invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in the treatment or prevention of a virus infection or virus-related condition in patient, in particular a HIV infection or a HIV-related condition, for which an ineffectiveness in prior anti-retroviral treatment, or a decline in a prior anti-viral, or anti-retroviral, treatment effectiveness has been stated.

According to still another embodiment, the invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in the treatment or prevention of a virus infection or virus-related condition in patient, in particular a HIV infection or a HIV-related condition, wherein the patient is infected by a drug-resistant strain.

In the framework of the present invention, the co-crystal of 8-chloro-N-(4-(trifluoromethoxy)phenyl)quinolin-2-amine as defined in the present invention, or the pharmaceutically acceptable salt of 8-chloro-N-(4-(trifluoromethoxy)phenyl)quinolin amine including a solvate and/or a hydrate thereof as defined in the present invention may be administered in combination with another anti-retroviral agent. According to one embodiment, an ART (Antiretroviral Therapy) or HAART (Highly Active Antiretroviral Therapy) may be implemented using one or more of the following antiretroviral compounds:

(i) nucleoside/nucleotide reverse transcriptase inhibitors also called nucleoside analogs, such as abacavir, emtricitabine, and tenofovir;

(ii) non-nucleoside reverse transcriptase inhibitors (NNRTIs), such as efavirenz, etravirine, and nevirapine;

(iii) protease inhibitors (PIs), such as atazanavir, darunavir, and ritonavir;

(iv) entry inhibitors, such as enfuvirtide and maraviroc;

(v) integrase inhibitors, such as dolutegravir and raltegravir.

Other examples of anti-retroviral agents include, in a non-limitative manner: Zidovudine, Lamivudine, Emtricitabine, Didanosine, Stavudine, Abacavir, Zalcitabine, Racivir, Amdoxovir, Apricitabine, Elvucitabine, Efavirenz, Nevirapine, Etravirine, Delavirdine, Rilpvirine, Tenofovir, Fosalvudine, Amprenavir, Tipranavir, Indinavir, Saquinavir, Fosamprenavir, Ritonavir, Darunavir, Atazanavir, Nelfinavir, Lopinavir, Raltegravir, Elvitegravir, Dolutegravir, Enfuvirtide, Maraviroc, Vicriviroc, and combinations thereof.

In some embodiments, a co-crystal of 8-chloro-N-(4-(trifluoromethoxy)phenyl)quinolin-2-amine as defined in the present invention, or a pharmaceutically acceptable salt of 8-chloro-N-(4-(trifluoromethoxy)phenyl)quinolin-2-amine including a solvate and/or a hydrate thereof as defined in the present invention may be administered in combination with one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

A co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, may also be useful in the treatment and/or prevention of diseases caused by a virus belonging to Coronaviridae family or by a Coronaviridae infection and conditions related thereto, and especially the Severe Acute Respiratory Syndrome caused by SARS-CoV or SARS-CoV-2 infection including strains responsible for COVID-19 (also referred herein as coronavirus disease 2019) and their mutants.
More particularly, SARS-CoV-2, previously known as 2019-nCoV belongs to the Coronaviridae family and does part of the group IV of the Baltimore classification. For reference, the content of the “Baltimore classification” which is reported herein further references to the virus taxonomy as set forth in the database of the International Committee of Taxonomy of Viruses (ICTV) as available online on Mar. 20, 2020 (Email ratification February 2019 & MSL #34) at https://talk.ictvonline.org/taxonomy/. This taxonomy is incorporated herein in its entirety.

Accordingly, this classification clusters viruses into families (or “groups”) depending on their type of genome. The present virus classification, as in 2018, comprises seven different groups:
Group I: double-stranded DNA viruses (dsDNA);
Group II: single-stranded DNA viruses (ssDNA);
Group III: double-stranded RNA viruses (dsRNA);
Group IV: (+)strand or sense RNA viruses ((+)ssRNA);
Group V: (−)strand or antisense RNA viruses ((−)ssRNA);
Group VI: single-stranded RNA viruses having DNA intermediates (ssRNA-RT);
Group VII: double-stranded DNA viruses having RNA intermediates (dsDNA-RT).
   A co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention are moreover particularly useful for treating and/or preventing severe forms of SARS-CoV-2 infections: anti-inflammatory effects to fight the cytokine storm, mucosal effectiveness, promotion of tissue repair to avoid long-term post-ventilation sequelae.

According to a particular embodiment, a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention may be used at early stage of the COVID-19.

Indeed, clinically, SARS-CoV-2 infection can lead to a cytokine storm syndrome, acute respiratory distress syndrome (ARDS) and multiple organ failure. Notably, cytokine storm (i.e. hyperinflammatory syndrome) has been associated with COVID-19 disease severity (including increased MCP1, IL-1β, TNFα, IL-17, G-CSF and IL-6). Early treatment and acting on viral replication and on the various cytokine pathways allow to successfully reduce the cytokine storm syndrome and “hyper-inflammation” and to prevent ARDS and multi-organ failure.

Accordingly, in one embodiment, the present invention relates to A co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in a method for treating a group of patients prior to the occurrence of a respiratory distress syndrome related to a Coronaviridae infection. Said patients may or not be hospitalized.

Accordingly, in one embodiment, the present invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in a method for treating or preventing the occurrence of a respiratory distress syndrome related to a Coronaviridae infection.

According to particular embodiments, a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention, is for use in a method for treating or preventing a Coronaviridae infection, is for treating or preventing the occurrence of a vascular, a cardiovascular, a neurological or a gastrointestinal condition related to a Coronaviridae infection.

Advantageously, a co-crystal of 8-chloro-N-(4-(trifluoromethoxy)phenyl)quinolin-2-amine as defined in the present invention, or a pharmaceutically acceptable salt of 8-chloro-N-(4-(trifluoromethoxy)phenyl)quinolin-2-amine including a solvate and/or a hydrate thereof as defined in the present invention, may be considered either alone or in combination with any other active agent, in particular any dynamin inhibitor, especially any dynamin-2 inhibitor, for use in the treatment of prevention of a Coronaviridae infection.

As used herein, a “condition related to a Coronaviridae infection”, especially a condition related to a Severe acute respiratory syndrome-related coronavirus, such as SARS-CoV2, may be selected from a list comprising, or consisting of: severe respiratory distress syndrome, a cardiovascular condition, a vascular condition, a gastrointestinal condition or a neurological condition.

Advantageously, the patients having, or being at risk of a having a condition related to a Coronaviridae infection can also be considered.

According to exemplary embodiments, the condition related to a Coronaviridae infection which are particularly considered include: pulmonary fibrosis, vasculitis, Kawasaki disease and tissue damage or destruction, in particular lung tissue damage and destruction.

Unless instructed otherwise, all the disclosed co-crystals of 8-chloro-N-(4-(trifluoromethoxy)phenyl)quinolin-2-amine as defined in the present invention, pharmaceutically acceptable salts of 8-chloro-N-(4-(trifluoromethoxy)phenyl)quinolin amine including a solvate and/or a hydrate thereof as defined in the present invention, and pharmaceutical compositions as defined in the present invention are specifically considered herein for the treatment or prevention of Coronaviridae, which may thus refer indifferently to any member of the said Coronaviridae family in the sense of the Baltimore convention, although particular selections of viruses will be considered hereafter as preferred embodiments.

As used herein, the term “Coronaviridae” refers to the corresponding family of RNA viruses belonging to the group IV of the Baltimore classification, which is it iself par of the Coronidovirineae suborder and of the Nidovirales Order. The Coronaviridae family includes both the Letovirinae and Orthocoronavirinae subfamilies.

As used herein, the term “Letovirinae” refers to the corresponding family of the Baltimore classification, which includes the Alphaletovirus genus, the Milecovirus subgenus, which includes (in a non-exhaustive manner) the Microhyla letovirus 1 species.

As used herein, the term “Orthocoronavirinae” refers to the corresponding family of the Baltimore classification, which includes the Alphacoronavirus, Betacoronavirus, Deltacoronavirus, and Gammacoronavirus genus.

As used herein, the term “Alphacoronavirus” refers to the corresponding family of the Baltimore classification, which includes the Colacovirus, Decacovirus, Duvinacovirus, Luchacovirus, Minacovirus, Minunacovirus, Myotacovirus, Myctacovirus, Pedacovirus, Rhinacovirus, Setracovirus, and Tegacovirus subgenus. In a non-exhaustive manner, this includes the following species: Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Alphacoronavirus 1.

As used herein, the term “Betacoronavirus” refers to the corresponding family of the Baltimore classification, which includes the Embecovirus, Hibecovirus, Merbecovirus, Nobecovirus, and Sarbecovirus subgenus. In a non-exhaustive manner, this includes the following species: Betacoronavirus 1, China Rattus coronavirus HKU24, Human coronavirus HKU1, Murine coronavirus, Bat Hp-betacoronavirus Zhejiang2013, Hedgehog coronavirus 1, Middle East respiratory syndrome-related coronavirus, Pipistrellus bat coronavirus HKU5, Tylonycteris bat coronavirus HKU4, Hedgehog coronavirus 1, Middle East respiratory syndrome-related coronavirus, Pipistrellus bat coronavirus HKU5, Tylonycteris bat coronavirus HKU4, Rousettus bat coronavirus GCCDC1, Rousettus bat coronavirus HKU9, Severe acute respiratory syndrome-related coronavirus.

As used herein, the term “Severe acute respiratory syndrome-related coronavirus”, or SARS virus, includes, in a non-exhaustive manner, the SARS-CoV, SARSr-CoV WIV1, SARSr-CoV HKU3, SARSr-CoV RP3, and SARS-CoV-2; including strains responsible for COVID-19 and their mutants.

As used herein, the term “Deltacoronavirus” refers to the corresponding family of the Baltimore classification, which includes the Andecovirus, Buldecovirus, Herdecovirus, and Moordecovirus subgenus. In a non-exhaustive manner, this includes the following species: Wigeon coronavirus HKU20, Bulbul coronavirus HKU11, Coronavirus HKU15, Munia coronavirus HKU13, White-eye coronavirus HKU16, Night heron coronavirus HKU19, Common moorhen coronavirus HKU21.

As used herein, the term “Gammacoronavirus” refers to the corresponding family of the Baltimore classification, which includes the Cegacovirus and Igacovirus subgenus. In a non-exhaustive manner, this includes the following species: Beluga whale coronavirus SW1 and Avian coronavirus.

According to particular embodiments, the co-crystal of 8-chloro-N-(4-(trifluoromethoxy)phenyl)quinolin-2-amine as defined in the present invention, the pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in a method for treating or preventing a Coronaviridae infection, is for reducing inflammation associated with the Coronaviridae infection.

According to particular embodiments, the co-crystal of 8-chloro-N-(4-(trifluoromethoxy)phenyl)quinolin-2-amine as defined in the present invention, the pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, for use in a method for treating or preventing a Coronaviridae infection, is for reducing the Coronaviridae viral load.

According to particular embodiments, the co-crystal of 8-chloro-N-(4-(trifluoromethoxy)phenyl)quinolin-2-amine as defined in the present invention, the pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention, for use in a method for treating or preventing a Coronaviridae infection, is in combination with:
a dynamin inhibitor, such as Dynasore; and/or
an antibiotic, such as one selected from the group consisting of beta-lactams, fluoroquinolones, and macrolides, such as azythromicin;
remdesivir;
ribavirin;
ritonavir;
lopanivir;
chloroquine or hydroxychloroquine;
beta-interferon;
an anti-inflammatory compound, such as one selected from the group consisting of: anti-TNF, Jak inhibitors, anti-IL6 antibodies, IL6 receptor antagonists; and/or
a calcium inhibitor such as diltiazem.

According to some particular embodiments, the Coronaviridae is selected from Letovirinae and Orthocoronavirinae.

According to some particular embodiments, the Coronaviridae is an Alphacoronavirus or a Betacoronavirus or a Deltacoronavirus or a Gammacoronavirus.

According to some particular embodiments, the Coronaviridae is an Embecovirus or a Hibecovirus or a Merbecobivirus or a Nobecovirus or a Sarbecovirus.

According to some particular embodiments, the Coronaviridae is a Sarbecovirus selected from Severe Acute Respiratory Syndrome-related coronaviruses.

According to some particular embodiments, the Severe Acute Respiratory Syndrome (SARS)-related coronaviruses are selected from the group consisting of: SARS-CoV, SARSr-CoV WIV1, SARSr-CoV HKU3, SARSr-CoV RP3, SARS-CoV-2.

According to some preferred embodiments, the Severe Acute Respiratory Syndrome (SARS)-related coronaviruses are selected from SARS-CoV and SARS-CoV-2; including strains responsible for COVID-19 and their mutants.

According to some embodiments, the co-crystal of 8-chloro-N-(4-(trifluoromethoxy)phenyl)quinolin-2-amine as defined in the present invention, the pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention are used in a method for treating or preventing a Coronaviridae infection, wherein the level of 8-chloro-N-(4-(trifluoromethoxy)phenyl)quinolin-2-amine free base equivalent, in a blood, plasma, tissue, saliva, pharyngeal, tracheal, bronchoalveolar, and/or serum sample of the patient, is measured during the use.

Cancers

A co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention, may be useful in the treatment or prevention of various cancers.

As used herein the term “cancer”, and unless stated otherwise, may relate to any disorder associated with abnormal cell growth, which thus includes malignant tumors and benign tumors, metastatic tumors and non-metastatic tumors, solid tumors and non-solid tumors, such as Blood-Related Cancers which may thus include Leukaemia, Lymphoma and Myeloma; it may also relate to Central Nervous System (CNS) cancers and non-CNS cancers. Unless stated otherwise, the term “cancer” also encompasses juvenile and non-juvenile cancers, Recurrent and Non-Recurrent cancers as well as cancer relapses.

Among cancers, the following may be cited: Blood-Related Cancer, pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, oesophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, oesophageal squamous cell carcinomas, oral carcinomas, cancers of the adrenal cortex, or ACTH-producing tumors.

According to one embodiment, the following cancers may be cited: head and neck cancer, stomach cancer, breast cancer, basal and squamous skin cell cancer, liver cancer, kidney cancer, brain cancer, lung cancer, pancreatic cancer, eye cancer, gastrointestinal cancer, colorectal cancer, oesophageal cancer, colorectal cancer, bladder cancer, gall bladder cancer, thyroid cancer, melanoma, uterine/cervical cancer, ovarian, cancer, bone cancer and renal cancer.

According to another embodiment, the cancer may be selected from head and neck cancer, Head and Neck Squamous Cell Carcinoma, Neck Squamous Cell Carcinoma, Acute Lymphocytic Leukemia (ALL) in Adults or children, Acute Myeloid Leukemia (AML) in adults or children, Acute Lymphoblastic Leukemia, Adrenal Cancer, Anal Cancer, Astrocytic Glioma, Astrocytoma (grade I, II, III, or IV), B- or NK/T-cell lymphomas, Basal and Squamous Skin Cell Cancer, Bile Duct Cancer, Bladder Cancer, Bone Cancer, brain cancer, Brain and Spinal Cord Tumors in Adults, Brain and Spinal Cord Tumors in Children, Anaplastic astrocytomas, Breast cancer, Gastrointestitnal cancer, Breast Cancer in Women, Breast Cancer in Young Women, Breast Cancer in Men, Recurrent Breast Cancer, Hereditary Breast Cancer, HER2 positive Breast Cancer, Breast Cancer associated with lymph node metastatis, ER-alpha positive Breast Cancer, Cancer in Adolescents, Cancer in Children, Cancer in Young Adults, Cancer of Unknown Primary, Castleman Disease, Cervical Cancer, Cervical Intraepithelial Neoplasia, Cholangiocarcinoma, Chronic Lymphocytic Leukemia (CLL), Chronic Myeloid Leukemia (CML), Chronic Myelomonocytic Leukemia (CMML), Colorectal Cancer, colorectal adenoma, Cutaneous Squamous Cell Carcinoma, Endometrial Cancer, Epithelial Ovarian Cancer, Epithelial Ovarian Cancer associated with metastasis, esophageal cancer, esophagus Squamous Cell Carcinoma, Ewing sarcoma, Ewing Family of Tumors, Lymphoblastic leukaemia (ALL), Eye Cancer, such as Ocular Melanoma and Lymphoma, Gallbladder Cancer, Gastric Cancer, Gastrointestinal Carcinoid Tumors, Gastrointestinal Stromal Tumor (GIST), Gestational Trophoblastic Disease, Glioblastoma, Glioblastoma multiforme (GBM), Hairy cell leukemia, Glioma, High-grade glioma, Hepatocellular carcinoma, Intrahepatic cholangiocarcinoma, Invasive Breast Ductal Carcinoma, Hodgkin Lymphoma, Kaposi Sarcoma, Kidney Cancer, Laryngeal and Hypopharyngeal Cancer, Leiomyosarcoma, Leukemia, Leukemia in Children, Liver Cancer, Lung Cancer, Lung Carcinoid Tumor, Lymphoma, Lymphoma of the Skin, Malignant Mesothelioma, Mantle cell lymphoma, Medulloblastoma, Melanoma Skin Cancer, malignant melanoma, Meningioma, Merkel Cell Skin Cancer, Multiple Myeloma, Multiple Myeloma with Osteonecrosis of the Jaw, Myelodysplastic Syndrome, Nasal Cavity and Paranasal Sinuses Cancer, Nasopharyngeal Cancer, recurrent or metastatic Nasopharyngeal carcinoma, Neuroblastoma, Neuroglioma, Non-Hodgkin Lymphoma, Non-Hodgkin Lymphoma in Children, Non-Small Cell Lung Cancer, Gefitinib-resistant non-small cell lung cancer, Oral cancer, Oral Cavity and Oropharyngeal Cancer, Osteosarcoma, Pulmonary Metastatic Osteosarcoma, Ovarian Cancer, Pancreatic Cancer, thyroid carcinoma, Papillary Thyroid Carcinoma, Pediatric Spinal Ependymoma, Penile Cancer, Pituitary Tumors, Pituitary Adenoma, Proneural tumors, Prostate Cancer, Retinoblastoma, Rhabdomyosarcoma, Salivary Gland Cancer, Skin Cancer, Small Cell Lung Cancer, Small Intestine Cancer, Soft Tissue Sarcoma, Squamous Cell Carcinoma of the Tongue, Stomach Cancer, Testicular Cancer, Thymus Cancer, Thyroid Cancer, Uterine Sarcoma, Vaginal Cancer, Vulvar Cancer, Renal cancer, Retinoblastoma, Waldenstrom Macroglobulinemia and Wilms Tumor.

According to another embodiment, the cancer may be selected from a Head and Neck Squamous Cell Carcinoma, Neck Squamous Cell Carcinoma, Acute Lymphocytic Leukemia (ALL) in Adults or children, Acute Myeloid Leukemia (AML) in adults or children, Acute Lymphoblastic Leukemia, Adrenal Cancer, Anal Cancer, Astrocytic Glioma, Astrocytoma (grade I, II, III, or IV), B- or NK/T-cell lymphomas, Basal and Squamous Skin Cell Cancer, Bile Duct Cancer, Bone Cancer, brain cancer, Brain and Spinal Cord Tumors in Adults, Brain and Spinal Cord Tumors in Children, Anaplastic astrocytomas, Gastrointestitnal cancer, Breast Cancer in Women, Breast Cancer in Young Women, Breast Cancer in Men, Recurrent Breast Cancer, Hereditary Breast Cancer, HER2 positive Breast Cancer, Breast Cancer associated with lymph node metastatis, ER-alpha positive Breast Cancer, Cancer in Adolescents, Cancer in Children, Cancer in Young Adults, Cancer of Unknown Primary, Castleman Disease, Cervical Intraepithelial Neoplasia, Cholangiocarcinoma, Chronic Lymphocytic Leukemia (CLL), Chronic Myeloid Leukemia (CML), Chronic Myelomonocytic Leukemia (CMML), colorectal adenoma, Cutaneous Squamous Cell Carcinoma, Endometrial Cancer, Epithelial Ovarian Cancer, Epithelial Ovarian Cancer associated with metastasis, esophagus Squamous Cell Carcinoma, Ewing sarcoma, Ewing Family of Tumors, Lymphoblastic leukaemia (ALL), Eye Cancer, such as Ocular Melanoma and Lymphoma, Gastric Cancer, Gastrointestinal Carcinoid Tumors, Gastrointestinal Stromal Tumor (GIST), Gestational Trophoblastic Disease, Glioblastoma, Glioblastoma multiforme (GBM), Hairy cell leukemia, Glioma, High-grade glioma, Hepatocellular carcinoma, Intrahepatic cholangiocarcinoma, Invasive Breast Ductal Carcinoma, Hodgkin Lymphoma, Kaposi Sarcoma, Laryngeal and Hypopharyngeal Cancer, Leiomyosarcoma, Leukemia, Leukemia in Children, Lung Carcinoid Tumor, Lymphoma, Lymphoma of the Skin, Malignant Mesothelioma, Mantle cell lymphoma, Medulloblastoma, malignant melanoma, Meningioma, Merkel Cell Skin Cancer, Multiple Myeloma, Multiple Myeloma with Osteonecrosis of the Jaw, Myelodysplastic Syndrome, Nasal Cavity and Paranasal Sinuses Cancer, Nasopharyngeal Cancer, recurrent or metastatic Nasopharyngeal carcinoma, Neuroblastoma, Neuroglioma, Non-Hodgkin Lymphoma, Non-Hodgkin Lymphoma in Children, Gefitinib-resistant non-small cell lung cancer, Oral cancer, Oral Cavity and Oropharyngeal Cancer, Osteosarcoma, Pulmonary Metastatic Osteosarcoma, thyroid carcinoma, Papillary Thyroid Carcinoma, Pediatric Spinal Ependymoma, Penile Cancer, Pituitary Tumors, Pituitary Adenoma, Proneural tumors, Retinoblastoma, Rhabdomyosarcoma, Salivary Gland Cancer, Skin Cancer, Small Cell Lung Cancer, Small Intestine Cancer, Soft Tissue Sarcoma, Squamous Cell Carcinoma of the Tongue, Testicular Cancer, Thymus Cancer, Uterine Sarcoma, Vaginal Cancer, Vulvar Cancer, Renal cancer, Retinoblastoma, Waldenstrom Macroglobulinemia and Wilms Tumor.

According to a further embodiment, the cancer may be selected from head and neck cancer, Head and Neck Squamous Cell Carcinoma, Neck Squamous Cell Carcinoma, Malignant melanoma, stomach cancer, Breast cancer, Breast cancer in Women, Breast Cancer in Young Women, basal and squamous skin cell cancer, liver cancer, brain cancer, Anaplastic astrocytomas, lung cancer, Non-Small Cell Lung Cancer, Gefitinib-resistant non-small cell lung cancer, Oral cancer, eye cancer, Gastric Cancer, gastrointestinal cancer, Astrocytic Glioma, Astrocytoma (grade I, II, III, or IV), colorectal cancer, colorectal adenoma, Cutaneous Squamous Cell Carcinoma, bladder cancer, bone cancer, Recurrent Breast Cancer, Hereditary Breast Cancer, HER2 positive Breast Cancer, Breast Cancer associated with lymph node metastatis, ER-alpha positive Breast Cancer, renal cancer, Cervical Intraepithelial Neoplasia, Cholangiocarcinoma, Leiomyosarcoma, Chronic Lymphocytic Leukemia (CLL), Chronic Myeloid Leukemia (CML), Chronic Myelomonocytic Leukemia (CMML), Acute Myeloid Leukemia (AML) in adults or children, Acute Lymphoblastic Leukemia, B- or NK/T-cell lymphomas, cervical cancer, Glioblastoma, Glioblastoma multiforme (GBM), Hairy cell leukemia, Glioma, High-grade glioma, Hepatocellular carcinoma, Intrahepatic cholangiocarcinoma, Invasive Breast Ductal Carcinoma, kidney cancer, Endometrial cancer, ovarian cancer, Epithelial Ovarian Cancer, Epithelial Ovarian Cancer associated with metastasis, esophageal cancer, esophageal Squamous Cell Carcinoma, Ewing sarcoma, Lymphoblastic leukaemia (ALL), Mantle cell lymphoma, Medulloblastoma, Lymphoma, Myelodysplastic syndrome, Meningioma, Multiple Myeloma (MM), Multiple Myeloma with Osteonecrosis of the Jaw, Nasopharyngeal Cancer, recurrent or metastatic Nasopharyngeal carcinoma, Neuroblastoma, Neuroglioma, Papillary Thyroid Carcinoma, Pediatric Spinal Ependymoma, Osteosarcoma, Pulmonary Metastatic Osteosarcoma, pancreatic cancer, thyroid carcinoma, sarcoma, pituitary tumors, Pituitary Adenoma, Proneural tumors, Squamous Cell Carcinoma of the Tongue, Mesothelioma, Retinoblastoma and prostate cancer.

According to a further embodiment, the cancer may be selected from Head and Neck cancer, Head and Neck Squamous Cell Carcinoma, Neck Squamous Cell Carcinoma, malignant melanoma, Astrocytic Glioma, Glioma, stomach cancer, Breast cancer, Cholangiocarcinoma, recurrent or metastatic Nasopharyngeal carcinoma, basal and squamous skin cell cancer, liver cancer, brain cancer, Anaplastic astrocytomas, lung cancer, Non-Small Cell Lung Cancer, Gefitinib-resistant non-small cell lung cancer, Oral cancer, Glioblastoma, osteosarcoma, Pulmonary Metastatic Osteosarcoma, pancreatic cancer, eye cancer, gastrointestinal cancer, colorectal cancer, colorectal adenoma, Cutaneous Squamous Cell Carcinoma, Endometrial cancer, Epithelial Ovarian Cancer, esophageal cancer, Ewing sarcoma, gastric cancer, Hepatocellular carcinoma, HER2 positive Breast Cancer, bladder cancer, bone cancer, prostate cancer, Retinoblastoma and renal cancer.

According to a further embodiment, the cancer may be selected from Anaplastic astrocytomas, Astrocytic gliomas, Bladder cancer, Breast cancer, Cholangiocarcinoma, Colorectal cancer, Colorectal adenoma, Cutaneous squamous cell carcinoma, Endometrial cancer, Epithelial ovarian cancer, Esophageal cancer, Ewing sarcoma, Gastric cancer, Gefitinib-resistant non-small cell lung cancer, Glioblastoma, Glioma, Hepatocellular carcinoma, HER2 positive breast cancer, Head and Neck Squamous Cell Carcinoma, Malignant melanoma, Nasopharyngeal carcinoma (recurrence or metastasis), Neck squamous cell carcinoma, Non-small cell lung cancer, Oral cancer, Osteosarcoma, Osteosarcoma (pulmonary metastasis), Prostate cancer and retinoblastoma

According to a further embodiment, the cancer may be selected from anal cancer, bile duct cancer, gastrointestinal cancer, Cholangiocarcinoma, colorectal cancer, colorectal adenoma, esophageal cancer, Esophagus Squamous Cell Carcinoma, gastric cancer, Gastrointestinal Carcinoid Tumors, Gastrointestinal Stromal Tumor (GIST), Hepatocellular carcinoma, Intrahepatic cholangiocarcinoma, liver cancer, lung cancer, Lung Carcinoid Tumor, non-Small Cell Lung Cancer, Gefitinib-resistant non-small cell lung cancer, Pulmonary Metastatic Osteosarcoma, stomach cancer, pancreatic cancer, Small Cell Lung Cancer, and Small Intestine Cancer

According to one embodiment, the patient does not present clinically detectable metastases, in particular said patient has a pre-cancerous condition, an early stage cancer or a non-metastatic cancer, or said patient presents clinically detectable metastases and said co-crystal of 8-chloro-N-(4-(trifluoromethoxy)phenyl)quinolin-2-amine as defined in the present invention, or said pharmaceutically acceptable salt of 8-chloro-N-(4-(trifluoromethoxy)phenyl)quinolin-2-amine including a solvate and/or a hydrate thereof as defined in the present invention does not target directly the invasion of metastases.

In view of the above, the invention relates to a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use in the treatment and/or prevention of cancer, such as the here above listed cancers, and dysplasia.

Thus, the invention also relates to the use of a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention for treating and/or preventing cancer, such as the here above listed cancers, and dysplasia.

The invention also relates to the use of a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention for the preparation of a composition, such as a medicament, for treating and/or preventing of cancer, such as the here above listed cancers, and dysplasia.

The invention also relates to a method of preventing, inhibiting or treating cancer or dysplasia, which comprises at least one step consisting in administering to a patient suffering therefrom an effective amount of a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention.

In some embodiments, the invention relates to a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating and/or preventing cancer or dysplasia, wherein a presence and/or expression level of miR-124 in a blood and/or tissue sample of the patient, is measured prior to and/or during the use.

In some embodiments, the invention relates to a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating and/or preventing cancer or dysplasia, wherein a presence and/or expression level of miR-124 in a blood and/or tissue sample is measured to guide dose or monitor response to the treatment.

In some embodiments, the invention relates to a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating and/or preventing cancer or dysplasia, wherein the level of 8-chloro-N-(4-(trifluoromethoxy)phenyl)quinolin-2-amine free base equivalent, in a blood, plasma, tissue, saliva, and/or serum sample of the patient is measured during the use.

In some embodiments, the invention relates to a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating and/or preventing cancer or dysplasia, which is used in combination with another anti-tumoral agent.

In some embodiments, the invention relates to a method of the present invention or a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin-2-amine including a solvate and/or a hydrate thereof as defined in the present invention, or a pharmaceutical composition as defined in the present invention for use as defined above, for treating and/or preventing cancer or dysplasia, which is used in combination with another therapy selected from chemotherapy, immunotherapy, radiotherapy, surgery, ultrasounds, monoclonal antibodies, and cancer vaccines.

Among other anticancer drug, the following may be cited:
Androgen receptor inhibitors, such as enzalutamide (Xtandi®, Astellas/Medivation), abiraterone (Zytiga®, Centocor/Ortho), antagonist of gonadotropin-releasing hormone (GnRH) receptor such as degaralix, Firmagon®, Ferring Pharmaceuticals)
Antiapoptotics, such as venetoclax (Venclexta®, AbbVie/Genentech), blinatumomab (Blincyto®, Amgen), navitoclax (ABT-263, Abbott);
Antiproliferative and Antimitotic agents, such as vinca alkaloids (which include vinblastine, vincristine);
Antibiotics such as dactinomycin, daunorubicin, doxorubicin, idarubicin, anthracyclines, mitoxantrone, bleomycins, plicamycin (mithramycin), and mitomycin;
L-asparaginase;
Antiplatelet agents;
Antiproliferative/antimitotic alkylating agents such as nitrogen mustards cyclophosphamide and analogs (which include melphalan, chlorambucil, hexamethylmelamine, and thiotepa), alkyl nitrosoureas (which include carmustine) and analogs, streptozocin, and triazenes (which include dacarbazine);
Antiproliferative/antimitotic antimetabolites such as folic acid analogs (which include methotrexate), aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd6244 from AstraZeneca, PD181461 from Pfizer and leucovorin;
Antimigratory agents;
Angiogenesis inhibitors, such as TNP-470;
Aromatase inhibitors, such as letrozole and anastrozole, exemestane;
Angiotensin
Anti-sense oligonucleotides, such as antisense nucleic acids directed toward miR-124;
Anticoagulants, such as heparin, synthetic heparin salts, and other inhibitors of thrombin;
Arginine inhibitors, such as AEB 1102 (pegylated recombinant arginase, Aeglea Biotherapeutics) and CB-1158 (Calithera Biosciences);
Bone resorption inhibitors, such as Denosumab (Xgeva®, Amgen), bisphosphonates such as zoledronic acid (Zometa®, Novartis);
CC chemokine receptor 4 (CCR4) inhibitors, such as mogamulizumab (Poteligeo®, Kyowa Hakko Kirin, Japan);
CDK inhibitors, such as CDK4/CDK6 inhibitors, such as palbociclib (Ibrance®, Pfizer); ribociclib (Kisqali®, Novartis); abemaciclib (Ly2835219, Eli Lilly); and trilaciclib (G1T28, G1 Therapeutics);
Cell cycle inhibitors and differentiation inducers, such as as tretinoin;
Corticosteroids, such as cortisone, dexamethasone, hydrocortisone, methylprednisolone, prednisone, and prednisolone;
DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide;
Fibrinolytic agents, such as tissue plasminogen activator, streptokinase, urokinase, aspirin, dipyridamole, ticlopidine, and clopidogrel;
Folate antagonists;
FLT3 receptor inhibitors, such as enzalutamide, abiraterone, apalutamide, erlotinib, crizotinib, niraparib, olaparib, osimertinib, regorafenib, sunitinib, lestaurtinib, midostaurin, gilteritinib, semaxinib, linifanib, fostamatinib, pexidartinib, sorafenib, cabozantinib, ponatinib, ilorasertib, pacritinib, famitinib, pexidartinib, quizartinib;
Glutaminase inhibitors, such as CD-839 (Calithera Biosciences);
Growth Factor Signal transduction kinase inhibitors;
Growth factor Inhibitors, such as vascular endothelial growth factor inhibitors and fibroblast growth factor inhibitors, such as olaratumab (Lartruvo®; Eli Lilly), cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca);
Hedgehog pathway inhibitors, such as sonidegib (Odomzo®, Sun Pharmaceuticals); and vismodegib (Erivedge®, Genentech);
Histone deacetylase (HDAC) inhibitors, such as vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China);
Hormones and analogs thereof, such as estrogen, tamoxifen, goserelin, bicalutamide, and nilutamide);
Isocitrate dehydrogenase (IDH) inhibitors, such as AG120 (Celgene; NCT02677922); AG221 (Celgene, NCT02677922; NCT02577406); BAY1436032 (Bayer, NCT02746081); IDH305 (Novartis, NCT02987010)
Isoflavones such as genistein;
Immunosuppressives, such as tacrolimus, sirolimus, azathioprine, and mycophenolate;
Inhibitors of p53 suppressor proteins, such as ALRN-6924 (Aileron);
Inhibitors of transforming growth factor-beta (TGF-beta or TGFß), such as NIS793 (Novartis), fresolimumab (GC1008; Sanofi-Genzyme), M7824 (Merck KgaA—formerly MSB0011459X);
iNKT cell agonists asuch as ABX196 5Abivax)
mTOR inhibitors, such as everolimus (Afinitor®, Novartis); temsirolimus (Torisel®, Pfizer); and sirolimus (Rapamune®, Pfizer);
Microtubule-inhibiting drugs, such as taxanes (which include paclitaxel, docetaxel), vinblastin, nocodazole, epothilones, vinorelbine) (NAVELBINE®), and epipodophyllotoxins (etoposide, teniposide);
Nitric oxide donors;
Nucleoside inhibitors, such as trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology);
PI3K inhibitors, such as idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics);
Platinum coordination complexes (such as cisplatin, oxiloplatin, carboplatin, nedaplatin, picoplatin, procarbazine, mitotane, satraplatin and aminoglutethimide;
Poly ADB ribose polymerase (PARP) inhibitor, such as those selected from: olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.);
Proteasome inhibitors, such as everolimus (Afinitor®, Novartis); temsirolimus (Torisel®, Pfizer); and sirolimus (Rapamune®, Pfizer), bortezomib (Velcade®, Takeda); carfilzomib (Kyprolis®, Amgen); and ixazomib (Ninlaro®, Takeda);
Pyrimidine & Purine analogs, such as floxuridine, capecitabine, and cytarabine;
Receptor blockers, Antisecretory agents, such as breveldin;
Selective estrogen receptor modulator (SERM), such as raloxifene (Evista®, Eli Lilly);
Therapeutic antibodies, such as those selected from: anti-TNF antibodies, anti-VEGF antibodies, anti-EGFR antibodies, anti-PD-1 antibodies, anti-HER2 antibodies, anti-CD20 antibodies, anti-IL17 antibodies, and anti-CTLA4 antibodies, anti-PDL1, anti-CD25, anti-α4integrin, anti-IL6R, anti-05, anti-IL1, anti-TPO, anti-IL12/23, anti-EPCAM/CD3, anti-CD30, anti-CD80/86, anti-anthrax, anti-CCR4, anti-CD6, anti-CD19, anti-α4β7, anti-IL6, anti-VEGFR-2, anti-SLAMF7, anti-GD2, anti-IL17A, anti-PCSK9, anti-IL5, anti-CD22, anti-IL4, anti-PDGFRα, anti-IL17RA and anti-TcdB, and such as those selected from: Abagovomab, Abatacept, Abciximab, Abituzumab, Abrilumab, Actoxumab, Adalimumab, Adecatumab, Aducanumab, Aflibercept, Afutuzymab, Alacizumab, Alefacept, Alemtuzumab, Alirocumab, Altumomab, Amatixumab, Anatumomab, Anetumab, Anifromumab, Anrukinzumab, Apolizumab, Arcitumomab, Ascrinvacumab, Aselizumab, Atezolizumab, Atinumab, Altizumab, Atorolimumab, Bapineuzumab, Basiliximab, Bavituximab, Bectumomab, Begelomab, Belatacept, Belimumab, Benralizumab, Bertilimumab, Besilesomab, Bevacizumab, Bezlotoxumab, Biciromab, Bimagrumab, Bimekizumab, Bivatuzumab, Blinatumomab, Blosozumab, Bococizumab, Brentuximab, Briakimumab, Brodalumab, Brolucizumab, Bronticizumab, Canakinumab, Cantuzumab, Caplacizumab, Capromab, Carlumab, Catumaxomab, Cedelizumab, Certolizumab, Cetixumab, Citatuzumab, Cixutumumab, Clazakizumab, Clenoliximab, Clivatuzumab, Codrituzumab, Coltuximab, Conatumumab, Concizumab, Crenezumab, Dacetuzumab, Daclizumab, Dalotuzumab, Dapirolizumab, Daratumumab, Dectrekumab, Demcizumab, Denintuzumab, Denosumab, Derlotixumab, Detumomab, Dinutuximab, Diridavumab, Dorlinomab, Drozitumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab, Enlimomab, Enoblituzumab, Enokizumab, Enoticumab, Ensituximab, Epitumomab, Epratuzomab, Erlizumab, Ertumaxomab, Etanercept, Etaracizumab, Etrolizumab, Evinacumab, Evolocumab, Exbivirumab, Fanolesomab, Faralimomab, Farletuzomab, Fasimumab, Felvizumab, Fezkimumab, Ficlatuzumab, Figitumumab, Firivumab, Flanvotumab, Fletikumab, Fontolizumab, Foralumab, Foravirumab, Fresolimumab, Fulramumab, Futuximab, Galiximab, Ganitumab, Gantenerumab, Gavilimomab, Gemtuzumab, Gevokizumab, Girentuximab, Glembatumumab, Golimumab, Gomiliximab, Guselkumab, Ibalizumab, Ibritumomab, Icrucumab, Idarucizumab, Igovomab, Imalumab, Imciromab, Imgatuzumab, Inclacumab, Indatuximab, Indusatumab, Infliximab, Intetumumab, Inolimomab, Inotuzumab, Ipilimumab, Iratumumab, Isatuximab, Itolizumab, Ixekizumab, Keliximab, Labetuzumab, Lambrolizumab, Lampalizumab, Lebrikizumab, Lemalesomab, Lenzilumab, Lerdelimumab, Lexatumumab, Libivirumab, Lifastuzumab, Ligelizumab, Lilotomab, Lintuzumab, Lirilumab, Lodelcizumab, Lokivetmab, Lorvotuzumab, Lucatumumab, Lulizumab, Lumiliximab, Lumretuzumab, Mapatumumab, Margetuximab, Maslimomab, Mavrilimumab, Matuzumab, Mepolizumab, Metelimumab, Milatuzumab, Minetumomab, Mirvetuximab, Mitumomab, Mogamulizumab, Morolimumab, Motavizumab, Moxetumomab, Muromonab-CD3, Nacolomab, Namilumab, Naptumomab, Narnatumab, Natalizumab, Nebacumab, Necitumumab, Nemolizumab, Nerelimomab, Nesvacumab, Nimotuzumab, Nivolumab, Nofetumomab, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab, Oregovomab, Orticumab, Otelixizumab, Oltertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab, Pintumomab, Polatuzumab, Ponezumab, Priliximab, Pritumumab, Quilizumab, Racotumomab, Radretumab, Rafivirumab, Ralpancizumab, Ramucirumab, Ranibizumab, Raxibacumab, Refanezumab, Regavirumab, Reslizumab, Rilonacept, Rilotumumab, Rinucumab, Rituximab, Robatumumab, Roledumab, Romosozumab, Rontalizumab, Rovelizumab, Ruplizumab, Sacituzumab, Samalizumab, Sarilumab, Satumomab, Secukimumab, Seribantumab, Setoxaximab, Sevirumab, Sibrotuzumab, Sifalimumab, Siltuximab, Siplizumab, Sirukumab, Sofituzumab, Solanezumab, Solitomab, Sonepcizumab, Sontuzumab, Stamulumab, Sulesomab, Suvizumab, Tabalumab, Tacatuzumab, Tadocizumab, Talizumab, Tanezumab, Taplitumomab, Tarextumab, Tefibazumab, Telimomab aritox, Tenatumomab, Teneliximab, Teplizumab, Tesidolumab, TGN 1412, Ticlimumab, Tildrakizumab, Tigatuzumab, TNX-650, Tocilizumab, Toralizumab, Tosatoxumab, Tositumomab, Tovetumab, Tralokimumab, Trastuzumab, TRBS07, Tregalizumab, Tremelimumab, Trevogrumab, Tucotuzumab, Tuvirumab, Ublituximab, Ulocuplumab, Urelumab, Urtoxazumab, Ustekimumab, Vandortuzumab, Vantictumab, Vanucizumab, Vapaliximab, Varlimumab, Vatelizumab, Vedolizumab, Veltuzumab, Vepalimomab, Vesencumab, Visilizumab, Volocixumab, Vorsetuzumab, Votumumab, Zalutumimab, Zanolimumab, Zatuximab, Ziralimumab, Ziv-Aflibercept, and Zolimomab;
Topoisomerase inhibitors, such as doxorubicin, daunorubicin, dactinomycin, eniposide, epirubicin, etoposide, idarubicin, irinotecan, mitoxantrone, topotecan, and irinotecan;
Toxins, such as Cholera toxin, ricin, Pseudomonas exotoxin, Bordetella pertussis adenylate cyclase toxin, diphtheria toxin, and caspase activators;

Kinase or VEGF inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Tressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis), tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda);

In a non-limitative manner, co-crystals or salts of the invention may be combined, alone or in the form of a kit-of-parts, to one or more of the following anti-cancer drugs or compounds: ABVD, AC, ACE, Abiraterone (Zytiga®), Abraxane, Abstral, Actinomycin D, Actiq, Adriamycin, Afatinib (Giotrif®), Afinitor, Aflibercept (Zaltrap®), Aldara, Aldesleukin (IL-2, Proleukin or interleukin 2), Alemtuzumab (MabCampath), Alkeran, Amsacrine (Amsidine, m-AMSA), Amsidine, Anastrozole (Arimidex®), Ara C, Aredia, Arimidex, Aromasin, Arsenic trioxide (Trisenox®, ATO), Asparaginase (Crisantaspase®, Erwinase®), Axitinib (Inlyte®), Azacitidine (Vidaze®), BEACOPP, BEAM, Bendamustine (Levact®), Bevacizumab (Avastin), Bexarotene (Targretin®), Bicalutamide (Casodex®), Bleomycin, Bleomycin, etoposide and platinum (BEP), Bortezomib (Velcade®), Bosulif, Bosutinib (Bosulif), Brentuximab (Adcetris®), Brufen, Buserelin (Suprefact®), Busilvex, Busulfan (Myleran, Busilvex), CAPE-OX, CAPDX, CAV, CAVE, CCNU, CHOP, CMF, CMV, CVP, Cabazitaxel (Jevtana®), Cabozantinib (Cometriq®), Caelyx, Calpol, Campto, Capecitabine (Xeloda®), Caprelsa, Carbo MV, CarboTaxol, Carboplatin, Carboplatin and etoposide, Carboplatin and paclitaxel, Carmustine (BCNU, Gliadel®), Casodex, Ceritinib (Zykadia®), Cerubidin, Cetuximab (Erbitux®), ChlVPP, Chlorambucil (Leukeran®), Cisplatin, Cisplatin and Teysuno, Cisplatin and capecitabine (CX), Cisplatin, etoposide and ifosfamide (PEI), Cisplatin, fluorouracil (5-FU) and trastuzumab, Cladribine (Leustat®, LITAK), Clasteon, Clofarabine (Evoltra®), Co-codamol (Kapake®, Solpadol®, Tylex®), Cometriq, Cosmegen, Crisantaspase, Crizotinib (Xalkori®), Cyclophosphamide, Cyclophosphamide, thalidomide and dexamethasone (CTD), Cyprostat, Cyproterone acetate (Cyprostat®), Cytarabine (Ara C, cytosine arabinoside), Cytarabine into spinal fluid, Cytosine arabinoside, DHAP, DTIC, Dabrafenib (Tafinlar®), Dacarbazine (DTIC), Dacogen, Dactinomycin (actinomycin D, Cosmegen®), Dasatinib (Sprycel), Daunorubicin, De Gramont, Decapeptyl SR, Decitabine (Dacogen®), Degarelix (Firmagon®), Denosumab (Praia®, Xgeva®), Depocyte, Dexamethasone, Diamorphine, Disodium pamidronate, Disprol, Docetaxel (Taxotere®), Docetaxel, cisplatin and fluorouracil (TPF), Doxifos, Doxil, Doxorubicin (Adriamycin), Doxorubicin and ifosfamide (Doxifos), Drogenil, Durogesic, EC, ECF, EOF, EOX, EP, ESHAP, Effentora, Efudix, Eldisine, Eloxatin, Enzalutamide, Epirubicin (Pharmorubicin®), Epirubicin cisplatin and capecitabine (ECX), Epirubicin, carboplatin and capecitabine (ECarboX), Eposin, Erbitux, Eribulin (Halaven®), Erlotinib (Tarceva®), Erwinase, Estracyt, Etopophos, Etoposide (Eposin®, Etopophos®, Vepesid®), Everolimus (Afinitor®), Evoltra, Exemestane (Aromasin®), FAD, FEC, FEC-T chemotherapy, FMD, FOLFIRINOX, FOLFOX, Faslodex, Femara, Fentanyl, Firmagon, Fludara, Fludarabine (Fludara®), Fludarabine, cyclophosphamide and rituximab (FCR), Fluorouracil (5FU), Flutamide, Folinic acid, fluorouracil and irinotecan (FOLFIRI), Fulvestrant (Faslodex®), G-CSF, Gefitinib (Iressa), GemCarbo (gemcitabine and carboplatin), GemTaxol, Gemcitabine (Gemzar), Gemcitabine and capecitabine (GemCap), Gemcitabine and cisplatin (GC), Gemcitabine and paclitaxel (GemTaxol®), Gemzar, Giotrif, Gliadel, Glivec, Gonapeptyl, Depot, Goserelin (Zoladex®), Goserelin (Zoladex®, Novgos®), Granulocyte colony stimulating factor (G-CSF), Halaven, Herceptin, Hycamtin, Hydrea, Hydroxycarbamide (Hydrea®), Hydroxyurea, I-DEX, ICE, IL-2, IPE, Ibandronic acid, Ibritumomab (Zevalin®), Ibrutinib (Imbruvica®), Ibuprofen (Brufen®, Nurofen®), Iclusig, Idarubicin (Zavedos®), Idarubicin and dexamethasone, Idelalisib (Zydelig®), Ifosfamide (Mitoxana®), Imatinib (Glivec®), Imiquimod cream (Aldara®), Imnovid, Instanyl, Interferon (Intron A), Interleukin, Intron A, Ipilimumab (Yervoy®), Iressa, Ifinotecan (Campto®), Ifinotecan and capecitabine (Xeliri®), Ifinotecan de Gramont, Ifinotecan modified de Gramont, Javlor, Jevtana, Kadcyla, Kapake, Keytruda, Lanreotide (Somatuline®), Lanvis, Lapatinib (Tyverb®), Lenalidomide (Revlimid®), Letrozole (Femara®), Leukeran, Leuprorelin (Prostap®, Lutrate®), Leustat, Levact, Liposomal doxorubicin, Litak, Lomustine (CCNU), Lynparza, Lysodren, MIC, MMM, MPT, MST Continus, MVAC, MVP, MabCampath, Mabthera, Maxtrex, Medroxyprogesterone acetate (Provera), Megace, Megestrol acetate (Megace®), Melphalan (Alkeran®), Mepact, Mercaptopurine (Xaluprine®), Methotrexate (Maxtrex), Methyl prednisolone, Mifamurtide (Mepact®), Mitomycin C, Mitotane, Mitoxana, Mitoxantrone (Mitozantrone®), Morphgesic SR, Morphine, Myleran, Myocet, Nab-paclitaxel, Nab-paclitaxel (Abraxane®), Navelbine, Nelarabine (Affiance), Nexavar, Nilotinib (Tasigna®), Nintedanib (Vargatef®), Nipent, Nivolumab (Opdivo®), Novgos, Nurofen, Obinutuzumab (Gazyvaro®), Octreotide, Ofatumumab (Arzerra®), Olaparib (Lynparza®), Oncovin, Onkotrone, Opdivo, Oramorph, Oxaliplatin (Eloxatin), Oxaliplatin and capecitabine (Xelox®), PAD, PC (paclitaxel and carboplatin, CarboTaxol), PCV, PE, PMitCEBO, POMB/ACE, Paclitaxel (Taxol®), Paclitaxel and carboplatin, Pamidronate, Panadol, Panitumumab (Vectibix®), Paracetamol, Pazopanib (Votrient®), Pembrolizumab (Keytruda), Pemetrexed (Alimta®), Pemetrexed and carboplatin, Pemetrexed and cisplatin, Pentostatin (Nipent®), Perjeta, Pertuzumab (Perjeta®), Pixantrone (Pixuvri®), Pixuvri, Pomalidomide (Imnovid®), Ponatinib, Potactasol, Prednisolone, Procarbazine, Proleukin, Prolia, Prostap, Provera, Purinethol, R-CHOP, R-CVP, R-DHAP, R-ESHAP, R-GCVP, RICE, Raloxifene, Raltitrexed (Tomudex®), Regorafenib (Stivarga®), Revlimid, Rituximab, (Mabthera®), Sevredol, Sodium clodronate (Bonefos®, Clasteon®, Loron®), Solpadol, Sorafenib (Nexavar®), Steroids (dexamethasone, prednisolone, methylprednisolone), Streptozocin (Zanosar®), Sunitinib (Sutent®), Sutent, TAC, TIP, Tafinlar, Tamoxifen, Tarceva, Targretin, Tasigna, Taxol, Taxotere, Taxotere and cyclophosphamide (TC), Temodal, Temozolomide, (Temodal®), Temsirolimus (Torisel®), Tepadina, Teysuno, Thalidomide, Thiotepa (Tepadina®), Tioguanine (Thioguanine®, 6-TG, 6-tioguanine), Tomudex, Topotecan (Hycamtin, Potactasol), Torisel, Trabectedin (Yondelis), Trastuzumab (Herceptin®), Trastuzumab emtansine (Kadcyla®), Treosulfan, Tretinoin (Vesanoid®, ATRA), Triptorelin (Decapeptyl SR®, Gonapeptyl Depot®), Trisenox, Tylex, Tyverb, VIDE, Vandetanib (Caprelsa®), Vargatef, VeIP, Vectibix, Velbe, Velcade, Vemurafenib (Zelboraf®), Vepesid, Vesanoid, Vidaza, Vinblastine (Velbe®), Vincristine, Vincristine, actinomycin D (Dactinomycin®) and cyclophosphamide (VAC), Vincristine, actinomycin and ifosfamide (VAI), Vincristine, doxorubicin and dexamethasone (VAD), Vindesine (Eldisine®), Vinflunine (Javlor®), Vinorelbine (Navelbine), Vismodegib (Erivedge®), Votrient, XELOX, Xalkori, Xeloda, Xgeva, Xtandi, Yervoy, Yondelis, Z-DEX, Zaltrap, Zanosar, Zavedos, Zelboraf, Zevalin, Zoladex (breast cancer), Zoladex (prostate cancer), Zoledronic acid (Zometa®), Zometa, Zomorph, Zydelig, Zytiga.

According to a particular embodiment, a co-crystal of 8-chloro-N-(4-(trifluoromethoxy)phenyl)quinolin-2-amine as defined in the present invention, or a pharmaceutically acceptable salt of 8-chloro-N-(4-(trifluoromethoxy)phenyl)quinolin-2-amine including a solvate and/or a hydrate thereof as defined in the present invention, as described herein, can be combined with various chemotherapies, immunotherapy (e.g. check-point inhibitors, monoclonal antibodies), anti-tumoral vaccines, RNA vaccines, magnetic particles, intravascular microrobots, radiotherapy, surgery, ultrasounds or other anti-tumoral therapies.

Therefore, the present invention further provides a co-crystal of 8-chloro-N-(4-(trifluoromethoxy)phenyl)quinolin-2-amine as defined in the present invention, a pharmaceutically acceptable salt of 8-chloro-N-(4-(trifluoromethoxy)phenyl)quinolin amine including a solvate and/or a hydrate thereof as defined in the present invention, or a pharmaceutical composition as defined in the present invention for use as an antitumor agent intended for patients who are also treated with anyone of immunotherapy, anti-tumoral vaccines, RNA vaccines, radiotherapy, surgery, ultrasounds or other anti-tumoral therapies.

According to one embodiment, the present invention relates to the co-crystals of 8-chloro-N-(4-(trifluoromethoxy)phenyl)quinolin-2-amine for use as defined above, the pharmaceutically acceptable salts of 8-chloro-N-(4-(trifluoromethoxy)phenyl)quinolin-2-amine for use as defined above, or the pharmaceutical composition for use as defined above, wherein the level of 8-chloro-N-(4-(trifluoromethoxy)phenyl)quinolin-2-amine free base equivalent, in a blood, plasma, tissue, saliva, and/or serum sample of the patient is measured during the use.

According to another embodiment, the present invention also relates to the co-crystals of 8-chloro-N-(4-(trifluoromethoxy)phenyl)quinolin-2-amine for use as defined above, the pharmaceutically acceptable salts of 8-chloro-N-(4-(trifluoromethoxy)phenyl)quinolin-2-amine for use as defined above, or the pharmaceutical composition for use as defined above, wherein the use is intended for a patient whose level of 8-chloro-N-(4-(trifluoromethoxy)phenyl)quinolin-2-amine free base equivalent, in a blood, plasma, tissue, saliva, and/or serum sample of the patient is measured during the use.

According to still another embodiment, the present invention also relates to the co-crystals of 8-chloro-N-(4-(trifluoromethoxy)phenyl)quinolin-2-amine for use as defined above, the pharmaceutically acceptable salts of 8-chloro-N-(4-(trifluoromethoxy)phenyl)quinolin-2-amine for use as defined above, or the pharmaceutical composition for use as defined above, wherein a presence and/or expression level of miR-124 in a blood and/or tissue sample of the patient, is measured prior to and/or during the use, in particular for monitoring efficacy of the use and/or response to the use.

Hereinafter, the present invention will be described in more detail with reference to the following examples. These examples are provided to illustrate the present invention and should not be construed as limiting the scope and spirit of the present invention.

### EXAMPLES

Material and Methods

I. Differential Scanning calorimetry (DSC)
TA INSTRUMENTS Q200
Aluminium sealed sample pan (Not hermetic)
Atmosphere: Nitrogen
Heating rate: 10K/min
Data treatment: UNIVERSAL ANALYSIS 2000 v4.3

The samples were analyzed by DSC from 0° C. temperature up to 300° C.

II. X-Ray Powder Diffraction (XRPD)
Diffractometers Bruker D8 Advance;
Cupper anti cathode, tension 35 KV, intensity 40 mA
Bragg-Brentano configuration, fixed sample
Range of analysis: 2° to 40°
Step increment: 0.04°
Measuring time by step: is
Experimental treatment of the data by the EVA software (v 11.0)

The X-Ray peak positions and intensities are extracted from the analyzed samples.

III. HPLC-UV Method

Samples were analysed by HPLC-UV according to the following method:
Instrument: Agilent 1200 and 1100 with PDA detector
Column: Waters XTerra MS C18 3.5 μm 4.6×50 mm
Flow rate: 1.5 ml/min
UV detection: 220 nm, 270 nm, 290 nm
Column temperature: 40° C.
Injection volume: 1 μl
Mobile phase: gradient with solutions A and B prepared according to the following:
Eluent A: Ultrapurified water adjusted to pH2 with orthophosphoric acid 85%
Eluent B: Acetonitrile

The following table 7a shows the HPLC gradient method used for kinetic solubility measurements and dissolution/precipitation in vitro model.

TABLE 7a

| Time in minutes | % A | % B |
|---|---|---|
| 0 | 90 | 10 |
| 4 | 5 | 95 |
| 7.9 | 5 | 95 |
| 8 | 90 | 10 |
| 10 | 90 | 10 |

### Example 1: Preparation of a Co-Crystal of 8-Chloro-N-(4-(trifluoromethoxy)phenyl)quinolin-2-amine: L-Proline

6.6 mg of 8-chloro-N-(4-(trifluoromethoxy)phenyl)quinolin-2-amine were mixed with 1.6 mg of L-Proline as a co-former and 2 μl of acetonitrile and then the obtained mixture was grounded at 20 hz for 45 min using a Retsch MM200 instrument to obtain 7 mg of a co-crystal of 8-Chloro-N-(4-(trifluoromethoxy)phenyl)quinolin-2-amine: L-Proline.

The 8-chloro-N-(4-(trifluoromethoxy)phenyl)quinolin-2-amine: L-Proline co-crystal was characterized by X-Ray Powder Diffraction (XRPD) to confirm co-crystal formation and by Differential Scanning calorimetry (DSC) to determine the thermal behavior of new solid forms.

More particularly, this 8-chloro-N-(4-(trifluoromethoxy)phenyl)quinolin amine: L-Proline co-crystal has a powder X-ray diffractogram displaying peaks expressed as degree 2-Theta angle at 16.5; 20.6; 21.4; and 22.1 (each time ±0.2), and which may optionally further show the following additional peaks expressed as degree 2-Theta angle: 11.0; 15.9; 18.3; and 19.4 (each time ±0.2); and even optionally further the following additional peaks expressed as degree 2-Theta angle 6.1; 12.2; 12.6; 13.3; 13.7; 15.4; 17.3 and 22.4 (each time ±0.2), optionally further characterized by a powder X-ray diffractogram as illustrated in FIG. 1 and/or having a single endotherm with an onset temperature of 172.0° C. (±2° C.).

The same example was conducted except that acetonitrile was replaced by methanol as a polar organic solvent and co-crystal of 8-Chloro-N-(4-(trifluoromethoxy)phenyl)quinolin-2-amine: L-Proline co-crystal is obtained.

### Example 2: Preparation of a Co-Crystal of 8-Chloro-N-(4-(trifluoromethoxy)phenyl)quinolin-2-amine: Gentisic acid

8.9 mg of 8-chloro-N-(4-(trifluoromethoxy)phenyl)quinolin-2-amine were mixed with 2.3 mg of Gentisic acid as a co-former and 2 μl of acetonitrile and then the obtained mixture was grounded at 20 hz for 45 min using a Retsch MM200 instrument to obtain 7 mg of a co-crystal of 8-Chloro-N-(4-(trifluoromethoxy)phenyl)quinolin-2-amine: Gentisic acid.

The 8-chloro-N-(4-(trifluoromethoxy)phenyl)quinolin-2-amine: Gentisic acid co-crystal was characterized by X-Ray Powder Diffraction (XRPD) to confirm co-crystal formation and by Differential Scanning calorimetry (DSC) to determine the thermal behavior of new solid forms.

More particularly, this 8-chloro-N-(4-(trifluoromethoxy)phenyl)quinolin-2-amine: Gentisic acid co-crystal has a powder X-ray diffractogram displaying peaks expressed as degree 2-Theta angle at 7.9; 14.0; 15.2; and 25.2 (each time ±0.2), and which may optionally further show the following additional peaks expressed as degree 2-Theta angle: 15.8; 16.9; 18.5; 19.9; 20.3; 23.0 and 24.7 (each time ±0.2); and even optionally further the following additional peaks expressed as degree 2-Theta angle: 7.6; 14.7; 16.1; 19.7; 21.6; 22.0; 22.3; 23.7; and 24.0 (each time ±0.2), optionally further characterized by a powder X-ray diffractogram as illustrated in FIG. 2 and/or having a single endotherm with an onset temperature of 133.0° C. (±2° C.).

The same example was conducted except that acetonitrile was replaced by methanol as a polar organic solvent and co-crystal of 8-chloro-N-(4-(trifluoromethoxy)phenyl)quinolin-2-amine: Gentisic acid is obtained.

### Example 3: Preparation of a Co-Crystal of 8-Chloro-N-(4-(trifluoromethoxy)phenyl)quinolin-2-amine: Malonic acid

5.8 mg of 8-chloro-N-(4-(trifluoromethoxy)phenyl)quinolin-2-amine were mixed with 1.5 mg of Malonic acid as a co-former and 2 μl of acetonitrile and then the obtained mixture was grounded at 20 hz for 45 min using a Retsch MM200 instrument to obtain 7 mg of a co-crystal of 8-chloro-N-(4-(trifluoromethoxy)phenyl)quinolin-2-amine: Malonic acid

The 8-chloro-N-(4-(trifluoromethoxy)phenyl)quinolin-2-amine: Malonic acid co-crystal was characterized by X-Ray Powder Diffraction (XRPD) to confirm co-crystal formation and by Differential Scanning calorimetry (DSC) to determine the thermal behavior of new solid forms.

More particularly, this 8-chloro-N-(4-(trifluoromethoxy)phenyl)quinolin-2-amine: Malonic acid co-crystal has a powder X-ray diffractogram displaying peaks expressed as degree 2-Theta angle at 9.5; 12.2; 15.8; 17.3; 19.7; 22.8; 24.8; and 25.6 (each time ±0.2), and which may optionally further show the following additional peaks expressed as degree 2-Theta angle: 19.0; 21.4; 24.6; 26.8; 27.6; and 29.9 (each time ±0.2); and even optionally further the following additional peaks expressed as degree 2-Theta angle 16.8; 17.8; 20.9; 23.8; 28.0; and 29.6 (each time ±0.2), optionally further characterized by a powder X-ray diffractogram as illustrated in FIG. 3 and/or having a single endotherm with an onset temperature of 109.0° C. (±2° C.).

The same example was conducted except that acetonitrile was replaced by methanol as a polar organic solvent and co-crystal of 8-chloro-N-(4-(trifluoromethoxy)phenyl)quinolin-2-amine: Malonic acid is obtained.

### Example 4: Preparation of a Co-Crystal of 8-Chloro-N-(4-(trifluoromethoxy)phenyl)quinolin-2-amine: 4, 4′-Bipyridine

8.6 mg of 8-chloro-N-(4-(trifluoromethoxy)phenyl)quinolin-2-amine were mixed with 2.4 mg of 4, 4′-Bipyridine as a co-former and 2 μl of acetonitrile and then the obtained mixture was grounded at 20 hz for 45 min using a Retsch MM200 instrument to obtain 7 mg of a co-crystal of 8-Chloro-N-(4-(trifluoromethoxy)phenyl)quinolin-2-amine: of 4, 4′-Bipyridine.

The 8-chloro-N-(4-(trifluoromethoxy)phenyl)quinolin-2-amine: 4, 4′-Bipyridine co-crystal was characterized by X-Ray Powder Diffraction (XRPD) to confirm co-crystal formation and by Differential Scanning calorimetry (DSC) to determine the thermal behavior of new solid forms.

More particularly, this 8-chloro-N-(4-(trifluoromethoxy)phenyl)quinolin amine: 4, 4′-Bipyridine co-crystal has a powder X-ray diffractogram displaying peaks expressed as degree 2-Theta angle at 12.0; 19.2; 21.2; and 24.3 (each time ±0.2), and which may optionally further show the following additional peaks expressed as degree 2-Theta angle: 16.0; 17.0; 17.8; 20.3; 22.5; and 22.7 (each time ±0.2); and even optionally further the following additional peaks expressed as degree 2-Theta angle: 8.5; 13.0; 15.7; 16.7; 20.9; 22.0; 23.1; 23.6 and 24.7 (each time ±0.2), optionally further characterized by a powder X-ray diffractogram as illustrated in FIG. 4 and/or having a single endotherm with an onset temperature of 127.0° C. (±2° C.).

The same example was conducted except that acetonitrile was replaced by methanol as a polar organic solvent and co-crystal of 8-chloro-N-(4-(trifluoromethoxy)phenyl)quinolin-2-amine: 4, 4′-Bipyridine is obtained.

### Example 5: Preparation of Anhydrous Crystalline ABX464 Hemi-Napadisylate Salt

11.9 mg of 8-chloro-N-(4-(trifluoromethoxy)phenyl)quinolin-2-amine were dissolved in 500 μl of methanol. To this mixture were added 175 μl of naphthalene1,5disulfonic acid 0.1M (counter ion) in ethanol so as to obtain a ABX464: counter ion molar ratio of 2:1.

The mixture preparation was magnetic stirred at room temperature (25° C.) for ½ hour. Solvents were evaporated at room temperature (25° C.) under N₂ gas flow. Acetone was then added as crystallization solvent (1180 μl of acetone) into the tube. The tube was then hermetically closed to prevent evaporation of the crystallization solvent and heated 1 h at 60° C. while the solution was stirred by a magnetic bare. To induce crystallization of products, the tube was then cooled at a 0.1° C./min rate and to 5° C. while the solution was stirred by a magnetic bare. The obtained suspension was then filtered under 0.2 μm mesh and then drying under vacuum at 40° C.

8 mg of an anhydrous crystalline ABX464 hemi-napadisylate salt is thus obtained.

As already mentioned in the present text, this salt has a powder X-ray diffractogram displaying peaks expressed as degree 2-Theta angle at 9.8; 16.4; 18.2; 20.1; 21.2; 21.6; 23.5 and 26.3 (each time ±0.2), and optionally further shows the following additional peaks expressed as degree 2-Theta angle: 12.4; 13.1; 17.8; 20.9; 22.6; 24.5; 24.7; 25.2; and 25.9 (each time ±0.2); and even optionally further the following additional peaks expressed as degree 2-Theta angle: 8.8; 13.3; 15.1; 17.2; 17.5; 19.4; 19.5; and 19.8 (each time ±0.2), as illustrated in FIG. 5 (powder X-ray diffractogram) and/or having a single endotherm with an onset temperature of 269.0° C. (±2° C.).

A characteristic X-ray powder diffractogram of an anhydrous crystalline ABX464 hemi-napadisylate salt is thus given in FIG. 5.

### Example 6: Preparation of Anhydrous Crystalline ABX464 Esylate Salt

11.4 mg of 8-chloro-N-(4-(trifluoromethoxy)phenyl)quinolin-2-amine were dissolved in 500 μl of methanol. To this mixture were added 3370 of ethane sulfonic acid 0.1M (counter ion) in water/ethanol ( 1/10) so as to obtain a ABX464: counter ion molar ratio of 1:1.

The mixture preparation was magnetic stirred at room temperature (25° C.) for ½ hour. Solvents were evaporated at room temperature (25° C.) under N₂ gas flow. Ethyl Acetate was then added as crystallization solvent (480 μl of Ethyl Acetate) into the tube. The tube was then hermetically closed to prevent evaporation of the crystallization solvent and heated 1 h at 60° C. while the solution was stirred by a magnetic bare. To induce crystallization of products, the tube was then cooled at a 0.1° C./min rate and to 5° C. while the solution was stirred by a magnetic bare. The obtained solution was then evaporated under N₂ gas flow at room temperature.

12 mg of an anhydrous crystalline ABX464 esylate salt is thus obtained.

As already mentioned in the present text, this salt has a powder X-ray diffractogram displaying peaks expressed as degree 2-Theta angle at 12.2; and 22.2 (each time ±0.2), and optionally further shows the following additional peaks expressed as degree 2-Theta angle: 6.2; 12.9; 13.1; 15.3; 16.3; 18.2; 18.6; 19.5; 20.0; and 20.7 (each time ±0.2); and even optionally further the following additional peaks expressed as degree 2-Theta angle: 10.1; 15.8; 17.7; 17.9; 20.3; and 21.4 (each time ±0.2), as illustrated in FIG. 6 (powder X-ray diffractogram) and/or having a single endotherm with an onset temperature of 108.0° C. (±2° C.).

A characteristic X-ray powder diffractogram of anhydrous crystalline ABX464 esylate salt is thus given in FIG. 6.

### Example 7: Preparation of Crystalline Hemi-THF (Tetrahydrofuran) Solvate of ABX464 Hemi-Napadisylate Salt

10.6 mg of 8-chloro-N-(4-(trifluoromethoxy)phenyl)quinolin-2-amine were dissolved in 500 μl of methanol. To this mixture were added 313 μl of naphthalene1,5disulfonic acid 0.1M (counter ion) in ethanol so as to obtain a ABX464: counter ion molar ratio of 2:1.

The mixture preparation was magnetic stirred at room temperature (25° C.) for ½ hour. Solvents were evaporated at room temperature (25° C.) under N₂ gas flow. THF was then added as crystallization solvent (1030 μl of THF) into the tube. The tube was then hermetically closed to prevent evaporation of the crystallization solvent and heated 1 h at 60° C. while the solution was stirred by a magnetic bare. To induce crystallization of products, the tube was then cooled at a 0.1° C./min rate and to 5° C. while the solution was stirred by a magnetic bare. The obtained suspension was then filtered under 0.2 μm mesh and then drying under vacuum at 40° C.

8 mg of a crystalline hemi-THF (tetrahydrofuran) solvate of ABX464 hemi-napadisylate salt is thus obtained.

As already mentioned in the present text, this salt has a powder X-ray diffractogram displaying peaks expressed as degree 2-Theta angle at 8.4; 12.3; 14.0; 19.2; 21.3; 22.6 and 24.6 (each time ±0.2), and optionally further shows the following additional peaks expressed as degree 2-Theta angle: 9.6; 13.0; 13.5; 14.8; 17.2; 17.8; 23.4; 24.1; 24.9 and 25.2 (each time ±0.2); and even optionally further the following additional peaks expressed as degree 2-Theta angle: 16.7; 18.1; 18.8; 19.5; 20.9 and 22.3 (each time ±0.2), as illustrated in FIG. 7 (powder X-ray diffractogram) and/or having a single endotherm with an onset temperature of 172.0° C. (±2° C.).

A characteristic X-ray powder diffractogram of a crystalline hemi-THF solvate of ABX464 hemi-napadisylate salt is thus given in FIG. 7.

### Example 8: Pharmaceutical Compositions Under the Form of a Capsule in Accordance with the Invention Comprising a Co-Crystal of ABX464 as Defined in the Invention

The following capsule was prepared with the ingredients in the respective amounts as specified below in the table 8.

TABLE 8

| | | Amount |
|---|---|---|
| Ingredients | Function | (in mg)/unit |
| 8-chloro-N-(4-(trifluoro- | Active ingredient | 20.00 equivalents |
| methoxy)phenyl)quinolin-2- | | ABX464 |
| amine:4,4′-Bipyridine | | |
| co-crystal as prepared | | |
| according to example 4 | | |
| MANNITOL | Filler | 120.00 |
| PREGELATINIZED | Binder | 10.00 |
| STARCH | | |
| TALC | Glidant | 0.85 |
| ZINC STEARATE | Lubricant | 0.85 |
| White opaque hard gelatin | Capsule shell | 1 unit |
| capsule, size 1 sold | | |
| by Capsugel Belgium NV | | |
| (Body composition: 2% TiO₂ | | |
| and qsp 100% gelatin | | |
| (bovine and/or porcine origin) | | |
| Cap composition: 2% TiO₂ | | |
| and qsp 100% gelatin | | |
| (bovine and/or porcine origin)). | | |

The pharmaceutical compositions in accordance with the invention are useful in the prevention and/or treatment of inflammatory diseases such as Inflammatory Bowel Disease, Rheumatoid Arthritis, pulmonary arterial hypertension, NASH (nonalcoholic steatohepatitis) and Multiple Sclerosis, diseases caused by viruses and/or cancer or dysplasia.

### Example 9: Pharmaceutical Composition Under the Form of a Capsule in Accordance with the Invention Comprising a Pharmaceutically Acceptable Salt of ABX464 as Defined in the Invention

The following capsule was prepared with the ingredients in the respective amounts as specified below in the table 9.

TABLE 9

| | | Amount |
|---|---|---|
| Ingredients | Function | (in mg)/unit |
| Crystalline hemi-THF | Active ingredient | 20.00 equivalents |
| solvate of ABX464 hemi- | | ABX464 |
| napadisylate salt as prepared | | |
| according to example 7 | | |
| MANNITOL | Filler | 120.00 |
| PREGELATINIZED | Binder | 10.00 |
| STARCH | | |
| TALC | Glidant | 0.85 |
| ZINC STEARATE | Lubricant | 0.85 |
| White opaque hard gelatin | Capsule shell | 1 unit |
| capsule, size 1 sold by | | |
| Capsugel Belgium NV | | |
| (Body composition: 2% TiO₂ | | |
| and qsp 100% gelatin | | |
| (bovine and/or porcine origin) | | |
| Cap composition: 2% TiO₂ | | |
| and qsp 100% gelatin | | |
| (bovine and/or porcine origin)). | | |

The pharmaceutical compositions in accordance with the invention are useful in the treatment and/or prevention of inflammatory diseases such as Inflammatory Bowel Disease, Rheumatoid Arthritis, pulmonary arterial hypertension, NASH and Multiple Sclerosis, diseases caused by viruses and/or cancer or dysplasia.

### Example 10: Preparations and Solubility Kinetic Measurement of Anhydrous ABX464 Hemi-Napadisylate Salt and ABX464: L-Proline Co-Crystal in Accordance with the Disclosure Compared to ABX464 Crystalline Form I, in Two Different Specific Aqueous Media at Selected Sampling Time

The anhydrous ABX464 Hemi-Napadisylate salt and the ABX464: L-Proline co-crystal in accordance with the disclosure which were tested, were prepared according to the two protocols detailed below (see respectively example 10a and example 10b).

### Example 10a: Preparation of Anhydrous ABX464 Hemi-Napadisylate Salt

The synthesis of anhydrous ABX464 Hemi-Napadisylate salt was performed according to the following protocol:
200 mg of ABX464 Crystalline Form I were weighed in a 20 mL glass vial;
10 ml of Methanol was added in the glass vial to solubilize ABX464 Crystalline Form I using 15 min magnetically stirring at room temperature;
ABX464 Methanol solution was then filtered through PTFE 0.45 μm filter.
5.9 mL of Naphthalene 1,5 Disulfonic acid 0.05M Ethanol solution was added drop by drop at room temperature;
Solution was magnetically stirred for 60 minutes at room temperature (a clear yellow solution was noted);
Solvents were then evaporated under N₂ gaz flow at room temperature (a slightly yellow solid was obtained);
10 mL of Acetone were added in the glass vial at room temperature to crystallize ABX464 hemi Napadisylate salt overnight while suspension was magnetically stirred;
The suspension was filtered through 0.2 μm cellulose mesh filter (a slightly yellow solid was obtained);
The obtained solid was finally dried under vacuum at room temperature to get the anhydrous ABX464 Hemi-Napadisylate salt.

In order to characterize this salt, a X-Ray Powder Diffraction analysis was performed. The corresponding XRPD pattern was characteristic of a crystalline solid and was comparable to the one illustrated in FIG. 5.

### Example 10b: Preparation of ABX464: L-Proline Co-Crystal

The synthesis of ABX464: L-Proline Co-crystal was performed according to the following protocol:
The powder physical mixture was prepared by mixing 200 mg of ABX464 Crystalline Form I and 68 mg of L-Proline (1/1 molar stoichiometry) in a 10 ml stainless steel milling jar
2 stainless steel milling balls of 5 mm diameter were introduced in the milling jar;
130 μl of Methanol were added;
Immediately after addition of solvent drops, the jar was hermetically closed;
The jar was then placed on the milling instrument (Retsch MM200) for 45 min milling at 20 Hz;
The ABX464: L-Proline co-crystal was thus obtained.

In order to characterize this co-crystal, a X-Ray Powder Diffraction analysis was performed. The corresponding XRPD pattern was characteristic of a crystalline solid and was comparable to the one illustrated in FIG. 1.

### Example 10c: Kinetic Solubility in Two Aqueous Media

Kinetic solubility measurements for ABX464 crystalline Form I, for anhydrous ABX464 hemi-Napadisylate salt and for ABX464: L-Proline co-crystal respectively have been carried out using the following protocol.

Kinetic Solubility Protocol
Sample preparation for kinetic solubility measurements at room temperature protected from light at 2 mg/ml (equivalent ABX464) target concentration:
   In a 4 ml glass vial, 2 mg equivalent ABX464 of tested material were weighed;
   At room temperature 1 ml of the selected medium (see below for details) was added;
   At room temperature protected from light orbital stirring was made (see below);
   At selected sampling time (5 min, 30 min, 2 h, 4 h and 24 h), the soluble fraction from the non-soluble was separated by filtration on PTFE 0.45 μm membrane;
   Immediately after filtration, HPLC-UV dosage of resulting filtrate versus external single point calibration was carried out. The HPLC-UV method selected for these measurements is described above (see also table 7a above).
Preparation of ABX464 reference solution at 1 mg/ml:
   1 mg of ABX464 Crystalline Form I was weighed;
   it was solubilized with DMSO until a 1 mg/ml clear DMSO solution was obtained.

These two specific aqueous media are more particularly:
FaSSIF (namely Fasted State Simulated Intestinal Fluid) pH=6.5+1% by weight of PVPVA (polyvinyl pyrrolidone-vinyl acetate: a precipitation inhibitor); and
FeSSIF (namely Fed State Simulated Intestinal Fluid) pH=5.0+1% by weight of PVPVA (a precipitation inhibitor)

To make 5 mL of FaSSIF, the steps were as follows:
Weighing 11.2 mg of SIF Powder, Adding 175 μl of FaSSIF Buffer Concentrate then making up to 5.0 mL with ultrapurified water; Letting stand for 2 hours; and adding 1% w/w PVPVA.

To make 5 mL of FeSSIF the steps were as follows:
weighing 56.0 mg of SIF Powder, adding 350 μl of FeSSIF Buffer Concentrate then making up to 5.0 mL with ultrapurified water; and adding 1% w/w PVPVA.

Kinetic Solubility Results

The kinetic solubility results of ABX464 crystalline Form I, of anhydrous ABX464 hemi-Napadisylate salt and of ABX464: L-Proline co-crystal in the FassiF and FessiF media at different sample times are reported in Table 10 below.

TABLE 10

| | | Kinetic solubility (μg/ml) | | |
|---|---|---|---|---|
| | | | Anhydrous | |
| | | ABX 464 | ABX464 hemi- | ABX464:L- |
| | Sampling | crystalline | Napadisylate | Proline |
| Media | times | Form 1 | salt | co-crystal |
| FassiF | 5 min | 6 | 70 | 80 |
| pH 6.5 + 1% | 30 min | 22 | 116 | 114 |
| PVPVA | 2 h | 47 | 149 | 118 |
| | 4 h | 62 | 206 | 107 |
| | 24 h | 72 | 432 | 98 |
| FessiF | 5 min | 54 | 396 | 869 |
| pH 5 + 1% | 30 min | 153 | 782 | 1064 |
| PVPVA | 2 h | 297 | 1053 | 1162 |
| | 4 h | 358 | 1055 | 1277 |
| | 24 h | 419 | 1517 | 1279 |

As conclusion of these solubility measurements, the following general observations can be highlighted:
In both media and at each time, there is a higher solubility of anhydrous ABX464 hemi-Napadisylate salt and of ABX464: L-Proline co-crystal compared to the ABX464 crystalline Form I.
There is a significant higher solubility of all solid forms in FeSSIF pH5.0+1% PVPVA medium.

Finally, it was noted that the solubility of:
anhydrous ABX464 hemi-Napadisylate salt in FeSSIF+1% PVPVA medium reaches rapidly a plateau at ˜1.05 mg/ml at 2 h before to continue to increase up to ˜1.52 mg/ml at 24 h.
ABX464: L-Proline co-crystal in FeSSIF+1% PVPVA medium reaches rapidly a plateau at ˜1.16 mg/ml at 2 h before to weakly continue to increase up to ˜1.28 mg/ml at 24 h.
ABX464 Crystalline Form I in FeSSIF+1% PVPVA medium reaches a plateau at ˜0.30 mg/ml at 2 h before to weakly continue to increase up to ˜0.42 mg/mL at 24 h.

### Example 11: Solubility Measurement in the Intestinal Compartment of the Two Step Dissolution-Precipitation Fasted Human In Vitro Model of Anhydrous ABX464 Hemi-Napadisylate Salt Compared to ABX464 Crystalline Form I

The ABX464 crystalline Form I, and anhydrous ABX464 hemi-Napadisylate salt have been evaluated in the Two-steps Dissolution/Precipitation in vitro Human model.

The anhydrous ABX464 Hemi-Napadisylate salt used in this test was the one prepared according to the protocol detailed in example 10a above.

The ABX464 crystalline Form I used in this test was the same as the one used in example 10 above.

The model consists in the following steps:
Dispensing selected material in Fasted simulated gastric medium containing 1% w PVPVA (inhibitor of precipitation) and then after 30 min,
Diluting obtained suspension by simulated Fasted intestinal medium.

Using this model, the dissolution of selected materials (ABX464 crystalline Form I, or anhydrous BX464 hemi-Napadisylate salt) in gastric medium and the risk of precipitation of the API (Active Pharmaceutical Ingredient) in intestinal compartment is evaluated.

The crystalline nature of solid residues collected by end of each steps (Gastric and Intestinal) have been determined by XRPD after centrifugation (15 min at 18000 rpm).

Protocol for Dissolution/Precipitation Model in Human Fasted Conditions
In 6 different vials, 4.0 mg equivalent ABX464 of selected material are weighed
Solubility measurement in the gastric compartment (also named G) after 15 min (G15 min) and after 30 min (G30 min) which comprises the following steps:
   Adding 1 mL of FaSSGF pH 1.2 medium containing 1% w PVPVA (ABX464 target concentration=4 mg/ml) on the 6 vials,
   Mixing with a vortex at 37° C.
   At times 15 min and 30 min, considering one vial (vials labelled G15 min and G30 min respectively) and for each vials
      noting the aspect of suspension,
      centrifuging 5 min at 18000 rpm and filtering on a 0.45 μm filter (filter Millex LCR Ref. SLCR0,13NK).
      Measuring the pH of supernatant,
      Dosing ABX464 soluble fraction by HPLC,
      Recording XPRD pattern on the final time point sample.
Solubility measurement in the intestinal compartment (also named I) after 15 min (vial I 15 min), 30 min (vial I 30 min), 60 min (vial I 60 min) and 120 min (vial I 120 min) which comprises the following steps:
   Adding 1 mL of FaSSIF ×2 pH 6.5 medium/sodium bicarbonate 90/10) on the 4 remaining vials (ABX464 target concentration=2 mg/ml)
   Mixing with a Vortex at 37° C.
   At times 15 min, 30 min, 60 min and 120 min, considering one vial (vials labelled I 15 min, I 30 min, I 60 min and I 120 min respectively) and for each vials
      noting the aspect of suspension
      Centrifuging 5 min at 18000 rpm and filtering on a 0.45 μm filter (filter Millex LCR Ref. SLCR0,13NK).
      Measuring the pH of supernatant
      Dosing ABX464 soluble fraction by HPLC
      Recording XPRD pattern on the final time point sample.

Composition and preparation of biorelevant media in Fasted conditions (see tables 11 and 12):
Fasted Gastric medium=FaSSGF pH=1.2/1% PVPVA w/v

TABLE 11

| Product | Concentration | Preparation (500 mL) |
|---|---|---|
| NaCl | 34 nM | 1.0 g of sodium chloride (NaCl) was |
| HCl | qs pH 1.2 | weighed in about 400 mL of deionized |
| PVPVA | 1% w | water. 5 g of PVPVA were added. NaCl and |
| water | qs 500 mL | PVPVA were dissolved by magnetically |
| | | stirring. The pH was adjusted to 1.2 with |
| | | HCl. Then it was made up to 500 mL with |
| | | deionized water. |

Fasted Intestinal medium (FaSSIF X2 pH=6.5/Sodium Bicarbonate at 80 g/l 90/10 v/v) Composition/Preparation of FaSSIF ×2 (medium concentrated 2 times to take into account the dilution in the Fasted model):

TABLE 12

| Product | Concentration | Preparation (500 mL) |
|---|---|---|
| Na taurocholate | 6 mM | 1) Preparation buffer: in |
| (TC) | | about 0.45 L of deionized |
| Lecithin S100 (L) | 1.5 mM | water |
| NaH₂PO₄, 2H₂O | 65.9 mM | 2 × 0.21 = 0.42 g of Sodium |
| NaCl | 212 mM | hydroxide pellets (NaOH) |
| NaOH 1N or | qs pH 6.5 | 2 × 1.98 = 3.95 g of NaH₂PO₄, |
| HCl 1N | | 2H₂O |
| water | qs 500 mL | 2 × 3.1 = 6.19 g of Sodium |
| | | chloride (NaCl) |
| | | the pH was adjusted to 6.5 |
| | | with NaOH 1N or HCl 1N. |
| | | it was made up to volume |
| | | (0.5 L) with purified water at |
| | | room temperature. |
| | | 2) SIF powder (from |
| | | Biorelevant) was added |
| | | 2 × 1.12 = 2.24 g of SIF |
| | | Powder were weighed. |
| | | 0.25 L of buffer at room |
| | | temperature was added. |
| | | Powder was stirred until it was |
| | | completely dissolved. |
| | | It was made up to 0.5 L. |
| | | Remarks: Let stand for 2 hours, |
| | | stability 48 h at room |
| | | temperature |

The FaSSIF X2/Sodium Bicarbonate 80 g/l (90/10; v/v) mixture was prepared to keep pH equal to 6.5 in the intestinal compartment after dilution.

Results of Two-Steps Dissolution/Precipitation Human Fasted Model

Solubility values were obtained for ABX464 Crystalline Form I and anhydrous ABX464 hemi-Napadisylate salt in the Two-Steps Dissolution/Precipitation human Fasted model as reported in Table 13 to Table 16 below.

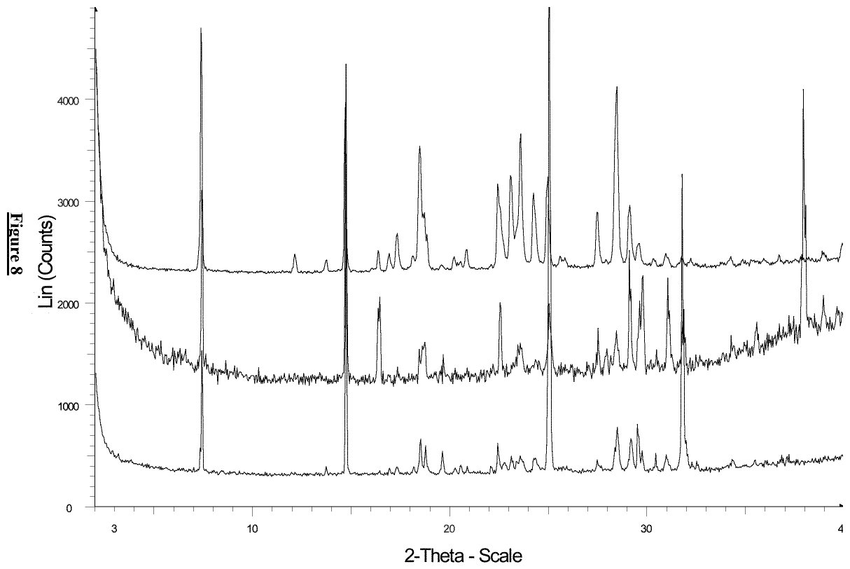

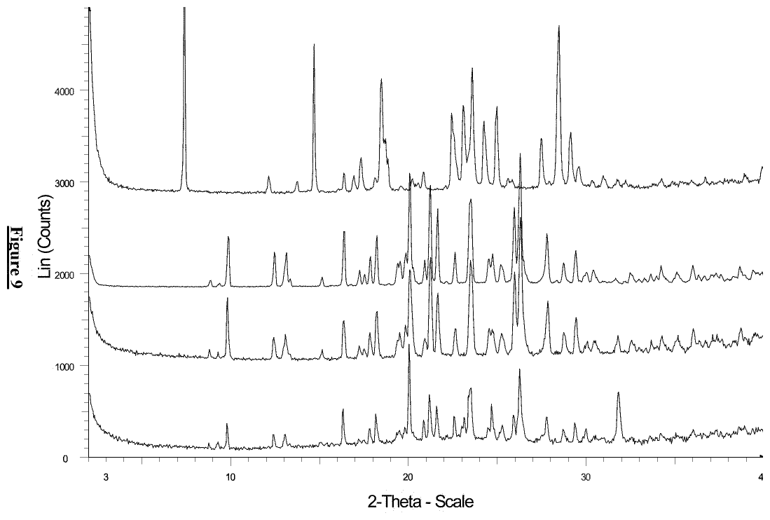

XRPD patterns recorded for solid residues collected at last time points in Gastric (G 30 min) and Intestinal (I 120 min) compartments of Fasted Dissolution/Precipitation tests were done for ABX464 crystalline Form I, and anhydrous ABX464 hemi-Napadisylate salt and were reported on FIG. 8 (bottom line) and FIG. 9 (bottom line) respectively.

In FIG. 8, a X-ray powder diagram illustrating ABX464 crystalline Form I (top line) was also included for comparison.

In FIG. 9, two X-ray powder diagrams illustrating ABX464 crystalline Form I (top line) and anhydrous ABX464 hemi-Napadisylate salt (second line from the top) were also included for comparison.

The data and results (solubility measured in fasted model with 1% PVPVA in FaSSGF pH1.2) for ABX464 crystalline Form I are gathered in the below Table 13 and Table 14.

TABLE 13

| | | Mass of | Mass of AP in |
|---|---|---|---|
| | Time | Active Principle | active form |
| Vials | (min) | (AP) (mg) | (mg) |
| G 15 min | 15 | 4.09 | 4.09 |
| G 30 min | 30 | 4.07 | 4.07 |
| I 15min | 45 | 4.16 | 4.16 |
| I 30 min | 60 | 4.09 | 4.09 |
| I 60 min | 90 | 4.02 | 4.02 |
| I 120 min | 150 | 4.23 | 4.23 |

TABLE 14

| | Gastric | | | | |
|---|---|---|---|---|---|
| | or | | | Target | |
| | Intestinal | | pH | concen- | Soluble |
| | volume | Observation | (at a given | tration | fraction |
| Vials | (mL) | (at a given time) | time) | (mg/mL) | (mg/mL) |
| G | 1 | Clear and | 1.2 | 4.09 | 0.06 |
| 15 min | | colorless, white | | | |
| | | particles in | | | |
| | | suspension | | | |
| G | 1 | Clear and | 1.2 | 4.07 | 0.07 |
| 30 min | | colorless, white | | | |
| | | particles in | | | |
| | | suspension | | | |
| I | 1 | Clear and | 6.3 | 2.08 | 0.06 |
| 15 min | | colorless, white | | | |
| | | particles in | | | |
| | | suspension | | | |
| I | 1 | Clear and | 6.3 | 2.05 | 0.07 |
| 30 min | | colorless, white | | | |
| | | particles in | | | |
| | | suspension | | | |
| I | 1 | Clear and | 6.3 | 2.01 | 0.07 |
| 60 min | | colorless, white | | | |
| | | particles in | | | |
| | | suspension | | | |
| I | 1 | Clear and | 6.3 | 2.12 | 0.08 |
| 120 min | | colorless, white | | | |
| | | particles in | | | |
| | | suspension | | | |

The data and results (solubility measured in fasted model with 1% PVPVA in FaSSGF pH1.2) for anhydrous ABX464 hemi-Napadisylate salt are gathered in the below Table 15 and Table 16.

TABLE 15

| | Time | Mass of Active | Mass of AP in |
|---|---|---|---|
| Vials | (min) | Principle (AP) (mg) | active form (mg) |
| G 15 min | 15 | 5.88 | 4.11 |
| G 30 min | 30 | 5.78 | 4.04 |
| I 15 min | 45 | 5.88 | 4.11 |
| I 30 min | 60 | 5.75 | 4.02 |
| I 60 min | 90 | 5.78 | 4.04 |
| I 120 min | 150 | 5.88 | 4.11 |

TABLE 16

| | Gastric | | | | |
|---|---|---|---|---|---|
| | or | | | Target | |
| | Intestinal | | pH | concen- | Soluble |
| | volume | Observation | (at a given | tration | fraction |
| Vials | (mL) | (at a given time) | time) | (mg/mL) | (mg/mL) |
| G | 1 | Opalescent | 1.2 | 4.11 | 0.05 |
| 15 min | | beige | | | |
| | | solution with | | | |
| | | particles in | | | |
| | | suspension | | | |
| G | 1 | Opalescent | 1.2 | 4.04 | 0.06 |
| 30 min | | beige | | | |
| | | solution with | | | |
| | | particles in | | | |
| | | suspension | | | |
| I | 1 | Opalescent | 6.2 | 2.06 | 0.17 |
| 15 min | | beige | | | |
| | | solution with | | | |
| | | particles in | | | |
| | | suspension | | | |
| I | 1 | Opalescent | 6.2 | 2.01 | 0.23 |
| 30 min | | beige | | | |
| | | solution with | | | |
| | | particles in | | | |
| | | suspension | | | |
| I | 1 | Opalescent | 6.2 | 2.02 | 0.32 |
| 60 min | | beige | | | |
| | | solution with | | | |
| | | particles in | | | |
| | | suspension | | | |
| I | 1 | Opalescent | 6.2 | 2.06 | 0.43 |
| 120 min | | beige | | | |
| | | solution with | | | |
| | | particles in | | | |
| | | suspension | | | |

From these results, the following points can be highlighted:
In the Intestinal compartment at pH 6.5:
   No significant evolution of the ABX464 soluble fraction starting from the ABX464 crystalline Form I in Intestinal compartment (80 μg/ml) and no crystalline phase conversion as detected by XRPD.
   Starting from anhydrous ABX464 hemi-Napadisylate salt, a significant increase of ABX464 soluble fraction in the Intestinal compartment (430 μg/ml) is noted and no crystalline phase conversion as detected by XRPD.

The main conclusions of these solubility studies as shown on examples 10 and 11 above were:
A significant higher solubility for ABX464 hemi-Napadisylate salt and for ABX464: L-Proline co-crystal, in Fassif and Fessif media, compared to the ABX464 crystalline Form I.
A significant higher solubility of ABX464 hemi-Napadisylate salt (430 μg/ml) in the intestinal compartment of the Two Steps Dissolution/Precipitation Fasted in-vitro human model compared to the solubility of ABX464 Crystalline Form I (80 μg/ml) in the intestinal compartment of this model.

1. Co-crystals of 8-chloro-N-(4-(trifluoromethoxy)phenyl)quinolin-2-amine which are chosen among:
8-Chloro-N-(4-(trifluoromethoxy)phenyl)quinolin-2-amine: L-Proline having a powder X-ray diffractogram displaying peaks expressed as degree 2-Theta angle at 16.5; 20.6; 21.4; and 22.1 (each time ±0.2), and which may optionally further show the following additional peaks expressed as degree 2-Theta angle: 11.0; 15.9; 18.3; and 19.4 (each time ±0.2); and even optionally further the following additional peaks expressed as degree 2-Theta angle 6.1; 12.2; 12.6; 13.3; 13.7; 15.4; 17.3 and 22.4 (each time ±0.2) and/or having a single endotherm with an onset temperature of 172.0° C. (±2° C.);
8-Chloro-N-(4-(trifluoromethoxy)phenyl)quinolin-2-amine: Gentisic acid having a powder X-ray diffractogram displaying peaks expressed as degree 2-Theta angle at 7.9; 14.0; 15.2; and 25.2 (each time ±0.2), and which may optionally further show the following additional peaks expressed as degree 2-Theta angle: 15.8; 16.9; 18.5; 19.9; 20.3; 23.0 and 24.7 (each time ±0.2); and even optionally further the following additional peaks expressed as degree 2-Theta angle: 7.6; 14.7; 16.1; 19.7; 21.6; 22.0; 22.3; 23.7; and 24.0 (each time ±0.2), and/or having a single endotherm with an onset temperature of 133.0° C. (±2° C.);
8-Chloro-N-(4-(trifluoromethoxy)phenyl)quinolin-2-amine: Malonic acid having a powder X-ray diffractogram displaying peaks expressed as degree 2-Theta angle at 9.5; 12.2; 15.8; 17.3; 19.7; 22.8; 24.8; and 25.6 (each time ±0.2), and which may optionally further show the following additional peaks expressed as degree 2-Theta angle: 19.0; 21.4; 24.6; 26.8; 27.6; and 29.9 (each time ±0.2); and even optionally further the following additional peaks expressed as degree 2-Theta angle 16.8; 17.8; 20.9; 23.8; 28.0; and 29.6 (each time ±0.2), and/or having a single endotherm with an onset temperature of 109.0° C. (±2° C.); and
8-Chloro-N-(4-(trifluoromethoxy)phenyl)quinolin-2-amine: 4, 4′-Bipyridine having a powder X-ray diffractogram displaying peaks expressed as degree 2-Theta angle at 12.0; 19.2; 21.2; and 24.3 (each time ±0.2), and which may optionally further show the following additional peaks expressed as degree 2-Theta angle: 16.0; 17.0; 17.8; 20.3; 22.5; and 22.7 (each time ±0.2); and even optionally further the following additional peaks expressed as degree 2-Theta angle: 8.5; 13.0; 15.7; 16.7; 20.9; 22.0; 23.1; 23.6 and 24.7 (each time ±0.2), and/or having a single endotherm with an onset temperature of 127.0° C. (±2° C.).

2. A method for preparing a co-crystal of 8-chloro-N-(4-(trifluoromethoxy)phenyl)quinolin-2-amine as defined in claim 1 which comprises the following steps:
a) dissolving ABX464 in a solvent or in a mixture of solvents;
b) adding to the thus obtained mixture of step a) a co-former which may be itself already dissolved in a solvent or in a mixture of solvents and which is selected from L-proline, malonic acid, gentisic acid and 4,4′-bipyridine so as to obtain a ABX464: co-former in molar ratio comprised between 3:1 and 1:2;
c) optionally evaporating the solvent(s) at a temperature comprised between 0° C. and the boiling point of the selected solvent(s) or mixture of solvent(s) of step a) and step b);
d) optionally adding a solvent or a mixture of solvents,
e) applying a temperature program;
f) optionally filtrating; and
g) then optionally drying at a temperature comprised between room temperature and 60° C. in order to obtain the desired co-crystal of ABX464;
or which comprises the following steps:
a′) physically mixing 8-chloro-N-(4-(trifluoromethoxy)phenyl)quinolin-2-amine with a co-former chosen among L-Proline, Gentisic acid, Malonic acid and 4, 4′-Bipyridine at a molar ratio comprised between 2:1 and 1:2, molar ratio, in a suitable solvent or mixtures of solvents; and
b′) grinding of the physical mixture thus obtained from step a), in presence of one drop of solvent or of mixtures of solvents to obtain the co-crystal.

3. The method according to claim 2, wherein the solvent(s) of step a), step b) and/or step d) or of step a′) and/or step b′) is(are) any solvent conventionally used in the crystallization step, optionally in admixture with water, and mixtures thereof.

4. A pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

5. The pharmaceutically acceptable salt of 8-chloro-N-(4-(trifluoromethoxy)phenyl)quinolin-2-amine including a solvate and/or a hydrate thereof according to claim 4 which is chosen among:
anhydrous crystalline ABX464 hemi-napadisylate salt having a powder X-ray diffractogram displaying peaks expressed as degree 2-Theta angle at 9.8; 16.4; 18.2; 20.1; 21.2; 21.6; 23.5 and 26.3 (each time ±0.2), and optionally further shows the following additional peaks expressed as degree 2-Theta angle: 12.4; 13.1; 17.8; 20.9; 22.6; 24.5; 24.7; 25.2; and 25.9 (each time ±0.2); and even optionally further the following additional peaks expressed as degree 2-Theta angle: 8.8; 13.3; 15.1; 17.2; 17.5; 19.4; 19.5; and 19.8 (each time ±0.2), and/or having a single endotherm with an onset temperature of 269.0° C. (±2° C.);
anhydrous crystalline ABX464 esylate salt having a powder X-ray diffractogram displaying peaks expressed as degree 2-Theta angle at 12.2; and 22.2 (each time ±0.2), and optionally further shows the following additional peaks expressed as degree 2-Theta angle: 6.2; 12.9; 13.1; 15.3; 16.3; 18.2; 18.6; 19.5; 20.0; and 20.7 (each time ±0.2); and even optionally further the following additional peaks expressed as degree 2-Theta angle: 10.1; 15.8; 17.7; 17.9; 20.3; and 21.4 (each time ±0.2), and/or having a single endotherm with an onset temperature of 108.0° C. (±2° C.); and
crystalline hemi-THF solvate of ABX464 hemi-napadisylate salt having a powder X-ray diffractogram displaying peaks expressed as degree 2-Theta angle at 8.4; 12.3; 14.0; 19.2; 21.3; 22.6 and 24.6 (each time ±0.2), and optionally further shows the following additional peaks expressed as degree 2-Theta angle: 9.6; 13.0; 13.5; 14.8; 17.2; 17.8; 23.4; 24.1; 24.9 and 25.2 (each time ±0.2); and even optionally further the following additional peaks expressed as degree 2-Theta angle: 16.7; 18.1; 18.8; 19.5; 20.9 and 22.3 (each time ±0.2), and/or having a single endotherm with an onset temperature of 172.0° C. (±2° C.).

6. The method for preparing a pharmaceutically acceptable salt of ABX464 including solvate or hydrate thereof as defined in claim 4 which comprises the following steps:
a) dissolving ABX464 in a solvent or in a mixture of solvents;
b) adding to the thus obtained mixture of step a) a counter ion under the form of an acid which may be itself already dissolved in a solvent or in a mixture of solvents so as to obtain a ABX464: counter ion molar ratio comprised between 3:1 and 1:2;
c) optionally evaporating the solvent(s) at a temperature comprised between 0° C. and the boiling point of the selected solvent(s) or mixture of solvent(s) of step a) and step b);
d) optionally adding a solvent or a mixture of solvents,
e) applying a temperature program;
f) optionally filtrating; and
g) then optionally drying at a temperature comprised between room temperature and 60° C. in order to obtain the desired salt of ABX464.

7. The method according to claim 6, wherein the solvent of step a), step b) and/or step d) is any solvent conventionally used in the crystallization step; and/or
the counter ion under the form of an acid of step b) is ethane sulfonic acid or naphthalene1,5-disulfonic acid.

8. The method according to claim 2, wherein the step e) relative to the temperature program includes i) a heating at reflux to a temperature comprised between room temperature and the boiling point of the solvent(s), and/or ii) a cooling at reflux to a temperature comprised between 0° C. and 60° C., at a rate comprised between 30° C./min and 0.05° C./min.

9. The method according to claim 2, wherein the step f) regarding the filtration is carried out using conventional glass fibers, conventional cellulosic filter papers, PTFE (polytetrafluoroethylene), or PVDF (polyvinylidene fluoride).

10. The method according to claim 2, wherein the step g) regarding the drying is carried out under vacuum at a temperature comprised between 30° C. and 60° C., or under ambient atmosphere at a temperature comprised between 30° C. and 60° C.

11. Pharmaceutical composition comprising:
at least one of the co-crystals as defined in claim 1 and/or at least one pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid, and
at least one pharmaceutically acceptable excipient.

12. A medicament comprising:
the co-crystals of 8-chloro-N-(4-(trifluoromethoxy)phenyl)quinolin-2-amine as defined in claim 1, or a pharmaceutically acceptable salt of 8-chloro-N-(4-(trifluoromethoxy)phenyl)quinolin-2-amine as defined in claim 4 including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

13. A method for preventing and/or treating an inflammatory disease, comprising administering to a patient in need thereof the co-crystals of 8-chloro-N-(4-(trifluoromethoxy)phenyl)quinolin-2-amine as defined in claim 1, or a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

14. A method for preventing and/or treating cancer, comprising administering to a patient in need thereof the co-crystals of 8-chloro-N-(4-(trifluoromethoxy)phenyl)quinolin-2-amine as defined in claim 1, or a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

15. The method according to claim 12, wherein a presence and/or expression level of miR-124 in a blood and/or tissue sample of the patient, is measured prior to and/or during the administration.

16. A method for preventing and/or treating diseases caused by viruses, comprising administering to a patient in need thereof the co-crystals of 8-chloro-N-(4-(trifluoromethoxy)phenyl)quinolin-2-amine as defined in claim 1, or a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

17. A method for preventing and/or treating diseases caused by a virus belonging to Coronaviridae family or by a Coronaviridae infection and conditions related thereto, comprising administering to a patient in need thereof the co-crystals of 8-chloro-N-(4-(trifluoromethoxy)phenyl)quinolin-2-amine as defined in claim 1, or a pharmaceutically acceptable salt of 8-chloro-N-(4-(trifluoromethoxy)phenyl)quinolin-2-amine including a solvate and/or a hydrate thereof which is selected from lactate, oleate, oxalate, palmitate, stearate, valerate, pantothenate, picrate, butyrate, malonate, succinate, bitartrate, malate, mandelate, benzoate, edetate, gluceptate, gluconate, lactobionate, salicylate, disalicylate, mucate, pamoate, adipate, alginate, aspartate, camphorate, cyclopentaneproprionate, digluconate, glucoheptonate, heptanoate, hexanoate, laurate, nicotinate, pamoate, pivalate, propionate, undecanoate, phosphate, camphorsulfonate, 2-hydroxy-ethanesulfonate, estolate, napsylate, esylate, napadisylate, dodecylsulfate, perchloric acid, boric acid, glycerophosphoric acid, nitric acid, and persulfuric acid.

18. The method according to claim 12, wherein the level of 8-chloro-N-(4-(trifluoromethoxy)phenyl)quinolin-2-amine free base equivalent, in a blood, plasma, tissue, saliva, and/or serum sample of the patient is measured during the administration.
